SEQUENCE LISTING (358 sequences)

SEQ ID NO: 1 - protein, 9 aa, Artificial Sequence
  Wilm′s tumor 1 (WT1) peptide
RMFPNAPYL

SEQ ID NO: 2 - protein, 10 aa, Artificial Sequence
  MART-1 peptide
ELAGIGILTV

SEQ ID NO: 3 - protein, 261 aa, Artificial Sequence
  HLA*0201 extracellular domain
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHV

SEQ ID NO: 4 - protein, 8 aa, Artificial Sequence
  HPV E7 peptide
YMLDLQPE

SEQ ID NO: 5 - protein, 14 aa, Artificial Sequence
  HPV E7 peptide
YMLDLQPETTDLYC

SEQ ID NO: 6 - protein, 9 aa, Artificial Sequence
  HPV E7 peptide
YMLDLQPET

SEQ ID NO: 7 - protein, 12 aa, Artificial Sequence
  HPV E7 peptide
YMLDLQPETTDL

SEQ ID NO: 8 - protein, 9 aa, Artificial Sequence
  HPV E7 peptide
LLMGTLGIV

SEQ ID NO: 9 - protein, 8 aa, Artificial Sequence
  HPV E7 peptide
TLGIVCPI

SEQ ID NO: 10 - protein, 9 aa, Artificial Sequence
  MART peptide
SLLQHLIGL

SEQ ID NO: 11 - protein, 9 aa, Artificial Sequence
  Myeloblastin peptide
VLQELNVTV

SEQ ID NO: 12 - protein, 11 aa, Artificial Sequence
  LB-ADIR-1F peptide
SVAPALALFPA

SEQ ID NO: 13 - protein, 9 aa, Artificial Sequence
  MTG8b peptide
FLKANLPLL

SEQ ID NO: 14 - protein, 9 aa, Artificial Sequence
  Foxp3 peptide
KLSAMQAHL

SEQ ID NO: 15 - protein, 9 aa, Artificial Sequence
  Foxp3 peptide
LQLPTLPLV

SEQ ID NO: 16 - protein, 9 aa, Artificial Sequence
  HA-1/A2 peptide
VLHDDLLEA

SEQ ID NO: 17 - protein, 9 aa, Artificial Sequence
  Foxp3 peptide
VFEEPEDFL

SEQ ID NO: 18 - protein, 9 aa, Artificial Sequence
  Nucleophosmin peptide
AIQDLCLAV

SEQ ID NO: 19 - protein, 9 aa, Artificial Sequence
  Nucleophosmin peptide
AIQDLCVAV

SEQ ID NO: 20 - protein, 10 aa, Artificial Sequence
  PRAME peptide
ALYVDSLFFL

SEQ ID NO: 21 - protein, 9 aa, Artificial Sequence
  NY-ESO-1 peptide
SLLMWITQV

SEQ ID NO: 22 - protein, 8 aa, Artificial Sequence
  WT1 peptide
RMFNAPYL

SEQ ID NO: 23 - protein, 9 aa, Artificial Sequence
  HTLV-1 Tax peptide
LLFGYPVYV

SEQ ID NO: 24 - protein, 10 aa, Artificial Sequence
  HCV peptide
KLVALGINAV

SEQ ID NO: 25 - protein, 9 aa, Artificial Sequence
  EBV-BLMF1 peptide
GLCTLVAML

SEQ ID NO: 26 - protein, 9 aa, Artificial Sequence
  HBV-SAg peptide
WLSLLVPFV

SEQ ID NO: 27 - protein, 9 aa, Artificial Sequence
  EBV-BRLF1
YVLDHLIVV

SEQ ID NO: 28 - protein, 9 aa, Artificial Sequence
  Human polyomavirus 2 peptide
SITEVECFL

SEQ ID NO: 29 - protein, 9 aa, Artificial Sequence
  HBV peptide
FLLSLGIHL

SEQ ID NO: 30 - protein, 9 aa, Artificial Sequence
  Flu-M1 peptide
GILGFVFTL

SEQ ID NO: 31 - protein, 9 aa, Artificial Sequence
  HIV gag peptide
SLFNTVATL

SEQ ID NO: 32 - protein, 9 aa, Artificial Sequence
  AdV11 hexon peptide
YLLFEVFDV

SEQ ID NO: 33 - protein, 9 aa, Artificial Sequence
  AdV11 hexon peptide
LLFEVFDVV

SEQ ID NO: 34 - protein, 9 aa, Artificial Sequence
  AdV11 hexon peptide
YVLFEVFDV

SEQ ID NO: 35 - protein, 9 aa, Artificial Sequence
  EBV BALF4 peptide
FLDKGTYTL

SEQ ID NO: 36 - protein, 9 aa, Artificial Sequence
  EBV-LMP1-2 peptide
YLQQNWWTL

SEQ ID NO: 37 - protein, 9 aa, Artificial Sequence
  EBV-LMP1-1 peptide
YLLEMLWRL

SEQ ID NO: 38 - protein, 9 aa, Artificial Sequence
  EBV-LMP1-2 peptide
FLYALALLL

SEQ ID NO: 39 - protein, 9 aa, Artificial Sequence
  CMV-IE1 peptide
VLEETSVML

SEQ ID NO: 40 - protein, 9 aa, Artificial Sequence
  HIV gag peptide
TLNAWVKVV

SEQ ID NO: 41 - protein, 9 aa, Artificial Sequence
  Influenza NS1 peptide
AIMDKNIIL

SEQ ID NO: 42 - protein, 9 aa, Artificial Sequence
  M. tuberculosis Ag85A peptide
KLIANNTRV

SEQ ID NO: 43 - protein, 9 aa, Artificial Sequence
  Varicella-zoster IE62 593-601 peptide
ALWALPHAA

SEQ ID NO: 44 - protein, 9 aa, Artificial Sequence
  CMV-pp65 peptide
NLVPMVATV

SEQ ID NO: 45 - protein, 9 aa, Artificial Sequence
  Influenza A peptide
FMYSDFHFI

SEQ ID NO: 46 - protein, 8 aa, Artificial Sequence
  Rota-VP3 peptide
YLLPGWKL

SEQ ID NO: 47 - protein, 9 aa, Artificial Sequence
  Flu-PB1 peptide
NMLSTVLGV

SEQ ID NO: 48 - protein, 11 aa, Artificial Sequence
  Rota-VP1 peptide
SLMDPAILTSL

SEQ ID NO: 49 - protein, 9 aa, Artificial Sequence
  Rota-VP6 peptide
TLLANVTAV

SEQ ID NO: 50 - protein, 11 aa, Artificial Sequence
  CMV-IE1-2 peptide
FMDILTTCVET

SEQ ID NO: 51 - protein, 9 aa, Artificial Sequence
  CMV-pp65-2 peptide
QMWQARLTV

SEQ ID NO: 52 - protein, 9 aa, Artificial Sequence
  Rota-VP2-1 peptide
SLISGMWLL

SEQ ID NO: 53 - protein, 9 aa, Artificial Sequence
  Rota-VP7-1 peptide
LLNYILKSV

SEQ ID NO: 54 - protein, 9 aa, Artificial Sequence
  CMV-pp65-3 peptide
LMNGQQIFL

SEQ ID NO: 55 - protein, 9 aa, Artificial Sequence
  Rota-NSP1 peptide
FLDSEPHLL

SEQ ID NO: 56 - protein, 10 aa, Artificial Sequence
  Proinsulin precursor 15-24 peptide
ALWGPDPAAA

SEQ ID NO: 57 - protein, 9 aa, Artificial Sequence
  EBV BMRF1 peptide
TLDYKPLSV

SEQ ID NO: 58 - protein, 9 aa, Artificial Sequence
  EBV-LMP2A peptide
CLGGLLTMV

SEQ ID NO: 59 - protein, 9 aa, Artificial Sequence
  AdV 11 Hexon (37-45) peptide
TYFNLGNKF

SEQ ID NO: 60 - protein, 9 aa, Artificial Sequence
  AdV 11 Hexon (696-704) peptide
VYSGSIPYL

SEQ ID NO: 61 - protein, 9 aa, Artificial Sequence
  AdV 5 Hexon (37-45) peptide
TYFSLNNKF

SEQ ID NO: 62 - protein, 9 aa, Artificial Sequence
  EBV BMLF1 (320-328) peptide
DYNFVKQLF

SEQ ID NO: 63 - protein, 9 aa, Artificial Sequence
  EBV BRLF1 (198-206)
TYPVLEEMF

SEQ ID NO: 64 - protein, 9 aa, Artificial Sequence
  EBV EBNA3A (246-254) peptide
RYSIFFDYM

SEQ ID NO: 65 - protein, 9 aa, Artificial Sequence
  EBV EBMA3B (217-225) peptide
TYSAGIVQI

SEQ ID NO: 66 - protein, 9 aa, Artificial Sequence
  EBV LMP2 (222-230) peptide
IYVLVMLVL

SEQ ID NO: 67 - protein, 9 aa, Artificial Sequence
  EBV LMP2 (131-139) peptide
PYLFWLAAI

SEQ ID NO: 68 - protein, 9 aa, Artificial Sequence
  EBV LMP2 (419-427) peptide
TYGPVFMSL

SEQ ID NO: 69 - protein, 9 aa, Artificial Sequence
  EBV LMP2 (419-427) peptide
TYGPVFMCL

SEQ ID NO: 70 - protein, 9 aa, Artificial Sequence
  HBV core (117-125) peptide
EYLVSFGVW

SEQ ID NO: 71 - protein, 9 aa, Artificial Sequence
  HBV pol (756-764) peptide
KYTSFPWLL

SEQ ID NO: 72 - protein, 9 aa, Artificial Sequence
  HCMV pp65 (341-349) peptide
QYDPVAALF

SEQ ID NO: 73 - protein, 9 aa, Artificial Sequence
  HCV E2 (717-725) peptide
EYVLLLFLL

SEQ ID NO: 74 - protein, 9 aa, Artificial Sequence
  HHV-6B U54 (267-275) peptide
PFHCSFHTI

SEQ ID NO: 75 - protein, 9 aa, Artificial Sequence
  HIV env gp160 (584-592) peptide
RYLRDQQLL

SEQ ID NO: 76 - protein, 9 aa, Artificial Sequence
  HIV env (67-75) peptide
RYLKDQQLL

SEQ ID NO: 77 - protein, 8 aa, Artificial Sequence
  HIV nef (134-141) peptide
RYPLTFGW

SEQ ID NO: 78 - protein, 9 aa, Artificial Sequence
  HPV16 E6 (49-57) peptide
VYDFAFRDL

SEQ ID NO: 79 - protein, 9 aa, Artificial Sequence
  HTLV-1 Env (43788) peptide
FFQFCPLIF

SEQ ID NO: 80 - protein, 9 aa, Artificial Sequence
  HTLV-1 Tax (43819) peptide
LFGYPVYVF

SEQ ID NO: 81 - protein, 9 aa, Artificial Sequence
  HTLV-1 Tax (187-195) peptide
PYKRIEELL

SEQ ID NO: 82 - protein, 9 aa, Artificial Sequence
  HTLV-1 Tax (301-309) peptide
SFHSLHLLF

SEQ ID NO: 83 - protein, 9 aa, Artificial Sequence
  Influenza PA (130-138) peptide
YYLEKANKI

SEQ ID NO: 84 - protein, 9 aa, Artificial Sequence
  Influenza PB1 (216-224)
SYLIRALTL

SEQ ID NO: 85 - protein, 9 aa, Artificial Sequence
  Influenza PB1 (430-438) peptide
RYTKTTYWW

SEQ ID NO: 86 - protein, 9 aa, Artificial Sequence
  Influenza PB1 (482-490) peptide
SYINRTGTF

SEQ ID NO: 87 - protein, 8 aa, Artificial Sequence
  Influenza PB1 (498-505) peptide
RYGFVANF

SEQ ID NO: 88 - protein, 9 aa, Artificial Sequence
  Influenza PB2 (549-557) peptide
TYQWIIRNW

SEQ ID NO: 89 - protein, 12 aa, Artificial Sequence
  CDR3 alpha peptide
CATVGTASKLTF

SEQ ID NO: 90 - protein, 11 aa, Artificial Sequence
  CDR3 alpha peptide
CARNTGNQFYF

SEQ ID NO: 91 - protein, 10 aa, Artificial Sequence
  CDR3 alpha peptide
CVVGYGQFYF

SEQ ID NO: 92 - protein, 14 aa, Artificial Sequence
  CDR3 alpha peptide
CAGPMKTSYDKVIF

SEQ ID NO: 93 - protein, 14 aa, Artificial Sequence
  CDR3 alpha peptide
CAASRKGSNYKLTF

SEQ ID NO: 94 - protein, 11 aa, Artificial Sequence
  CDR3 alpha peptide
CAVRWGGKLSF

SEQ ID NO: 95 - protein, 16 aa, Artificial Sequence
  CDR3 alpha peptide
CAEIPNYGGSQGNLIF

SEQ ID NO: 96 - protein, 11 aa, Artificial Sequence
  CDR3 alpha peptide
CAESSASKIIF

SEQ ID NO: 97 - protein, 16 aa, Artificial Sequence
  CDR3 alpha peptide
CAVRDRWSGGYQKVTF

SEQ ID NO: 98 - protein, 14 aa, Artificial Sequence
  CDR3 alpha peptide
CAVRVSGGYNKLIF

SEQ ID NO: 99 - protein, 15 aa, Artificial Sequence
  CDR3 alpha peptide
CAVTLNNNAGNMLTF

SEQ ID NO: 100 - protein, 16 aa, Artificial Sequence
  CDR3 alpha peptide
CALSPRTQGGSEKLVF

SEQ ID NO: 101 - protein, 12 aa, Artificial Sequence
  CDR3 beta peptide
CASSLWLNEQFF

SEQ ID NO: 102 - protein, 15 aa, Artificial Sequence
  CDR3 beta peptide
CASSPKTGASYGYTF

SEQ ID NO: 103 - protein, 13 aa, Artificial Sequence
  CDR3 beta peptide
CASSFVSFDEQFF

SEQ ID NO: 104 - protein, 12 aa, Artificial Sequence
  CDR3 beta peptide
CASSSAYYGYTF

SEQ ID NO: 105 - protein, 15 aa, Artificial Sequence
  CDR3 beta peptide
CASSADSYGANVLTF

SEQ ID NO: 106 - protein, 14 aa, Artificial Sequence
  CDR3 beta peptide
CSVDPGHTGEKLFF

SEQ ID NO: 107 - protein, 14 aa, Artificial Sequence
  CDR3 beta peptide
CASSLVGGRHGYTF

SEQ ID NO: 108 - protein, 17 aa, Artificial Sequence
  CDR3 beta peptide
CASSHDPTWGPGNTIYF

SEQ ID NO: 109 - protein, 14 aa, Artificial Sequence
  CDR3 beta peptide
CASSFGQGSSPLHF

SEQ ID NO: 110 - protein, 14 aa, Artificial Sequence
  CDR3 beta peptide
CASSLETVNTEAFF

SEQ ID NO: 111 - protein, 15 aa, Artificial Sequence
  CDR3 beta peptide
CASSSFYDSNEKLFF

SEQ ID NO: 112 - protein, 17 aa, Artificial Sequence
  CDR3 beta peptide
CASSLASPGHFTGELFF

SEQ ID NO: 113 - protein, 9 aa, Artificial Sequence
  SARS-CoV-2 peptide
RLDKVEAEV

SEQ ID NO: 114 - protein, 10 aa, Artificial Sequence
  SARS-CoV-2 peptide
KLPDDFTGCV

SEQ ID NO: 115 - protein, 10 aa, Artificial Sequence
  SARS-CoV-2 peptide
KWPWYIWLGF

SEQ ID NO: 116 - protein, 10 aa, Artificial Sequence
  SARS-CoV-2 peptide
FCLEASFNYL

SEQ ID NO: 117 - protein, 9 aa, Artificial Sequence
  SARS-CoV-2 peptide
MLAKALRKV

SEQ ID NO: 118 - protein, 10 aa, Artificial Sequence
  SARS-CoV-2 peptide
YLQPRTFLLK

SEQ ID NO: 119 - protein, 9 aa, Artificial Sequence
  SARS-CoV-2 peptide
KQIYKTPPI

SEQ ID NO: 120 - protein, 9 aa, Artificial Sequence
  SARS-CoV-2 peptide
MLAKALRKV

SEQ ID NO: 121 - protein, 10 aa, Artificial Sequence
  SARS-CoV-2 peptide
RLITGRLQSL

SEQ ID NO: 122 - protein, 9 aa, Artificial Sequence
  SARS-CoV-2 peptide
MLAKALRKV

SEQ ID NO: 123 - protein, 10 aa, Artificial Sequence
  SARS-CoV-2 peptide
RLITGRLQSL

SEQ ID NO: 124 - protein, 9 aa, Artificial Sequence
  SARS-CoV-2 peptide
KQIYKTPPI

SEQ ID NO: 125 - protein, 11 aa, Artificial Sequence
  SARS-CoV-2 peptide
FPQSAPHGVVF

SEQ ID NO: 126 - protein, 9 aa, Artificial Sequence
  SARS-CoV-2 peptide
LPPAYTNSF

SEQ ID NO: 127 - protein, 10 aa, Artificial Sequence
  SARS-CoV-2 peptide
RARSVASQSI

SEQ ID NO: 128 - protein, 10 aa, Artificial Sequence
  SARS-CoV-2 peptide
YPDKVFRSSV

SEQ ID NO: 129 - protein, 9 aa, Artificial Sequence
  SARS-CoV-2 peptide
SPRRARSVA

SEQ ID NO: 130 - protein, 10 aa, Artificial Sequence
  SARS-CoV-2 peptide
RLITGRLQSL

SEQ ID NO: 131 - protein, 9 aa, Artificial Sequence
  SARS-CoV-2 peptide
LLFNKVTLA

SEQ ID NO: 132 - protein, 10 aa, Artificial Sequence
  SARS-CoV-2 peptide
RLITGRLQSL

SEQ ID NO: 133 - protein, 9 aa, Artificial Sequence
  SARS-CoV-2 peptide
LLFNKVTLA

SEQ ID NO: 134 - protein, 10 aa, Artificial Sequence
  antigen peptide
SLHAHGLSYK

SEQ ID NO: 135 - protein, 16 aa, Artificial Sequence
  Placeholder peptide
SALSEGATPQDLNTML

SEQ ID NO: 136 - protein, 15 aa, Artificial Sequence
  Purification tag
GLNDIFEAQKIEWHE

SEQ ID NO: 137 - protein, 15 aa, Artificial Sequence
  Glycine-serine linker
GGGGSGGGGSGGGGS

SEQ ID NO: 138 - protein, 15 aa, Artificial Sequence
  Glycine-serine linker
GCGGSGGGGSGGGGS

SEQ ID NO: 139 - protein, 15 aa, Artificial Sequence
  Glycine-serine linker
GCGASGGGGSGGGGS

SEQ ID NO: 140 - protein, 20 aa, Artificial Sequence
  Glycine-serine linker
GGGGSGGGGSGGGGSGGGGS

SEQ ID NO: 141 - protein, 5 aa, Artificial Sequence
  Linker GS moiety
GGGGS

SEQ ID NO: 142 - protein, 5 aa, Artificial Sequence
  Linker GS moiety
GCGGS

SEQ ID NO: 143 - protein, 5 aa, Artificial Sequence
  Linker GS moiety
GGCGS

SEQ ID NO: 144 - protein, 5 aa, Artificial Sequence
  Linker GS moiety
GCGAS

SEQ ID NO: 145 - protein, 11 aa, Artificial Sequence
  SARS-CoV-2 spike protein peptide
VLLPLVSSQCV

SEQ ID NO: 146 - protein, 9 aa, Artificial Sequence
  SARS-CoV-2 peptide
FQFCNDPFL

SEQ ID NO: 147 - protein, 11 aa, Artificial Sequence
  SARS-CoV-2 peptide
FQFCNDPFLGV

SEQ ID NO: 148 - protein, 10 aa, Artificial Sequence
  SARS-CoV-2 peptide
FQFCNDPFLG

SEQ ID NO: 149 - protein, 9 aa, Artificial Sequence
  SARS-CoV-2 peptide
KIYSKHTPI

SEQ ID NO: 150 - protein, 10 aa, Artificial Sequence
  SARS-CoV-2 peptide
TLLALHRSYL

SEQ ID NO: 151 - protein, 9 aa, Artificial Sequence
  SARS-CoV-2 peptide
YLQPRTFLL

SEQ ID NO: 152 - protein, 10 aa, Artificial Sequence
  SARS-CoV-2 peptide
YLQPRTFLLK

SEQ ID NO: 153 - protein, 10 aa, Artificial Sequence
  SARS-CoV-2 peptide
VLYNSASFST

SEQ ID NO: 154 - protein, 9 aa, Artificial Sequence
  SARS-CoV-2 peptide
KIADYNYKL

SEQ ID NO: 155 - protein, 10 aa, Artificial Sequence
  SARS-CoV-2 peptide
KLPDDFTGCV

SEQ ID NO: 156 - protein, 9 aa, Artificial Sequence
  SARS-CoV-2 peptide
ELLHAPATV

SEQ ID NO: 157 - protein, 9 aa, Artificial Sequence
  SARS-CoV-2 peptide
YQDVNCTEV

SEQ ID NO: 158 - protein, 9 aa, Artificial Sequence
  SARS-CoV-2 peptide
SIIAYTMSL

SEQ ID NO: 159 - protein, 9 aa, Artificial Sequence
  SARS-CoV-2 peptide
FTISVTTEI

SEQ ID NO: 160 - protein, 10 aa, Artificial Sequence
  SARS-CoV-2 peptide
FTISVTTEIL

SEQ ID NO: 161 - protein, 9 aa, Artificial Sequence
  SARS-CoV-2 peptide
SVTTEILPV

SEQ ID NO: 162 - protein, 9 aa, Artificial Sequence
  SARS-CoV-2 peptide
KQIYKTPPI

SEQ ID NO: 163 - protein, 9 aa, Artificial Sequence
  SARS-CoV-2 peptide
LFFNKVTLA

SEQ ID NO: 164 - protein, 9 aa, Artificial Sequence
  SARS-CoV-2 peptide
MIAQYTSAL

SEQ ID NO: 165 - protein, 10 aa, Artificial Sequence
  SARS-CoV-2 peptide
MIAQYTSALL

SEQ ID NO: 166 - protein, 10 aa, Artificial Sequence
  SARS-CoV-2 peptide
ALQIPFAMQM

SEQ ID NO: 167 - protein, 10 aa, Artificial Sequence
  SARS-CoV-2 peptide
MQMAYRFNGI

SEQ ID NO: 168 - protein, 10 aa, Artificial Sequence
  SARS-CoV-2 peptide
KLIANQFNSA

SEQ ID NO: 169 - protein, 9 aa, Artificial Sequence
  SARS-CoV-2 peptide
SLSSTASAL

SEQ ID NO: 170 - protein, 9 aa, Artificial Sequence
  SARS-CoV-2 peptide
RLDKVEAEV

SEQ ID NO: 171 - protein, 10 aa, Artificial Sequence
  SARS-CoV-2 peptide
RLITGRLQSL

SEQ ID NO: 172 - protein, 9 aa, Artificial Sequence
  SARS-CoV-2 peptide
HLMSFPQSA

SEQ ID NO: 173 - protein, 9 aa, Artificial Sequence
  SARS-CoV-2 peptide
FLHVTYVPA

SEQ ID NO: 174 - protein, 10 aa, Artificial Sequence
  SARS-CoV-2 peptide
FVSNGTHWFV

SEQ ID NO: 175 - protein, 9 aa, Artificial Sequence
  SARS-CoV-2 peptide
LPPAYTNSF

SEQ ID NO: 176 - protein, 10 aa, Artificial Sequence
  SARS-CoV-2 peptide
YPDKVFRSSV

SEQ ID NO: 177 - protein, 11 aa, Artificial Sequence
  SARS-CoV-2 peptide
YPDKVFRSSVL

SEQ ID NO: 178 - protein, 9 aa, Artificial Sequence
  SARS-CoV-2 peptide
TPINLVRDL

SEQ ID NO: 179 - protein, 11 aa, Artificial Sequence
  SARS-CoV-2 peptide
LPQGFSALEPL

SEQ ID NO: 180 - protein, 9 aa, Artificial Sequence
  SARS-CoV-2 peptide
APGQTGKIA

SEQ ID NO: 181 - protein, 11 aa, Artificial Sequence
  SARS-CoV-2 peptide
KPFERDISTEI

SEQ ID NO: 182 - protein, 10 aa, Artificial Sequence
  SARS-CoV-2 peptide
TPCSFGGVSV

SEQ ID NO: 183 - protein, 10 aa, Artificial Sequence
  SARS-CoV-2 peptide
VPVAIHADQL

SEQ ID NO: 184 - protein, 9 aa, Artificial Sequence
  SARS-CoV-2 peptide
SPRRARSVA

SEQ ID NO: 185 - protein, 10 aa, Artificial Sequence
  SARS-CoV-2 peptide
SPRRARSVAS

SEQ ID NO: 186 - protein, 11 aa, Artificial Sequence
  SARS-CoV-2 peptide
SPRRARSVASQ

SEQ ID NO: 187 - protein, 10 aa, Artificial Sequence
  SARS-CoV-2 peptide
RARSVASQSI

SEQ ID NO: 188 - protein, 9 aa, Artificial Sequence
  SARS-CoV-2 peptide
SIIAYTMSL

SEQ ID NO: 189 - protein, 10 aa, Artificial Sequence
  SARS-CoV-2 peptide
AIPTNFTISV

SEQ ID NO: 190 - protein, 9 aa, Artificial Sequence
  SARS-CoV-2 peptide
IPTNFTISV

SEQ ID NO: 191 - protein, 10 aa, Artificial Sequence
  SARS-CoV-2 peptide
LPVSMTKTSV

SEQ ID NO: 192 - protein, 9 aa, Artificial Sequence
  SARS-CoV-2 peptide
MIAQYTSAL

SEQ ID NO: 193 - protein, 10 aa, Artificial Sequence
  SARS-CoV-2 peptide
MIAQYTSALL

SEQ ID NO: 194 - protein, 10 aa, Artificial Sequence
  SARS-CoV-2 peptide
FPQSAPHGVV

SEQ ID NO: 195 - protein, 9 aa, Artificial Sequence
  SARS-CoV-2 peptide
FPQSAPHGV

SEQ ID NO: 196 - protein, 11 aa, Artificial Sequence
  SARS-CoV-2 peptide
FPQSAPHGVVF

SEQ ID NO: 197 - protein, 9 aa, Artificial Sequence
  SARS-Co-V-2 peptide
YYHKNNKSW

SEQ ID NO: 198 - protein, 11 aa, Artificial Sequence
  AARS-CoV-2 peptide
RVYSSANNCTF

SEQ ID NO: 199 - protein, 10 aa, Artificial Sequence
  SARS-CoV-2 peptide
VYSSANNCTF

SEQ ID NO: 200 - protein, 11 aa, Artificial Sequence
  SARS-CoV-2 peptide
VYSSANNCTFE

SEQ ID NO: 201 - protein, 9 aa, Artificial Sequence
  SARS-CoV-2 peptide
TFEYVSQPF

SEQ ID NO: 202 - protein, 9 aa, Artificial Sequence
  SARS-CoV-2 peptide
EYVSQPFLM

SEQ ID NO: 203 - protein, 11 aa, Artificial Sequence
  SARS-CoV-2 peptide
YYVGYLQPRTF

SEQ ID NO: 204 - protein, 10 aa, Artificial Sequence
  SARS-CoV-2 peptide
GYLQPRTFLL

SEQ ID NO: 205 - protein, 9 aa, Artificial Sequence
  SARS-CoV-2 peptide
YLQPRTFLL

SEQ ID NO: 206 - protein, 10 aa, Artificial Sequence
  SARS-CoV-2 peptide
LYNSASFSTF

SEQ ID NO: 207 - protein, 11 aa, Artificial Sequence
  SARS-CoV-2 peptide
LYNSASFSTFK

SEQ ID NO: 208 - protein, 9 aa, Artificial Sequence
  SARS-CoV-2 peptide
YNSASFSTF

SEQ ID NO: 209 - protein, 10 aa, Artificial Sequence
  SARS-CoV-2 peptide
CFTNVYADSF

SEQ ID NO: 210 - protein, 10 aa, Artificial Sequence
  SARS-CoV-2 peptide
GNYNYLYRLF

SEQ ID NO: 211 - protein, 9 aa, Artificial Sequence
  SARS-CoV-2 peptide
NYNYLYRLF

SEQ ID NO: 212 - protein, 10 aa, Artificial Sequence
  SARS-CoV-2 peptide
NYNYLYRLFR

SEQ ID NO: 213 - protein, 11 aa, Artificial Sequence
  SARS-CoV-2 peptide
NCYFPLQSYGF

SEQ ID NO: 214 - protein, 11 aa, Artificial Sequence
  SARS-CoV-2 peptide
CYFPLQSYGFQ

SEQ ID NO: 215 - protein, 10 aa, Artificial Sequence
  SARS-CoV-2 peptide
YFPLQSYGFQ

SEQ ID NO: 216 - protein, 11 aa, Artificial Sequence
  SARS-CoV-2 peptide
RVYSTGSNVFQ

SEQ ID NO: 217 - protein, 9 aa, Artificial Sequence
  SARS-CoV-2 peptide
VYSTGSNVF

SEQ ID NO: 218 - protein, 10 aa, Artificial Sequence
  SARS-CoV-2 peptide
VYSTGSNVFQ

SEQ ID NO: 219 - protein, 10 aa, Artificial Sequence
  SARS-CoV-2 peptide
IYKTPPIKDF

SEQ ID NO: 220 - protein, 11 aa, Artificial Sequence
  SARS-CoV-2 peptide
IPFAMQMAYRF

SEQ ID NO: 221 - protein, 9 aa, Artificial Sequence
  SARS-CoV-2 peptide
FAMQMAYRF

SEQ ID NO: 222 - protein, 10 aa, Artificial Sequence
  SARS-CoV-2 peptide
TYVPAQEKNF

SEQ ID NO: 223 - protein, 10 aa, Artificial Sequence
  SARS-CoV-2 peptide
VFVSNGTHWF

SEQ ID NO: 224 - protein, 10 aa, Artificial Sequence
  SARS-CoV-2 peptide
KYEQYIKWPW

SEQ ID NO: 225 - protein, 11 aa, Artificial Sequence
  SARS-CoV-2 peptide
YEQYIKWPWYI

SEQ ID NO: 226 - protein, 10 aa, Artificial Sequence
  SARS-CoV-2 peptide
EQYIKWPWYI

SEQ ID NO: 227 - protein, 11 aa, Artificial Sequence
  SARS-CoV-2 peptide
EQYIKWPWYIW

SEQ ID NO: 228 - protein, 9 aa, Artificial Sequence
  SARS-CoV-2 peptide
QYIKWPWYI

SEQ ID NO: 229 - protein, 11 aa, Artificial Sequence
  SARS-CoV-2 peptide
QYIKWPWYIWL

SEQ ID NO: 230 - protein, 11 aa, Artificial Sequence
  SARS-CoV-2 peptide
IKWPWYIWLGF

SEQ ID NO: 231 - protein, 10 aa, Artificial Sequence
  SARS-CoV-2 peptide
KWPWYIWLGF

SEQ ID NO: 232 - protein, 11 aa, Artificial Sequence
  SARS-CoV-2 peptide
KWPWYIWLGFI

SEQ ID NO: 233 - protein, 9 aa, Artificial Sequence
  SARS-CoV-2 peptide
FGDDTVIEV

SEQ ID NO: 234 - protein, 9 aa, Artificial Sequence
  SARS-CoV-2 peptide
KSVNITFEL

SEQ ID NO: 235 - protein, 9 aa, Artificial Sequence
  SARS-CoV-2 peptide
YTVELGTEV

SEQ ID NO: 236 - protein, 9 aa, Artificial Sequence
  SARS-CoV-2 peptide
AVIKTLQPV

SEQ ID NO: 237 - protein, 9 aa, Artificial Sequence
  SARS-CoV-2 peptide
TLQPVSELL

SEQ ID NO: 238 - protein, 11 aa, Artificial Sequence
  SARS-CoV-2 peptide
YLFDESGEFKL

SEQ ID NO: 239 - protein, 9 aa, Artificial Sequence
  SARS-CoV-2 peptide
YLFDESGEF

SEQ ID NO: 240 - protein, 9 aa, Artificial Sequence
  SARS-CoV-2 peptide
KLASHMYCS

SEQ ID NO: 241 - protein, 10 aa, Artificial Sequence
  SARS-CoV-2 peptide
WLDDDSQQTV

SEQ ID NO: 242 - protein, 9 aa, Artificial Sequence
  SARS-CoV-2 peptide
TTIQTIVEV

SEQ ID NO: 243 - protein, 9 aa, Artificial Sequence
  SARS-CoV-2 peptide
TIVEVQPQL

SEQ ID NO: 244 - protein, 9 aa, Artificial Sequence
  SARS-CoV-2 peptide
MQVESDDYI

SEQ ID NO: 245 - protein, 9 aa, Artificial Sequence
  SARS-CoV-2 peptide
VLLAPLLSA

SEQ ID NO: 246 - protein, 10 aa, Artificial Sequence
  SARS-CoV-2 peptide
LLAPLLSAGI

SEQ ID NO: 247 - protein, 9 aa, Artificial Sequence
  SARS-CoV-2 peptide
LLSAGIFGA

SEQ ID NO: 248 - protein, 10 aa, Artificial Sequence
  SARS-CoV-2 peptide
LLSAGIFGAD

SEQ ID NO: 249 - protein, 9 aa, Artificial Sequence
  SARS-CoV-2 peptide
YLAVFDKNL

SEQ ID NO: 250 - protein, 11 aa, Artificial Sequence
  SARS-CoV-2 peptide
NLYDKLVSSFL

SEQ ID NO: 251 - protein, 9 aa, Artificial Sequence
  SARS-CoV-2 peptide
KLVSSFLEM

SEQ ID NO: 252 - protein, 10 aa, Artificial Sequence
  SARS-CoV-2 peptide
KIAEIPKEEV

SEQ ID NO: 253 - protein, 9 aa, Artificial Sequence
  SARS-CoV-2 peptide
FITESKPSV

SEQ ID NO: 254 - protein, 11 aa, Artificial Sequence
  SARS-CoV-2 peptide
KFLTENLLLYI

SEQ ID NO: 255 - protein, 10 aa, Artificial Sequence
  SARS-CoV-2 peptide
FLTENLLLYI

SEQ ID NO: 256 - protein, 11 aa, Artificial Sequence
  SARS-CoV-2 peptide
FLTENLLLYID

SEQ ID NO: 257 - protein, 10 aa, Artificial Sequence
  SARS-CoV-2 peptide
LLYIDINGNL

SEQ ID NO: 258 - protein, 9 aa, Artificial Sequence
  SARS-CoV-2 peptide
FLKKDAPYI

SEQ ID NO: 259 - protein, 10 aa, Artificial Sequence
  SARS-CoV-2 peptide
FLKKDAPYIV

SEQ ID NO: 260 - protein, 9 aa, Artificial Sequence
  SARS-CoV-2 peptide
MLAKALRKV

SEQ ID NO: 261 - protein, 9 aa, Artificial Sequence
  SARS-CoV-2 peptide
KLMPVCVET

SEQ ID NO: 262 - protein, 11 aa, Artificial Sequence
  SARS-CoV-2 peptide
KLMPVCVETKA

SEQ ID NO: 263 - protein, 10 aa, Artificial Sequence
  SARS-CoV-2 peptide
KLMPVCVETK

SEQ ID NO: 264 - protein, 8 aa, Artificial Sequence
  SARS-CoV-2 peptide
SLNTLNDL

SEQ ID NO: 265 - protein, 10 aa, Artificial Sequence
  SARS-CoV-2 peptide
TLVTMPLGYV

SEQ ID NO: 266 - protein, 9 aa, Artificial Sequence
  SARS-CoV-2 peptide
RTIKVFTTV

SEQ ID NO: 267 - protein, 9 aa, Artificial Sequence
  SARS-CoV-2 peptide
FLGRYMSAL

SEQ ID NO: 268 - protein, 10 aa, Artificial Sequence
  SARS-CoV-2 peptide
ALLTLQQIEL

SEQ ID NO: 269 - protein, 9 aa, Artificial Sequence
  SARS-CoV-2 peptide
LLTLQQIEL

SEQ ID NO: 270 - protein, 10 aa, Artificial Sequence
  SARS-CoV-2 peptide
YLVQQESPFV

SEQ ID NO: 271 - protein, 11 aa, Artificial Sequence
  SARS-CoV-2 peptide
YLVQQESPFVM

SEQ ID NO: 272 - protein, 10 aa, Artificial Sequence
  SARS-CoV-2 peptide
PFVMMSAPPA

SEQ ID NO: 273 - protein, 9 aa, Artificial Sequence
  SARS-CoV-2 peptide
FVMMSAPPA

SEQ ID NO: 274 - protein, 9 aa, Artificial Sequence
  SARS-CoV-2 peptide
FVCDNIKFA

SEQ ID NO: 275 - protein, 11 aa, Artificial Sequence
  SARS-CoV-2 peptide
KLLHKPIVWHV

SEQ ID NO: 276 - protein, 9 aa, Artificial Sequence
  SARS-CoV-2 peptide
YVDNSSLTI

SEQ ID NO: 277 - protein, 10 aa, Artificial Sequence
  SARS-CoV-2 peptide
TLATHGLAAV

SEQ ID NO: 278 - protein, 9 aa, Artificial Sequence
  SARS-CoV-2 peptide
FLNKVVSTT

SEQ ID NO: 279 - protein, 9 aa, Artificial Sequence
  SARS-CoV-2 peptide
LLLQLCTFT

SEQ ID NO: 280 - protein, 10 aa, Artificial Sequence
  SARS-CoV-2 peptide
FCLEASFNYL

SEQ ID NO: 281 - protein, 10 aa, Artificial Sequence
  SARS-CoV-2 peptide
LLLSVCLGSL

SEQ ID NO: 282 - protein, 10 aa, Artificial Sequence
  SARS-CoV-2 peptide
GSLIYSTAAL

SEQ ID NO: 283 - protein, 9 aa, Artificial Sequence
  SARS-CoV-2 peptide
SLIYSTAAL

SEQ ID NO: 284 - protein, 10 aa, Artificial Sequence
  SARS-CoV-2 peptide
VLMSNLGMPS

SEQ ID NO: 285 - protein, 9 aa, Artificial Sequence
  SARS-CoV-2 peptide
SLETIQITI

SEQ ID NO: 286 - protein, 10 aa, Artificial Sequence
  SARS-CoV-2 peptide
FGLVAEWFLA

SEQ ID NO: 287 - protein, 9 aa, Artificial Sequence
  SARS-CoV-2 peptide
GLVAEWFLA

SEQ ID NO: 288 - protein, 11 aa, Artificial Sequence
  SARS-CoV-2 peptide
GLVAEWFLAYI

SEQ ID NO: 289 - protein, 10 aa, Artificial Sequence
  SARS-CoV-2 peptide
YILFTRFFYV

SEQ ID NO: 290 - protein, 10 aa, Artificial Sequence
  SARS-CoV-2 peptide
MQLFFSYFAV

SEQ ID NO: 291 - protein, 9 aa, Artificial Sequence
  SARS-CoV-2 peptide
MQLFFSYFA

SEQ ID NO: 292 - protein, 11 aa, Artificial Sequence
  SARS-CoV-2 peptide
LVQMAPISAMV

SEQ ID NO: 293 - protein, 10 aa, Artificial Sequence
  SARS-CoV-2 peptide
VQMAPISAMV

SEQ ID NO: 294 - protein, 9 aa, Artificial Sequence
  SARS-CoV-2 peptide
YVWKSYVHV

SEQ ID NO: 295 - protein, 10 aa, Artificial Sequence
  SARS-CoV-2 peptide
YVWKSYVHVV

SEQ ID NO: 296 - protein, 10 aa, Artificial Sequence
  SARS-CoV-2 peptide
SQLMCQPILL

SEQ ID NO: 297 - protein, 9 aa, Artificial Sequence
  SARS-CoV-2 peptide
QLMCQPILL

SEQ ID NO: 298 - protein, 10 aa, Artificial Sequence
  SARS-CoV-2 peptide
QLMCQPILLL

SEQ ID NO: 299 - protein, 9 aa, Artificial Sequence
  SARS-CoV-2 peptide
LMCQPILLL

SEQ ID NO: 300 - protein, 9 aa, Artificial Sequence
  SARS-CoV-2 peptide
ILLLDQALV

SEQ ID NO: 301 - protein, 11 aa, Artificial Sequence
  SARS-CoV-2 peptide
LLLDQALVSDV

SEQ ID NO: 302 - protein, 10 aa, Artificial Sequence
  SARS-CoV-2 peptide
LLDQALVSDV

SEQ ID NO: 303 - protein, 9 aa, Artificial Sequence
  SARS-CoV-2 peptide
KMFDAYVNT

SEQ ID NO: 304 - protein, 11 aa, Artificial Sequence
  SARS-CoV-2 peptide
YVNTFSSTFNV

SEQ ID NO: 305 - protein, 9 aa, Artificial Sequence
  SARS-CoV-2 peptide
NTFSSTFNV

SEQ ID NO: 306 - protein, 10 aa, Artificial Sequence
  SARS-CoV-2 peptide
SLDNVLSTFI

SEQ ID NO: 307 - protein, 10 aa, Artificial Sequence
  SARS-CoV-2 peptide
KLSHQSDIEV

SEQ ID NO: 308 - protein, 13 aa, Artificial Sequence
  CDR3 alpha peptide
CATEDNAGNMLTF

SEQ ID NO: 309 - protein, 10 aa, Artificial Sequence
  CDR3 alpha peptide
CAVSDDKLIF

SEQ ID NO: 310 - protein, 16 aa, Artificial Sequence
  CDR3 alpha peptide
CAVQAAREYNFNKFYF

SEQ ID NO: 311 - protein, 13 aa, Artificial Sequence
  CDR3 alpha peptide
CLVDNNAGNMLTF

SEQ ID NO: 312 - protein, 15 aa, Artificial Sequence
  CDR3 alpha peptide
CAAQSNMEYGNKLVF

SEQ ID NO: 313 - protein, 13 aa, Artificial Sequence
  CDR3 alpha peptide
CAVNADRDDKIIF

SEQ ID NO: 314 - protein, 14 aa, Artificial Sequence
  CDR3 alpha peptide
CAGHPKTSYDKVIF

SEQ ID NO: 315 - protein, 15 aa, Artificial Sequence
  CDR3 alpha peptide
CALKTIKAAGNKLTF

SEQ ID NO: 316 - protein, 13 aa, Artificial Sequence
  CDR3 alpha peptide
CVVRDGGYNKLIF

SEQ ID NO: 317 - protein, 14 aa, Artificial Sequence
  CDR3 alpha peptide
CAASDDNYGQNFVF

SEQ ID NO: 318 - protein, 15 aa, Artificial Sequence
  CDR3 alpha peptide
CAVLNYGGSQGNLIF

SEQ ID NO: 319 - protein, 11 aa, Artificial Sequence
  CDR3 alpha peptide
CAGNYGQNFVF

SEQ ID NO: 320 - protein, 14 aa, Artificial Sequence
  CDR3 alpha peptide
CAASAGSGTYKYIF

SEQ ID NO: 321 - protein, 13 aa, Artificial Sequence
  CDR3 alpha peptide
CAVSSGGYQKVTF

SEQ ID NO: 322 - protein, 13 aa, Artificial Sequence
  CDR3 alpha peptide
CAFSQGGSEKLVF

SEQ ID NO: 323 - protein, 11 aa, Artificial Sequence
  CDR3 alpha peptide
CAPDSNYQLIW

SEQ ID NO: 324 - protein, 13 aa, Artificial Sequence
  CDR3 alpha peptide
CAGLNQGAQKLVF

SEQ ID NO: 325 - protein, 18 aa, Artificial Sequence
  CDR3 alpha peptide
CAFMEVEGVMNRDDKIIF

SEQ ID NO: 326 - protein, 14 aa, Artificial Sequence
  CDR3 alpha peptide
CAGPIGTSYDKVIF

SEQ ID NO: 327 - protein, 14 aa, Artificial Sequence
  CDR3 alpha peptide
CAFMKLWTGNQFYF

SEQ ID NO: 328 - protein, 11 aa, Artificial Sequence
  CDR3 beta peptide
CASSLGEPQHF

SEQ ID NO: 329 - protein, 13 aa, Artificial Sequence
  CDR3 beta peptide
CASGQGGGTEAFF

SEQ ID NO: 330 - protein, 15 aa, Artificial Sequence
  CDR3 beta peptide
CASSQEGDRVTEAFF

SEQ ID NO: 331 - protein, 12 aa, Artificial Sequence
  CDR3 beta peptide
CASSLRSYEQYF

SEQ ID NO: 332 - protein, 17 aa, Artificial Sequence
  CDR3 beta peptide
CASSSPDRGGRNEKLFF

SEQ ID NO: 333 - protein, 17 aa, Artificial Sequence
  CDR3 beta peptide
CASSLGTSGGAPETQYF

SEQ ID NO: 334 - protein, 15 aa, Artificial Sequence
  CDR3 beta peptide
CSARDPGLEQNIQYF

SEQ ID NO: 335 - protein, 14 aa, Artificial Sequence
  CDR3 beta peptide
CASSSLDGRLGYTF

SEQ ID NO: 336 - protein, 12 aa, Artificial Sequence
  CDR3 beta peptide
CATFTGNTEAFF

SEQ ID NO: 337 - protein, 17 aa, Artificial Sequence
  CDR3 beta peptide
CASSPDDRESSYNEQFF

SEQ ID NO: 338 - protein, 15 aa, Artificial Sequence
  CDR3 beta peptide
CASIRLAGSPYEQYF

SEQ ID NO: 339 - protein, 16 aa, Artificial Sequence
  CDR3 beta peptide
CASSSGLAGRWATQYF

SEQ ID NO: 340 - protein, 7 aa, Artificial Sequence
  CDR3 beta peptide
CATEAFF

SEQ ID NO: 341 - protein, 14 aa, Artificial Sequence
  CDR3 beta peptide
CASSPDGGNTEAFF

SEQ ID NO: 342 - protein, 13 aa, Artificial Sequence
  CDR3 beta peptide
CASSLVLNYEQYF

SEQ ID NO: 343 - protein, 15 aa, Artificial Sequence
  CDR3 beta peptide
CASSLRSGGEETQYF

SEQ ID NO: 344 - protein, 17 aa, Artificial Sequence
  CDR3 beta peptide
CASSPDDRESSYNEQFF

SEQ ID NO: 345 - protein, 14 aa, Artificial Sequence
  CDR3 beta peptide
CASSLEGWDLPLHF

SEQ ID NO: 346 - protein, 12 aa, Artificial Sequence
  CDR3 beta peptide
CASSSAHYGYTF

SEQ ID NO: 347 - protein, 15 aa, Artificial Sequence
  CDR3 beta peptide
CASSLETEKSYEQYF

SEQ ID NO: 348 - protein, 9 aa, Artificial Sequence
  neoantigen peptide
RLFPYALHK

SEQ ID NO: 349 - protein, 10 aa, Artificial Sequence
  neoantigen peptide
ALLPPPPLAK

SEQ ID NO: 350 - protein, 10 aa, Artificial Sequence
  neoantigen peptide
KIYTGEKPYK

SEQ ID NO: 351 - protein, 10 aa, Artificial Sequence
  neoantigen peptide
LLFKAGEMRK

SEQ ID NO: 352 - protein, 10 aa, Artificial Sequence
  neoantigen peptide
RLFSALNSHK

SEQ ID NO: 353 - protein, 9 aa, Artificial Sequence
  M. tuberculosis peptide
GILTVSVAV

SEQ ID NO: 354 - protein, 10 aa, Artificial Sequence
  YML peptide
YMLDLQPETT

SEQ ID NO: 355 - protein, 11 aa, Artificial Sequence
  YML peptide
YMLDLQPETTD

SEQ ID NO: 356 - protein, 13 aa, Artificial Sequence
  YML peptide
YMLDLQPETTDLY

SEQ ID NO: 357 - protein, 9 aa, Artificial Sequence
  SARC-CoV-2 peptide
NLVPMVATV

SEQ ID NO: 358 - protein, 11 aa, Artificial Sequence
  SARS-CoV-2 peptide
NSPRRARSVAS

(19) United States
(12) Patent Application Publication
(10) Pub. No.: US 2024/0239869 A1
(43) Pub. Date: Jul. 18, 2024

(54) SINGLE CHAIN TRIMER MHC CLASS I NUCLEIC ACIDS AND PROTEINS AND METHODS OF USE

(71) Applicants: Institute for Systems Biology, Seattle, WA (US); California Institute of Technology, Pasadena, CA (US)

(72) Inventors: William Chour, San Gabriel, CA (US); James R. Heath, Seattle, WA (US); Jingyi Xie, Seattle, WA (US)

(73) Assignees: Institute for Systems Biology, Seattle, WA (US); California Institute of Technology, Pasadena, CA (US)

(21) Appl. No.: 18/289,674

(22) PCT Filed: May 6, 2022
(86) PCT No.: PCT/US2022/028144

§ 371 Date: Nov. 6, 2023

Related U.S. Application Data
(60) Provisional application No. 63/185,942, filed on May 7, 2021.

(51) Int. Cl. C07K 14/74; A61K 39/00; C40B 40/10

(52) U.S. Cl. CPC C07K 14/70539; A61K 39/4611; A61K 39/4632; C40B 40/10; C07K 2319/01

(57) ABSTRACT

Peptide-major histocompatibility (MHC) Class I nucleic acids and proteins are provided. Methods of their use, for example in methods of identifying antigen-specific T cells and adoptive cell therapy, are also provided.

### CROSS REFERENCE TO RELATED APPLICATION

This application claims the benefit of U.S. Provisional Application No. 63/185,942 filed May 7, 2021, which is incorporated by reference herein in its entirety.

### ACKNOWLEDGMENT OF GOVERNMENT SUPPORT

This invention was made with government support under Contract No. HHSO10020160031C awarded by Biomedical Advanced Research and Development Authority, an agency of the United States Department of Health and Human Services. The government has certain rights in the invention.

### FIELD

This disclosure relates to peptide-major histocompatibility (MHC) Class I nucleic acids and proteins, and methods of their use, for example in methods of adoptive cell therapy.

### BACKGROUND

The emergence of novel, pathogenic virus strains (and the predicted acceleration of such events) has driven the need for high-throughput approaches to epitope-based reagent production. In particular, the use of peptide-MHC (pMHC) reagents to capture antigen-specific T cells can enable identification of relevant T cell receptor (TCR) sequences and shed light on the role played by immunodominant epitopes in the host immune response. Toward this end, vaccine therapies must involve assessment of human leukocyte antigen (HLA) haplotypes and HLA-based epitope landscapes to predict and identify the most prominent immunogenic viral peptides. The number of compatible epitopes per HLA allele may differ vastly, ranging from only a handful up to hundreds or thousands based on the desired scope of inclusion, the natural receptivity of each HLA allele's binding pocket to peptide motifs, and the accuracy of existing peptide binding prediction algorithms. To accommodate this scale, soluble pMHC reagents must be produced on a per-peptide, per-HLA basis in a high-throughput manner to identify and rank immuno-responsive TCRs from peripheral blood mononuclear cells (PBMCs). Soluble pMHCs are conventionally produced by individual expression of the subunits of the MHC within E. coli, followed by subsequent in vitro refolding of the HLA heavy chain and β2-microglobulin (β2m) subunit inclusion bodies in the presence of a target peptide. A modified version to produce the refolded pMHC complex makes use of a UV-cleavable peptide during the reaction. This peptide serves as a placeholder, enabling rapid production of UV-exchanged pMHCs (UV-pMHCs) where UV light exposure facilitates exchange of the cleavable peptide for target peptide. However, the production of refolded pMHCs and UV-pMHCs is prone to several technical problems. Overall protein yield from refolding is HLA-dependent, and the success of UV exchange is highly dependent upon chemico-physical properties of the individual peptide.

Single-chain trimers (SCTs) are an alternative approach to construct pMHCs that may address the issues posed by refolding and UV exchange. Briefly, the SCT format consists of a construct including a peptide, β2m, and HLA. These three primary units, joined to give a single chain, are secreted as a single protein unit. Initially expressed in bacterial cells, SCTs have been adopted into mammalian expression systems.

### SUMMARY

Provided herein are MHC Class I SCTs and assays that can be used for rapid discovery of multiple TCRs from multiple peptides, such as high-throughput assays.

In some embodiments, this disclosure provides nucleic acid fragment pairs including a first nucleic acid fragment and second nucleic acid fragment that, when assembled, encode a major histocompatibility complex (MHC) Class I single chain trimer (SCT) protein, the SCT including as operably linked subunits a peptide, a β2 microglobulin (β2m) protein, and a human leukocyte antigen (HLA) heavy chain protein, and wherein the first nucleic acid fragment and the second nucleic acid fragment each comprise a portion of an assembly site in the β2 microglobulin protein. In some examples, the assembly site is a Gibson assembly site.

In some embodiments, the nucleic acid fragment, when assembled, encodes protein subunits in the following order (N-terminal to C-terminal): a secretion signal, a peptide, a peptide-β2m linker (L1), β2m, a β2m-HLA linker (L2), HLA heavy chain, and optionally, one or more purification tags, and wherein the assembly site is positioned within an invariant region of β2m. In some examples, the secretion signal is selected from an HLA secretion signal, an interferon-α2 secretion signal, and an interferon-γ secretion signal.

In some examples, the nucleic acid fragment pair also encodes one or more purification tags. In particular examples, the one or more purification tags are selected from a peptide that can be biotinylated (e.g., SEQ ID NO: 136) and a polyhistidine peptide.

In some examples, the nucleic acid fragment pair encodes a HLA protein comprising one or more amino acid substitutions selected from the group consisting of H74L, D74L, Y84C, Y84A, A139C, D227K, T228A, and A245V (numbering corresponding to SEQ ID NO: 3).

In some embodiments, the peptide encoded by the nucleic acid fragment pair is an antigen peptide, a self peptide, or a placeholder peptide (e.g., SEQ ID NO: 135). The antigen peptide may be selected from a tumor-associated peptide, a neoantigen peptide, an autoimmune peptide, a fungal peptide, a bacterial peptide, and a viral peptide.

In some embodiments, the nucleic acid fragment pair is codon-optimized for mammalian expression, such as for expression in human cells.

Also provided are nucleic acid molecules that include a disclosed assembled nucleic acid fragment pair. The assembled nucleic acid fragment pair includes the first nucleic acid fragment operably linked to the second nucleic acid fragment. In additional embodiments, the assembled nucleic acid is included in a vector, such as a mammalian expression vector. In one example, the mammalian expression vector is plasmid pcDNA3.1.

Disclosed herein are human cell lines that are transformed with a vector including an assembled nucleic acid molecule described herein. In one example, the human cell line is an HEK293 cell line, such as Expi293F™ cells.

Also provided are libraries that include a plurality of the disclosed nucleic acid fragment pairs or a plurality of the assembled nucleic acid fragment pairs.

Disclosed herein are human-glycosylated MHC Class I SCT proteins. In some examples, the human-glycosylated MHC Class I SCT protein is soluble.

In some embodiments the human-glycosylated MHC Class I SCT protein includes a peptide, such as an antigen peptide, a self peptide, or a placeholder peptide. In one example, the placeholder peptide includes the amino acid sequence of SEQ ID NO: 135. The antigen peptide may be selected from a tumor-associated peptide, a neoantigen peptide, an autoimmune peptide, a fungal peptide, a bacterial peptide, and a viral peptide.

In some embodiments, the soluble human-glycosylated MHC Class I SCT protein includes a peptide, a peptide-β2 microglobulin (β2m) protein linker (L1), a β2m protein, a β2m-HLA linker (L2), and an HLA heavy chain protein, in N-terminal to C-terminal order. In some examples, the human-glycosylated MHC Class I SCT protein includes an HLA protein including one or more amino acid substitutions selected from the group consisting of H74L, D74L, Y84C, Y84A, A139C, D227K, T228A, and A245V. In other examples, the soluble human-glycosylated MHC Class I SCT protein also includes one or more purification tags. In particular examples, the purification tag is a peptide that can be biotinylated (e.g., SEQ ID NO: 136). In other examples, the purification tag is a polyhistidine peptide.

In some embodiments, the soluble human-glycosylated MHC Class I SCT protein is assembled as a stable multimer, such as a stable tetramer. In additional embodiments, the soluble human-glycosylated MHC Class I SCT protein is attached to a surface, a polymer (such as a bead), or a nanoparticle scaffold

Also provided are libraries including a plurality of soluble human-glycosylated MHC Class I SCT proteins or libraries including a plurality of stable multimers of soluble human-glycosylated MHC Class I SCT proteins.

Further disclosed are methods of identifying an antigen-specific CD8+ T cell. In some embodiments, the methods include contacting a T cell population with one or more of the disclosed soluble human glycosylated MHC Class I SCT proteins (such as one or more stable multimers of a soluble human-glycosylated MHC Class I SCT protein) and identifying a CD8+ T cell reactive thereto. In some examples, the methods further include determining the identity of the identified antigen-specific T cell receptor (TCR), for example, by sequencing the TCR, and producing a population of T cells (e.g., CD8+ T cells) expressing the identified TCR.

In some embodiments, the methods also include administering the population of T cells expressing the antigen-specific TCR to a subject in need thereof. In some examples, the subject has cancer (such as a tumor), and the TCR is reactive to an antigen from a tumor sample obtained from the subject.

The foregoing and other features of the disclosure will become more apparent from the following detailed description, which proceeds with reference to the accompanying figures.

### BRIEF DESCRIPTION OF THE DRAWINGS

FIGS. 1A-1C illustrate SCT design for Class I pMHC constructs FIG. 1A shows SCTs encoding Class I pMHC molecules constructed by Gibson assembly from two fragments, enabling modular insertion of any desired Class I HLA subunit to design a template plasmid for peptide insertion. FIG. 1B illustrates template SCT constructs are ligated into pcDNA3.1 vector by restriction digest and ligation. FIG. 1C shows a SCT library containing various peptide elements can be constructed from an initial template plasmid by inverse PCR and ligation.

FIGS. 2A-2C show SCT design and testing. FIG. 2A is an axial view of crystal structure of HLA-A*02:01 SCT (RDB ID: 6APN). Highlighted regions of interest: H74, Y84, A139, first three amino acids of L1 linker. Peptide is loaded into pocket in N-to-C direction (left-to-right). FIG. 2B is a summary of L1 GS moiety (GGGGS; SEQ ID NO: 141; GCGGS, SEQ ID NO: 142; GGCGS, SEQ ID NO: 143; or GCGAS, SEQ ID NO: 144) and HLA amino acid modifications for each of the nine SCT templates tested. Heatmap: Relative expression of each SCT combination, as designated by template (row) and peptide (column). Relative expression is quantified by automated measurement of protein band intensities, as exemplified by reduced SDS-PAGE image of 18 SCTs constructed using design template D9 (bottom). Peptides correspond to SEQ ID NOs: 6-20, 22, 21, and 2 (left to right). Previously expressed and purified aliquot of WT1 (RMFPNAPYL; SEQ ID NO: 1) SCT was used as positive control (+) for band intensity quantification. FIG. 2C shows thermal shift assay measurements of SCTs. Tₘ measurements of two peptides designed using the nine SCT templates are depicted (left). Their Tₘ values are plotted in the scatterplot (right) to show relative changes in stability based on template and peptide. Peptides correspond to SEQ ID NOs: 6-20, 22, 21, and 2 (left to right). Individual thermal shift curves (left) are representative of a biological triplicate measurement, with all individual Tₘs plotted (right).

FIGS. 3A and 3B illustrate that SCT transfection efficiency is uniform and expression is peptide-dependent. FIG. 3A is a graph of Expi293 cells transfected with an SCT library consisting of 15 different peptide elements (x-axis) with or without an IRES-GFP indicator measured for viability and GFP fluorescence after 4 days of transfection. FIG. 3B is a graph showing measurement of SCT protein band intensity in SDS-PAGE performed after transfection using the same plasmid library elements. A negative control (“empty”) consists of Expi293 cells transfected with all standardized reagents except SCT plasmid. For both panels, peptides correspond to SEQ ID NOs: 6, 8, 9, 11, 12, 13, 14, 15, 16, 17, 18, 19, 20, 22, and 21 (left to right).

FIG. 4 shows a flow cytometry assay to optimize WT1 SCT-TCR capture. WT1 (RMFPNAPYL; SEQ ID NO: 1) SCTs constructed according to each of six template designs shown in FIG. 2B were paired with a MART-1 (ELAGIGILTV; SEQ ID NO: 2) SCT (D3 template) to identify their cognate TCR-transduced cells in a 95/5 mixture of C4 TCR-transduced primary T cells and MART-1 Jurkat T cells. Number at top right of each plot indicates the SCT template used for WT1 SCT in the assay. Percentages indicate the proportion of total cell population captured in the WT1 SCT-positive quadrant by each of the six WT1 SCT designs.

FIG. 5 is a series of SDS-PAGE gels showing SCT expression for each of the indicated peptide elements (numbering as in Tables 2 and 3).

FIG. 6 shows functional comparison of CMV pMHC reagents. Left, flow cytometry assays of tetramers prepared using SCT or refolded format. Right, pie charts depicting the unique clonotypes identified by 10× single-cell sequencing of tetramer-positive cells. CDR3α/β sequences are shown in Table 4 and are in the order starting with the largest fraction and proceeds counterclockwise. The offset wedge in the pie charts corresponds to a published pair of CMV-specific CDR3α and CDR3β chains indicating an exact match (LD=0). NLVPMVATV: SEQ ID NO: 44.

FIG. 7 shows ELISpot assay of IFN-γ secreting CD8+ T cells from PBMCs of COVID-19 participants and healthy donors stimulated with peptide pools derived from SARS-COV-2 structural proteins.

FIGS. 8A-8D show expression of SCTs for A*02:01 SARS-COV-2 spike protein epitopes. FIG. 8A is a schematic of the spike protein domains. S, signal sequence; NTD, N-terminal domain; RBD, receptor binding domain; FP, fusion peptide; HR1, heptad repeat 1; CH, central helix; CD, connector domain; HR2, 5 heptad repeat 2; TM, transmembrane domain; CT, cytoplasmic tail; subunits denoted by S1 and S2. Shaded boxes denote relative position and expression yields of SCT proteins. Peptide ID numbers are indexed in descending order of predicted binding affinity. FIG. 8B shows reduced SDS-PAGE of a subset of spike epitope SCTs from FIG. 8A. Lane number indicates peptide ID, with domain-matched background color. +, purified WT1 SCT. FIG. 8C shows bar plots comparing relative SCT yield (quantified against WT1 SCT lanes) and predicted affinity for each peptide from the subset in FIG. 8B. FIG. 8D shows a crystal structure of spike monomer. Domain colors match those of the regions in FIG. 8A; S1 and S2 subunit backbones in white. Amino acids containing the 30 A*02:01 tested epitopes of FIG. 8A in red.

FIGS. 9A-(C shows spike protein-specific T cell populations from COVID-19 participants via NP-NACS. Peptides are plotted according to position on the spike protein, with dashed lines pointing to position along the domain map. In each plot, counts are from two COVID-19 participants and one HLA-matched donor sample (top: A*02:01, middle: B*07:02, bottom: A*24:02). FIG. 9A, SEQ ID NOs: 145-174; FIG. 9B, SEQ ID NOs: 175-196; FIG. 9C SEQ ID NOs: 197-232.

FIGS. 10A and 10B illustrate that SARS-COV-2 spike epitopes induce cytokine secretion in HLA-matched PBMCs. Peptides identified to be immunogenic from the NP-NACS assay were synthesized and used to stimulate HLA-matched PBMCs from InCoV participants and healthy donors for HLA-A*02:01 (FIG. 10A) and HLA-B*07:02 (FIG. 10B). KLPDDFTGCV (SEQ ID NO: 114), RLDKVEAEV (SEQ ID NO: 113), SIIAYTMSL (SEQ ID NO: 188), MIAQYTSAL (SEQ ID NO: 192).

FIG. 11 is a plot of PLpro-specific T cell populations from A*02:01COVID-19 participants via NP-NACS. Peptides are plotted along x-axis according to relative position on nsp3 protein and color-coded by nsp3 subunit (UBL: ubiquitin-like domain, Ac: Glu-rich acidic-domain, ADRP: ADP-ribose-1′-phosphatase domain, SUD: SARS unique domain, PLpro: papain-like protease, NAB: nucleic acid binding domain, G2M: marker domain, TM: transmembrane domain, ZF: zinc finger domain, Y1-Y2-Y3: Y domains preceding PLpro cleavage site). Peptides are SEQ ID NOs: 233-307 (left to right).

FIGS. 12-12C show frequencies of antigen-specific T cell populations identified by individual tetramer sorting from expanded T cells for COVID-19 participants (y-axis) of three HLA alleles (top, A*02:01; middle, B*07:02; bottom, A*24:02). FIG. 12A, SEQ ID NOs: 146-149, 151-152, 155-166, 168, 170-174, and 357; FIG. 12B, SEQ ID NOs: 175-188, 190-196, and 358; FIG. 12C, SEQ ID NOs: 197-232.

FIG. 13 shows frequencies of antigen-specific T cell populations among the top 20 most common detected clonotypes, identified by multiplexed dextramer sorting from expanded T cells for COVID-19 participants. “Dextramer” refers to the ID of the dextramer shown in Table 5. CDR3α sequences are SEQ ID NOs: 308-327 (left to right) and CDR3β sequences are SEQ ID NOs: 328-347 (left to right).

FIG. 14 shows that transduced TCRs are specific to SARS-COV-2 antigens. TCRs obtained by 10× or bulk sequencing methods from healthy donor or COVID-19 participant-derived T cells were transduced into HLA-matched CD8+ T cells and selectively expanded after SCT tetramer binding to generate cell lines. Shown here are the tetramer binding results of the expanded cells, demonstrating SCT specificity and purity of the cell lines.

FIG. 15 shows T cells transduced with TCRs 001 & 002 corresponding to peptides 1 and 2, respectively, that were functionally assessed after 16-hour overnight peptide stimulation. Top: ELISA assay measuring cytokine release. Middle: ELISpot assay counting cells with granzyme B expression. Bottom: Flow cytometry assay measuring percentage of cells activated (CD137+) and cytotoxic (granzyme B+). Peptide 1: SEQ ID NO: 131; peptide 2: SEQ ID NO: 121.

FIGS. 16A and 16B demonstrate that D227K and T228A mutations inhibit CD8 interaction with pMHCs. FIG. 16A is SDS-PAGE of A*02:01 SCTs expressed with the WT1 epitope (RMFPNAPYL; SEQ ID NO: 1) for various templates. Labels above each bracket indicate the CD8-inhibiting mutation applied to each set of SCTs (“wild-type” refers to no mutation against CD8 interaction). +, purified WT1 SCT. Lane 8's cells were found to be low viability, so no transfection occurred, leading to no detectable SCT output for this plasmid. FIG. 16B. is flow cytometry intensity plots of tetramer binding interaction between expressed WT1 SCTs and TCR-transduced T cells. Y-axis denotes SCT type (colors correspond with legend in FIG. 16A). Binding experiments were performed with CD8+ T cells (left column) and CD4+ T cells (right column). In each plot, the dashed line indicates the positive signal threshold of 10³ mean fluorescence intensity units (right of line=positive).

FIGS. 17A and 17B show that A245V mutation inhibits CD8 interaction with pMHCs loaded with neoantigens. FIG. 17A is flow cytometry profiles of neoantigen-loaded A*03:01 SCT tetramers incubated with PBMCs from a melanoma patient. Lower left quadrant indicates non-binding. FIG. 17B shows the experiment in FIG. 17A, expanded to cover various other combinations of SCT tetramers. Lower left quadrant indicates non-binding. SLHAHGLSYK (SEQ ID NO: 134); RLFPYALHK (SEQ ID NO: 348); ALLPPPPLAK (SEQ ID NO: 349); KIYTGEKPYK (SEQ ID NO: 350); LLFKAGEMRK (SEQ ID NO: 351); RLFSALNSHK (SEQ ID NO: 352).

FIG. 18 shows flow cytometry of PBMCs from an A*02:01-positive healthy donor incubated with SCT tetramers encoding positive control peptides (from EBV, CMV, and influenza) and negative control peptide (from M. tuberculosis). YVLDHLIVV (SEQ ID NO: 27); NLVPMVATV (SEQ ID NO: 44); FMYSDFHFI (SEQ ID NO: 45); GILTVSVAV (SEQ ID NO: 353).

FIG. 19 shows SDS-PAGE analysis of transfected SCT plasmids modified with combinations of various peptide lengths (8-14mer: from YMLDLQPE (SEQ ID NO: 4) to YMLDLQPETTDLYC (SEQ ID NO: 5)) and various template designs. +, purified WT1 SCT. L1 GS moieties: GGGGS, SEQ ID NO: 141; GCGGS, SEQ ID NO: 142; GGCGS, SEQ ID NO: 143; or GCGAS, SEQ ID NO: 144.

FIG. 20 is a scatter plot of Tm values of YML SCTs, color-coded by design template and arranged left-to-right by peptide length. Biological triplicate measurements were performed for each peptide/template SCT combination. One plasmid failed to express during transfection due to human error (D4 SCT loaded with 10mer), so no measurements could be performed for that sample. YMLDLQPE (SEQ ID NO: 4); YMLDLQPET (SEQ ID NO: 6); YMLDLQPETT (SEQ ID NO: 354); YMLDLQPETTD (SEQ ID NO: 355); YMLDLQPETTDL (SEQ ID NO: 7); YMLDLQPETTDLY (SEQ ID NO: 356); YMLDLQPETTDLYC (SEQ ID NO: 5).

FIG. 21 is a schematic illustration of an exemplary embodiment of adoptive cell therapy (ACT). This immunotherapy method begins with extraction of tissue (1) to identify antigens (2), such as neoantigens, if the subject has a tumor. Peptide-MHC binding affinity predictions are performed (3) to identity the best peptide candidates for pMHC generation (4). Stable pMHCs are then tetramerized and used to capture antigen-specific T cells (5), whose TCRs are subsequently sequenced (6), synthesized in plasmid constructs (7), transformed into healthy T cells (8), and administered to the subject (9). Alternatively, the subject could be vaccinated with the peptide candidates (non-ACT route).

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.
SEQ ID NO: 1 is a Wilm's tumor 1 (WT1) peptide.
SEQ ID NO: 2 is a MART-1 peptide.
SEQ ID NO: 3 is the amino acid sequence of the extracellular domain of an exemplary HLA protein (A*02:01) amino acid sequence (lacking signal sequence, transmembrane domain, and intracellular portion). Underlined residues are positions of exemplary amino acid substitutions discussed herein:

GSHSMRYFFTSVSRPGRGEPRFIAVGYVDDTQFVRFDSDAASQRMEPRAPWIEQEGPEYWDGETRK
VKAHSQTHRVDLGTLRGYYNQSEAGSHTVQRMYGCDVGSDWRFLRGYHQYAYDGKDYIALKEDLRS
WTAADMAAQTTKHKWEAAHVAEQLRAYLEGTCVEWLRRYLENGKETLQRTDAPKTHMTHHAVSDHE
ATLRCWALSFYPAEITLTWQRDGEDQTQDTELVETRPAGDGTFQKWAAVVVPSGQEQRYTCHV
QHEGLPKPLTLRWEPSSQPT

SEQ ID NOs: 4 and 5 are HPV E7 peptides.
SEQ ID NOs: 6-21 are additional peptides used for SCT library template optimization studies.
SEQ ID NO: 22 is an additional WT1 peptide.
SEQ ID NOs: 23-58 are A*02:01 viral antigens.
SEQ ID NOs: 59-88 are A*24:02 viral antigens.
SEQ ID NOs: 89-100 are TCR CDR3α sequences.
SEQ ID NOs: 101-112 are TCR CDR3β sequences.
SEQ ID NOs: 113-133 are CoV-2 peptides.
SEQ ID NO: 134 is an additional antigen peptide.
SEQ ID NO: 135 is an exemplary placeholder peptide: SALSEGATPQDLNTML
SEQ ID NO: 136 is the amino acid sequence of a purification tag that can be biotinylated by biotin ligase: GLNDIFEAQKIEWHE
SEQ ID NOs: 137-144 are exemplary glycine-serine peptide linker sequences or GS moieties:

(SEQ ID NO: 137)
GGGGSGGGGSGGGGS
(SEQ ID NO: 138)
GCGGSGGGGSGGGGS
(SEQ ID NO: 139)
GCGASGGGGSGGGGS
(SEQ ID NO: 140)
GGGGSGGGGSGGGGSGGGGS
(SEQ ID NO: 141)
GGGGS
(SEQ ID NO: 142)
GCGGS
(SEQ ID NO: 143)
GGCGS
(SEQ ID NO: 144)
GCGAS

SEQ ID NOs: 145-307 are additional SARS-COV-2 peptides.
SEQ ID NOs: 308-327 are additional CDR3 alpha sequences.
SEQ ID NOs: 328-347 are additional CDR3 beta sequences.
SEQ ID NOs: 348-352 are neoantigen peptides.
SEQ ID NO: 353 is a M. tuberculosis peptide.
SEQ ID NOs: 354-356 are additional YML peptides.
SEQ ID NOs: 357-358 are additional SARS-COV-2 peptides

### DETAILED DESCRIPTION

Provided herein is a high-throughput SCT expression platform enabling production of SCTs for any pairing of peptide and Class I HLA allele. Whereas with traditional pMHC folding, epitope and HLA modularity are determined by peptide synthesis and refolded MHC subunits, respectively, the SCT platform described herein utilizes a primer and a PCR template plasmid to determine these two variables. The facile nature of handling and scaling up these PCR reagents enables a mix-and-match approach that allows rapid screening across a peptide library and list of HLA template variants to optimize pMHCs.

This system was initially applied for a test case of 18 tumor-associated antigens (TAAs) for HLA-A*02:01, utilizing nine different L1/HLA templates, in order to two-dimensionally assess the impact of peptide identity and L1/HLA templates on SCT protein expression and thermal stability. Next, the functionality of these SCTs in a disease context was assessed by assembling HLA-A*02:01 and A*24:02 SCTs loaded with epitopes derived from common viral strains, demonstrating that they can bind to healthy donor T cells stimulated against the synthesized forms of these epitopes.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); George P. Rédei, Encyclopedic Dictionary of Genetics, Genomics, Proteomics and Informatics, 3^{rd} Edition, Springer, 2008 (ISBN: 1402067534); and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:
Autologous: Refers to tissues, cells or nucleic acids taken from an individual's own tissues. For example, in an autologous transfer or transplantation of T cells, the donor and recipient are the same person. Autologous (or “autogeneic” or “autogenous”) is related to self, or originating within an organism itself.
Human leukocyte antigen (HLA): Proteins encoded by the MHC gene complex. HLAS from MHC Class I include HLA-A, HLA-B, and HLA-C genes and are highly variable, with up to hundreds of variant alleles at some loci. HLA loci are named with HLA, followed by the locus (e.g., A), and a number (such as 01:01) designating a specific allele at the locus (e.g., HLA-A*01:01 or HLA-B*07:02).
Linker: A nucleic acid or amino acid sequence that connects (e.g., covalently links) two nucleic acid or amino acid segments. In some examples, linker sequences may be included to provide rotational freedom to linked polypeptide domains and thereby to promote proper domain folding and inter- and intra-domain bonding. Linkers may be native sequences (for example, those found in naturally occurring MHC Class I proteins) or may be recombinant or artificial sequences. In one non-limiting example, linker sequences include glycine-serine amino acid sequences (or a nucleic acid sequence encoding the amino acid sequence), which include varying numbers of glycine and serine residues (e.g., glycine(4)-serine).
Major histocompatibility complex (MHC) Class I: MHC class I molecules are heterodimers formed from two non-covalently associated proteins, the HLA heavy chain (also referred to as HLA α chain herein) and β2-microglobulin. The HLA heavy chain includes three distinct domains, α1, α2 and α3. The three-dimensional structure of the α1 and α2 domains forms the groove into which antigen fit for presentation to T-cells. The α3 domain is an Ig-fold like domain that contains a transmembrane sequence that anchors the α chain into the cell membrane of the APC. MHC class I complexes, when associated with antigen (and in the presence of appropriate co-stimulatory signals) stimulate CD8 cytotoxic T-cells, which function to kill any cell which they specifically recognize.
Nucleic acid fragment: A nucleic acid sequence (such as a linear sequence) of any length that, when assembled with (e.g., operably linked to) at least one other nucleic acid fragment, produces a complete nucleic acid molecule. In some embodiments, assembly of at least two nucleic acid fragments produces a nucleic acid that encodes an MHC Class I SCT of the disclosure.
Operably linked: A first nucleic acid is operably linked with a second nucleic acid when the first nucleic acid is placed in a functional relationship with the second nucleic acid. For instance, a promoter is operably linked to a coding sequence if the promoter affects the transcription or expression of the coding sequence. Where necessary to join two protein coding regions, the open reading frames are aligned. Similarly, proteins (including protein subunits, domains, and/or peptides) are operably linked when they are placed in a functional relationship with one another. In some examples, the operably linked segments are in an arrangement that does not occur in nature. Linkers may be included between nucleic acid or protein segments.
Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.
Single chain trimer (SCT): A recombinant MHC Class I molecule including all portions of the complex (HLA heavy chain, β2m, and peptide) as a single, linked molecule. In some examples, SCT refers to a nucleic acid encoding an HLA heavy chain, β2m, peptide antigen, and one or more linkers. In other examples, SCT refers to the protein.
Subject: A living multi-cellular vertebrate organism, a category that includes both human and veterinary subjects, including human and non-human mammals.
T cell: A white blood cell (lymphocyte) that is an important mediator of the immune response. T cells include, but are not limited to, CD4⁺ T cells and CD8⁺ T cells. A CD4⁺ T cell is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8⁺ T cells can also be detected by an increase in cytolytic activity in response to an antigen.
T cell receptor (TCR): A heterodimeric protein on the surface of a T cell that binds an antigen (such as an antigen bound to an MHC molecule, for example, on an antigen presenting cell). TCRs include α and β chains, each of which is a transmembrane glycoprotein. Each chain has variable and constant regions with homology to immunoglobulin variable and constant domains, a hinge region, a transmembrane domain, and a cytoplasmic tail. Similar to immunoglobulins, TCR gene segments rearrange during development to produce complete variable domains.

T cells are activated by simultaneous binding of their TCRs and co-stimulatory molecules to peptide-bound major histocompatibility complexes and complementary co-stimulatory molecules on antigen-presenting cells, respectively. For example, a CD8⁺ T cell bears T cell receptors that recognize a specific epitope when presented by a particular HLA molecule on a cell. When a CTL precursor that has been stimulated by an antigen presenting cell to become a cytotoxic T lymphocyte contacts a cell that bears such an HLA-peptide complex, the CTL forms a conjugate with the cell and destroys it.
Transduced and Transformed: A vector “transduces” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule is introduced into a cell, including transformation with plasmid vectors, and introduction of naked DNA by electroporation, lipofection, and particle gun acceleration.
Treating or inhibiting a condition: “Treating” a condition refers to a therapeutic intervention that ameliorates a sign or symptom of a disease or pathological condition after it has begun to develop. “Inhibiting” refers to inhibiting the full development of the disease or condition. Inhibition of a condition can span the spectrum from partial inhibition to substantially complete inhibition of the condition. In some examples, the term “inhibiting” refers to reducing or delaying the onset or progression of a disease. A subject to be treated can be identified by standard diagnosing techniques for such a disorder, for example, based on signs and symptoms, family history, and/or risk factors to develop the disease or disorder.
Vector: A nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of an inserted gene or genes. In some non-limiting examples, the vector is a mammalian expression vector.

### II. MHC Class I SCT Nucleic Acids and Libraries

Disclosed herein are nucleic acids encoding MHC Class I SCTs and libraries including the nucleic acids. In some embodiments, the nucleic acids are provided as two or more nucleic acid fragments that when assembled encode an MHC Class I SCT. In particular examples, the SCTs are assembled from a pair of nucleic acid fragments; however, more than two nucleic acid fragments (such as 3, 4, or more) could also be utilized, by using multiple assembly sites to generate the final nucleic acid encoding the SCT.

In embodiments, provided are a nucleic acid fragment pair including a first nucleic acid fragment and second nucleic acid fragment that, when assembled, encode a major histocompatibility complex (MHC) Class I single chain trimer (SCT) protein. The SCT encoded by the assembled nucleic acid fragment pair includes as operably linked subunits a peptide (such as a peptide antigen), a β2m protein and an HLA heavy chain. The first nucleic acid fragment and the second nucleic acid fragment each include a portion of an assembly site in a position, that, when the first nucleic acid fragment and the second nucleic acid fragment are assembled, encodes an invariant region in β2m of the encoded MHC Class I SCT protein. In particular examples, the assembly site is a Gibson assembly site (see, e.g., Gibson et al., Nature Methods 6:343-345, 2009). In other examples, the assembly site is a restriction enzyme site.

In some embodiments, the nucleic acid fragment pair further includes a nucleic acid sequence that encodes a purification tag. In some examples, the purification tag is a polyhistidine tag (such as a 6× His tag). In other examples, the purification tag is an amino acid sequence that can be biotinylated by biotin ligase. In one example, the purification tag encodes the amino acid sequence GLNDIFEAQKIEWHE (SEQ ID NO: 136). In some examples, the nucleic acid fragment pair includes nucleic acid sequences that encode two or more purification tags (such as a 6× His tag and a peptide that can be biotinylated).

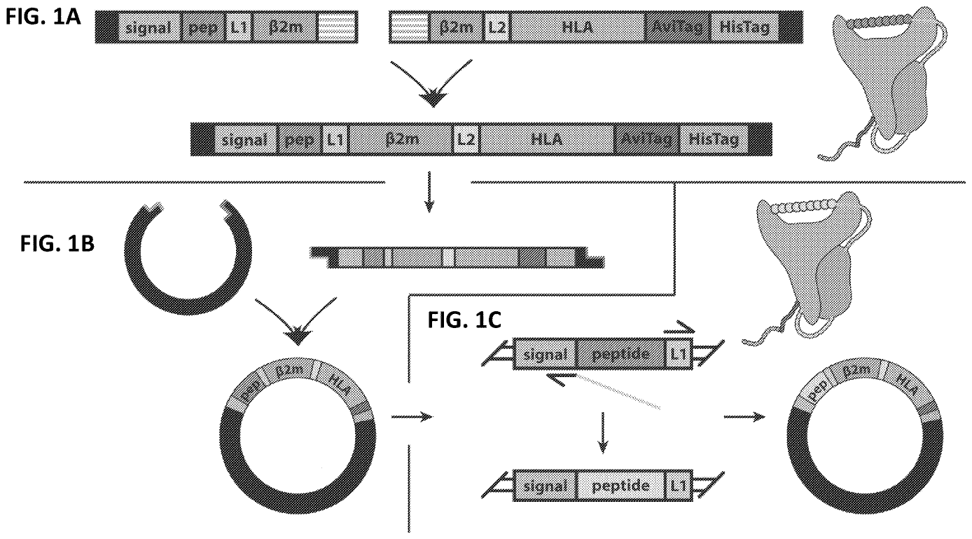

The disclosed nucleic acid fragments (such as nucleic acid fragment pairs) provide for modular combination of different peptides (such as different antigen peptides) with different HLA heavy chains. In some examples, peptide substitution is achieved by a PCR-based method, such as inverse PCR. For example, a reverse primer encoding the reverse complement of a desired peptide is used in combination with a universal forward primer (such as a universal forward primer that binds to a sequence in linker L1). This is illustrated schematically in FIG. 1C. In other examples, overlapping primers that encode a desired peptide are used to assemble a double-stranded construct including restriction enzyme recognition sites at the 5′ and 3′ ends that correspond to restriction enzyme sites flanking the peptide in the SCT template. The double-stranded construct and the SCT template are digested with the restriction enzyme(s) and ligated to produce the full-length construct.

In some embodiments, the assembled nucleic acid fragment pair encodes an SCT with protein subunits in the order (N-terminal to C-terminal): a secretion signal, a peptide (such as a peptide antigen or placeholder peptide), a first linker (L1), a β2m protein, a second linker (L2), and an HLA heavy chain. In some embodiments, the secretion signal is an HLA secretion signal (such as an HLA α secretion signal). However, other secretion signals can be used, including, but not limited to a secretion signal from human β2m, human interferon (IFN)-α2, human IFNγ, human interleukin-2, human serum albumin, human IgG heavy chain, or Gaussia princeps luciferase. If desired, one of ordinary skill in the art can test one or more secretion signals to identify one or more that provide increased or optimized expression levels of an SCT.

In some examples, L1 encodes a glycine-serine linker, such as the amino acid sequence of any one of SEQ ID NOs: 137-139. In some examples, L2 also encodes a glycine-serine linker, for examples SEQ ID NO: 137 or SEQ ID NO: 140. In additional examples, a third linker (L3) may be included between the HLA α chain and a purification tag (if included). In some examples, L3 encodes the amino acid sequence GG.

In some embodiments, the disclosed nucleic acid fragment pairs, when assembled, encode soluble SCTs. In some embodiments, the HLA heavy chain is the extracellular domain of an HLA heavy chain protein. Thus, in some examples, the transmembrane domain and intracellular domain of HLA heavy chain are not included. The HLA α secretion signal may be removed (for example, if the HLA α chain is internal to the SCT). In other embodiments, the disclosed nucleic acid fragment pairs, when assembled, encode membrane bound SCTs. In such embodiments, the nucleic acid fragment pair encodes HLA heavy chain extracellular, transmembrane, and cytoplasmic domains.

In some embodiments, the HLA heavy chain is a human HLA heavy chain or a mouse HLA heavy chain. In some examples, the human HLA heavy chain is selected from an HLA-A, HLA-B, or HLA-C heavy chain. In other examples, the mouse HLA heavy chain is a H-2K, H-2D, or H-2L heavy chain. The amino acid and nucleic acid sequences of HLA heavy chain alleles for each locus are publicly available, for example from EMBL-EBI (e.g., ftp.ebi.ac.uk/pub/databases/ipd/imgt/hla/fasta/). One of ordinary skill in the art can identify other sources or sequence databases, along with updates. In some examples, the HLA heavy chain is included in an HLA heavy chain-encoding fragment library.

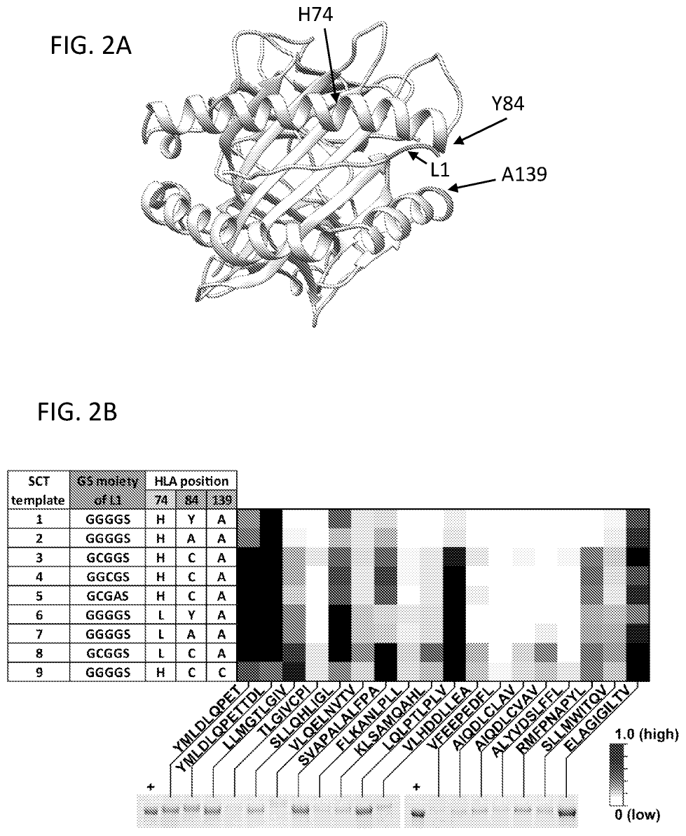

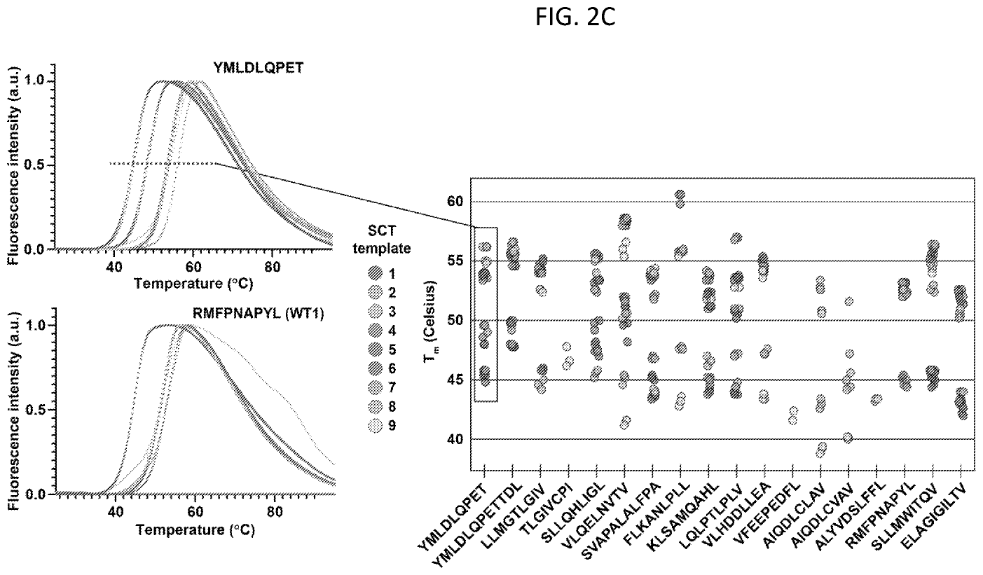

In some embodiments, the HLA heavy chain encoded by the nucleic acid fragments disclosed herein includes one or more amino acid substitutions compared to a wild type HLA heavy chain. Amino acid substitutions may be selected to improve the properties or function of the SCT encoded by the assembled pair of nucleic acid fragments, such as increasing stability, peptide loading in the peptide binding groove, immunogenicity, and/or enabling dithiol linkage. Exemplary amino acid substitutions include a leucine at an amino acid position corresponding to amino acid 74 of SEQ ID NO: 3 (e.g., H74L or D74L), a cysteine or a leucine at an amino acid position corresponding to amino acid 84 of SEQ ID NO: 3 (e.g., Y84C or Y84L), a cysteine at an amino acid position corresponding to amino acid 139 of SEQ ID NO: 3 (e.g., A139C), or any combination of two or more thereof. Exemplary combinations of amino acid substitutions include those illustrated for SCT templates 1-9 in FIG. 2B. In other embodiments, an amino acid substitution that reduces pMHC interaction with the CD8 co-receptor on T cells is included. SCTs with one or more of such amino acid substitutions may be useful to skew successful binding interactions toward TCRs with high affinity for pMHC, e.g., as a filter to remove low-affinity TCRs from an antigen-specific T cell population. In some examples, the amino acid substitution includes a lysine at an amino acid position corresponding to amino acid 227 of SEQ ID NO: 3 (e.g., D227K), an alanine at an amino acid position corresponding to amino acid 228 of SEQ ID NO: 3 (e.g., T228A), a valine at an amino acid position corresponding to amino acid 245 of SEQ ID NO: 3 (e.g., A245V), or any combination of two or more thereof. In some embodiments, the HLA α chain includes one or more of H74L, Y84C, Y84A, A139C, D227K, T228A, and A254V, with the amino acid positions corresponding to those of SEQ ID NO: 3.

In some embodiments, the peptide included in the disclosed SCTs is a peptide antigen, a placeholder peptide, a self peptide (such as a peptide that occurs in healthy tissue, and is not mutated), a negative control peptide, or a positive control peptide. In some embodiments, the placeholder peptide provides “space” for the peptide-encoded region of the reverse primer to overlay (e.g., as shown in FIG. 1C), or to serve as the fragment that is removed during peptide substitution. For peptide substitution by restriction enzyme digestion, the placeholder peptide may provide spacing between enzyme cut sites to prevent or minimize spatial interference between the restriction enzymes during cleavage. Thus, in some examples, the placeholder peptide may be at least four amino acids long. In examples utilizing inverse PCR, a placeholder peptide may not be required, and is optional. Thus, in some examples, a placeholder peptide is from about 4-25 amino acids in length. In other examples, no placeholder peptide is present (that is, the peptide is 0 amino acids in this situation). In one example, a placeholder peptide is HIV GAG amino acids 173-188 and has the amino acid sequence SALSEGATPQDLNTML (SEQ ID NO: 135). However, other placeholder peptide sequences could be utilized, or could even be omitted in some situations, as discussed above.

In some embodiments, the peptide is a peptide antigen. A peptide antigen is a peptide that fits in the binding pocket of an MHC Class I protein complex or an MHC Class I SCT protein and is recognized by CD8⁺ T cells. In some embodiments, the peptide is about 8-14 amino acids long (e.g., 8, 9, 10, 11, 12, 13, 14 amino acids long). However, peptide antigens that are longer or shorter could also be utilized. Typically, a positive control and/or negative control peptide would be the same length as a target peptide (such as a peptide antigen), or about 8-14 amino acids long. In some examples, the peptide antigen is a tumor-associated peptide, a neoantigen peptide, an autoimmune peptide (such as a self peptide that is auto-reactive), a fungal peptide, a bacterial peptide, or a viral peptide (such as an influenza virus peptide, a coronavirus peptide, a human immunodeficiency virus (HIV) peptide, a human papillomavirus (HPV) peptide, a cytomegalovirus (CMV) peptide, a hepatitis virus peptide (e.g., HBV or HCV peptide), an Epstein Barr virus (EBV), or a rotavirus peptide). In some examples, the peptide antigen is selected from any one of SEQ ID NOs: 23-88 and 115-132.

Also provided herein are libraries that include a plurality of the nucleic acid fragment pairs disclosed herein. In some embodiments, the library includes 2 or more nucleic acid fragment pairs, such as 2-500 (for example, 2-50, 10-100, 20-200, 75-150, 200-400, or 300-500) nucleic acid fragment pairs. The library, in some examples, includes nucleic acid fragments encoding a plurality of HLA α chains and a plurality of peptides. Thus, in some examples, the library of nucleic acid fragment pairs can be used for modular construction of nucleic acids encoding a plurality of SCTs disclosed herein.

In some embodiments, the library includes two subsets, wherein a first subset includes a plurality of first nucleic acid fragments of the pair and a second subset includes a plurality of second nucleic acid fragments of the pair. In some examples, the first nucleic acid fragments each include at least a nucleic acid encoding a peptide and a portion of β2m and the second nucleic acid fragments each include at least a nucleic acid encoding a portion of β2m and HLA α chain.

In some embodiments, the nucleic acid sequences encoding one or more of the SCT components of the nucleic acid fragments disclosed herein may be altered by taking advantage of the degeneracy of the genetic code such that, while the nucleotide sequence is altered, it nevertheless encodes a peptide having an amino acid sequence identical to the peptide sequences. Based upon the degeneracy of the genetic code, variant DNA molecules may be derived from the nucleic acid sequences disclosed herein or known to one of skill in the art using standard DNA mutagenesis techniques or by synthesis of DNA sequences. Thus, this disclosure also encompasses nucleic acid sequences which encode the subject SCTs, but which vary from the disclosed nucleic acid sequences by virtue of the degeneracy of the genetic code.

The nucleic acid fragments provided herein may further be codon-optimized for expression in mammalian cells. In some embodiments, the nucleic acid fragments are codon-optimized for expression in human cells. A codon-optimized nucleic acid refers to a nucleic acid sequence that has been altered such that the codons are optimal for expression in a particular system (such as a particular species or group of species). Codon optimization does not alter the amino acid sequence of the encoded protein. In some examples, codon-optimization refers to replacement of at least one codon (such as at least 5 codons, at least 10 codons, at least 25 codons, at least 50 codons, at least 75 codons, at least 100 codons or more) in a nucleic acid sequence with a synonymous codon (one that codes for the same amino acid) more frequently used (preferred) in the particular organism of interest (such as humans). Each organism has a particular codon usage bias for each amino acid, which can be determined, for example, from publicly available codon usage tables (for example see Nakamura et al., Nucleic Acids Res. 28:292, 2000). For example, a codon usage database is available on the World Wide Web at kazusa.or.jp/codon. One of skill in the art can modify a nucleic acid encoding a particular amino acid sequence, such that it encodes the same amino acid sequence, while being optimized for expression in a particular cell type (such as a human cell). Additional criteria that can be applied for codon optimization include GC content (such as average overall GC content of about 50% or about 50% GC content over given window length (such as about 30-60 bases)) and avoidance of sequences that must not be included (such as a particular restriction enzyme recognition site). In some examples, a codon-optimized sequence is generated using software, such as codon-optimization tools available from Integrated DNA Technologies (Coralville, IA, available on the World Wide Web at idtdna.com/CodonOpt), GenScript (Piscataway, NJ), or Entelechon (Eurofins Genomics, Ebersberg, Germany, available on the World Wide Web at entelechon.com/2008/10/backtranslation-tool/).

Also provided are nucleic acid molecules assembled from the nucleic acid fragments (such as nucleic acid fragment pairs) disclosed herein. The assembled nucleic acid is prepared using the assembly sites present in the nucleic acid fragments. Thus, in some examples, the nucleic acid molecule is assembled by Gibson assembly. In other examples, the nucleic acid molecule is assembled by restriction enzyme digestion and ligation of the digested fragments. The assembled nucleic acid fragments are operably linked, such that the first nucleic acid fragment and second nucleic acid fragment are contiguous and the protein coding sequences are in frame.

In additional embodiments, a library including a plurality of the assembled nucleic acid molecules is also provided. In some embodiments, the library includes 2 or more such as 2-2500 (for example, 2-25, 5-50, 10-100, 20-200, 75-150, 200-400, 300-500, 400-600, 500-750, 600-800, 700-1000, 1000-1500, 1250-1750, 1500-2000, or 2000-2500) of the assembled nucleic acids. In some examples, the library of assembled nucleic acids encodes a plurality of SCTs that differ in one or more of the encoded HLA α chains and/or peptides. Peptides of interest can be inserted into each combination of HLA α chain and β2m, as desired. In some examples, the library size of HLA α chains is narrowed, for example, using an algorithm to rank peptide-HLA pairs for binding affinity. Alternatively, a single SCT HLA α chain is selected and a library of assembled nucleic acids is prepared, with each member having the same HLA, but a different peptide.

In some embodiments, the nucleic acid molecule assembled from the nucleic acid fragments (such as an assembled nucleic acid fragment pair) is included in a vector. In some examples, the vector further includes one or more expression control sequences operably linked to the assembled nucleic acid, such that expression of the assembled nucleic acid is achieved under conditions compatible with the expression control sequences. The expression control sequences can include, but are not limited to, appropriate promoters, enhancers, transcription terminators, ribosome biding sequence, a start codon (e.g., ATG) 5′ of a protein-encoding nucleic acid, maintenance of the correct reading frame of that gene to permit proper translation of mRNA, and stop codons. The expression control sequence(s) in some examples are heterologous expression control sequence(s), for example from source other than the protein-encoding nucleic acid. Thus, the protein-encoding nucleic acid operably linked to a heterologous expression control sequence (such as a promoter) comprises a nucleic acid that is not naturally occurring. The vector may further include one or more additional elements, such as an origin of replication, one or more selectable marker genes (such as one or more antibiotic resistance genes), or other elements known to one of ordinary skill in the art.

Vectors for cloning, replication, and/or expression of the assembled nucleic acid molecules include bacterial plasmids, such as bacterial cloning or expression plasmids (some of which can be used for expression in bacterial and/or mammalian cells). Exemplary bacterial plasmids into which the nucleic acids can be cloned include E. coli plasmids, such as pBR322, pUC plasmids (such as pUC18 or pUC19), pBluescript, pACYC184, pCD1, pGEM® plasmids (such as pGEM®-3, pGEM®-4, pGEM-T® plasmids; Promega, Madison, WI), TA-cloning vectors, such as pCR® plasmids (for example, pCR® II, pCR® 2.1, or pCR® 4 plasmids; Life Technologies, Grand Island, NY) or pcDNA plasmids (for example pcDNA™3.1 or pcDNA™3.3 plasmids; Life Technologies). In some examples, the vector includes a heterologous promoter which allows protein expression in bacteria. Exemplary vectors include pET vectors (for example, pET-21b), pDEST™ vectors (Life Technologies), pRSET vectors (Life Technologies), pBAD vectors, and pQE vectors (Qiagen).

In other embodiments, the vector is a mammalian expression vector. In some examples, mammalian expression vectors include a constitutive promoter, such as a CMV promoter. In other examples, the vector includes a viral origin of replication (such as an Epstein-Barr virus or SV40 origin of replication) that permits replication of the plasmid in a transformed mammalian cell. In one non-limiting example, the mammalian expression vector is a pcDNA™3 vector, for example, pcDNA™3.1 vector (ThermoFisher Scientific). However, it should be recognized that many mammalian expression vectors are available, and suitable alternatives can be selected by one of ordinary skill in the art.

Also provided are host cells, such as mammalian cells, that are transformed with a vector including an assembled nucleic acid molecule encoding an MHC Class I SCT. As utilized herein, the term “host cell” also includes any progeny of the subject host cell. Methods of transient expression or stable transfer, meaning that the foreign DNA is continuously maintained in the host, are known in the art. Techniques for the propagation of mammalian cells in culture are known to one of ordinary skill in the art. Examples of commonly used mammalian host cell lines are HEK293 cells, VERO cells, HeLa cells, CHO cells, WI38 cells, BHK cells, and COS cell lines, although other cell lines may be used, such as cells designed to provide improved expression, desirable glycosylation patterns, or other features. In some non-limiting examples, the mammalian host cells are HEK293 cells, such as Expi293F™ cells (ThermoFisher Scientific).

Transformation of a host cell with recombinant DNA can be carried out by techniques known to those skilled in the art. When the host is a eukaryote, methods including transfection of DNA as calcium phosphate coprecipitates, mechanical procedures such as microinjection, electroporation, insertion of a plasmid encased in liposomes, or viral vectors can be used.

### III. Human SCT Proteins

Disclosed herein are human MHC Class I single chain trimer proteins, such as those encoded by the nucleic acid fragment pairs and assembled nucleic acids described above. As discussed in Section II, in some embodiments, mammalian host cells transformed with nucleic acid(s) encoding the disclosed SCTs are provided. In some embodiments, the human MHC Class I SCTs are soluble. In addition, as a result of expression in mammalian cells (for example, in contrast to bacterial or insect cells), the SCTs may include post-translational modifications representative of pMHCs expressed in human cells and/or are properly folded and generate functional proteins, for example at higher efficiency than those produced in non-mammalian systems. In particular embodiments, the SCTs are glycosylated.

Any of the SCTs encoded by the nucleic acid fragment pairs or assembled nucleic acids described in Section II can be produced as soluble human glycosylated MHC Class I SCTs. Thus, in some embodiments, the soluble human glycosylated MHC Class I SCT has the organization of: a secretion signal, a peptide (such as a peptide antigen or placeholder peptide), a first linker (L1), a β2m protein, a second linker (L2), and an HLA heavy chain, in N-terminal to C-terminal order. The SCT may also include a purification tag.

In some embodiments, the soluble human glycosylated MHC Class I SCT includes one or more amino acid substitutions compared to a wild type HLA heavy chain. Exemplary amino acid substitutions include a leucine at an amino acid position corresponding to amino acid 74 of SEQ ID NO: 3 (e.g., H74L or D74L), a cysteine or a leucine at an amino acid position corresponding to amino acid 84 of SEQ ID NO: 3 (e.g., Y84C or Y84L), a cysteine at an amino acid position corresponding to amino acid 139 of SEQ ID NO: 3 (e.g., A139C), or any combination of two or more thereof. Exemplary combinations of amino acid substitutions include those illustrated for SCT templates 1-9 in FIG. 2B. In other examples, the amino acid substitution includes a lysine at an amino acid position corresponding to amino acid 227 of SEQ ID NO: 3 (e.g., D227K), an alanine at an amino acid position corresponding to amino acid 228 of SEQ ID NO: 3 (e.g., T228A), a valine at an amino acid position corresponding to amino acid 245 of SEQ ID NO: 3 (e.g., A245V), or any combination of two or more thereof. In some embodiments, the HLA α chain includes one or more of H74L, Y84C, Y84A, A139C, D227K, T228A, and A254V, with the amino acid positions corresponding to those of SEQ ID NO: 3.

In some examples, the peptide is an antigen peptide or a placeholder peptide. In some examples, the antigen peptide is selected from a tumor-associated peptide, a neoantigen peptide, an autoimmune peptide (e.g., a “self” peptide), a fungal peptide, a bacterial peptide, and a viral peptide. Exemplary peptides are discussed in Section II.

In some embodiments, soluble human-glycosylated MHC Class I SCT proteins are assembled as a stable multimer. In particular examples, the soluble human-glycosylated MHC Class I SCT proteins are assembled as stable tetramers. In some embodiments, assembly of stable multimers (such as tetramers) is carried out using biotinylated SCTs.

In one example, biotinylated SCT monomers are tetramerized with fluorophore-labeled streptavidin (such as streptavidin-phycoerythrin). In other examples, biotinylated SCT monomers are tetramerized using a custom streptavidin-DNA conjugate that allows for subsequent binding to complementary ssDNA-biotin molecules, for example affixed to streptavidin-coated beads. In a further example, SCT monomers are conjugated onto 10×-compatible DNA barcoded dextramers. These dextramers may also be labeled with fluorophores and therefore may be used after SCT conjugation in the same manner for flow cytometry as SCT-tetramers described above.

Also provided are libraries of the soluble human-glycosylated MHC Class I SCT proteins, as monomers or stable multimers (such as tetramers). In some embodiments, the library includes 2 or more, such as 2-2500 (for example, 2-25, 5-50, 10-100, 20-200, 75-150, 200-400, 300-500, 400-600, 500-750, 600-800, 700-1000, 1000-1500, 1250-1750, 1500-2000, or 2000-2500) soluble human-glycosylated MHC Class I SCT proteins. In some examples, the library of soluble human-glycosylated MHC Class I SCT proteins includes a plurality of SCTs that differ in the HLA heavy chain, the peptide, or both.

In additional embodiments, the stable multimers are attached to a solid support, such as a polymer, a flat surface, a bead, or a nanoparticle scaffold. In one non-limiting example, the solid support is a magnetic bead (such as Dynabeads). In some examples, a library including a plurality of solid supports (such as beads or nanoparticles) is provided, each including a different SCT multimer that is attached or linked to the support. In some embodiments, biotinylated SCT monomers or tetramers are incorporated onto a scaffold containing streptavidin, such as a streptavidin-coated bead or nanoparticle or a streptavidin-coated surface (such as a multi-well plate).

### IV. Methods of Use

Also disclosed herein are methods of using the disclosed MHC Class I SCTs. The methods include identifying an antigen-specific CD8⁺ T cell. In some embodiments, the methods further include identifying the T cell receptor (TCR) of the antigen-specific T cell, and in some examples, producing a population of T cells that express the identified TCR. In further embodiments, the population of T cells may be administered to a subject in need thereof.

In some embodiments, the methods include screening a population of T cells (e.g., contacting a population of T cells) with one or more stable multimers of a soluble human glycosylated MHC Class I SCT protein disclosed herein. In some examples, the population of T cells is contacted with a library of stable multimers, for example including a plurality of different SCT multimers, wherein each of the SCT multimers includes a different peptide sequence (such as a plurality of different peptide antigens and/or a plurality of HLA α chains). This allows detection of one or more T cells in the population that are reactive to a particular peptide, which are referred to in some examples as “antigen-specific T cells.” In some examples, the T cells screened with the SCTs are produced from peripheral blood mononuclear cells (PBMC) stimulated with the peptides included in the plurality of the SCTs.

The reactive T cells in the population can be sorted and captured, for example using flow cytometry. In some examples, the reactive T cells are expanded in vitro using cell culture methods known to one of skill in the art. In some embodiments, the T cells are analyzed to identify the TCR expressed in the reactive cells. In one example, the TCR is sequenced, for example, using next generation sequencing methods (for example, bulk sequencing or 10× single-cell sequencing).

The identified TCR is cloned into an expression vector, and a population of T cells is transformed with the expression vector encoding the TCR, to produce a population of T cells (e.g., CD8 T cells) expressing the TCR. Methods of transforming T cells to express a heterologous protein (such as the identified TCR) are known to one of ordinary skill in the art. This population of transformed T cells may be administered to a subject in need thereof. Methods of adoptive cell transfer are known to one of ordinary skill in the art. In some examples, the T cells expressing the TCR are reactive to a tumor-associated antigen or a neoantigen, and are administered to a subject with cancer. In other examples, the T cells expressing the TCR are reactive to a viral or bacterial antigen and are administered to a subject infected with the virus or bacteria.

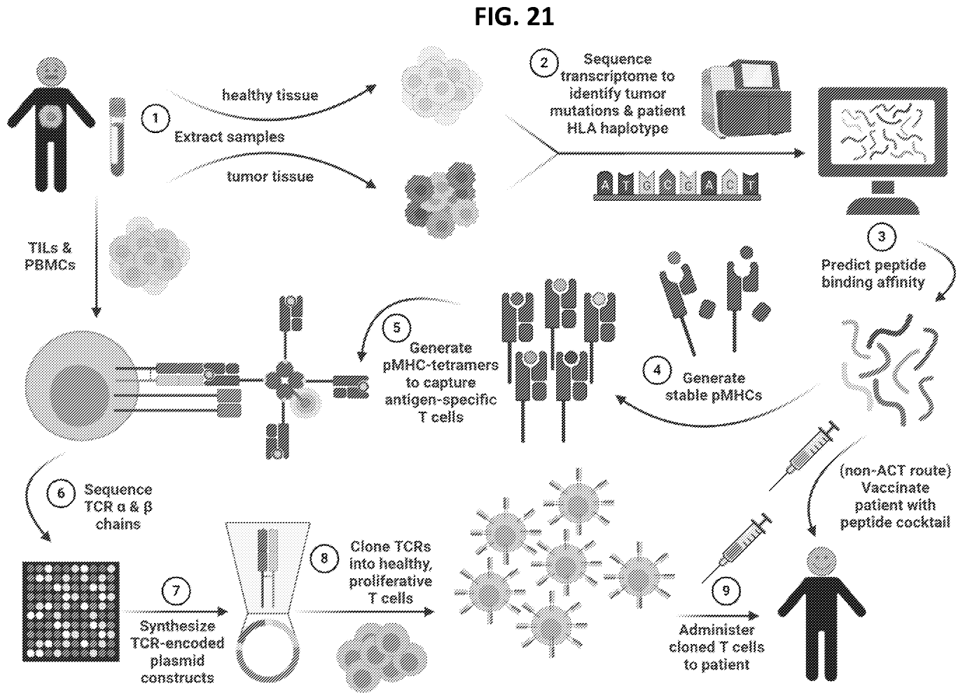

In some examples, the peptides used to generate the SCTs and screen the population of T cells are from a subject, such as a subject with cancer. In some examples, the population of T cells expressing the identified TCR are also from the subject (for example, are autologous T cells). A specific embodiment of the methods is illustrated in FIG. 21 and described in Example 8. However, one of ordinary skill in the art will recognize that modifications to these methods are possible.

### EXAMPLES

The following examples are provided to illustrate certain particular features and/or embodiments. These examples should not be construed to limit the disclosure to the particular features or embodiments described.

### Example 1

### Materials and Methods

SCT Template Production: Class I SCT-encoded plasmids were constructed using a combination of Gibson assembly and restriction enzyme digest methods for insertion into pcDNA3.1 Zeo(+) plasmid (Thermo Fisher Scientific) (FIG. 1A). Briefly, the SCT inserts were designed to be modular to allow for any choice of L1 to be paired with any choice of HLA allele. Because β2m has no allelic variation in the human species, the SCT was split into two Gibson assembly fragments within this region to allow for decoupling of L1 from HLA. Fragments were purchased from Twist Bioscience, PCR-amplified with KOD HotStart Hi-Fi polymerase (MilliporeSigma), and joined together by Gibson assembly using NEBuilder HiFi DNA Assembly Master Mix (New England Biolabs). The PCR-amplified Gibson product's flanking regions were digested by EcoRI and XhoI (New England Biolabs) to be ligated into the MCS region of pcDNA3.1 at the same enzyme recognition sites (FIG. 1B). Codon optimization was applied to the designed fragments under three considerations: 1) selection of only highly prevalent codons in the human species, 2) avoidance of continuous gene segments (24+ bp) where GC content is above 60% (to avoid error rates during synthesis), and 3) avoidance of key recognition cut sites within the fragments, which must only exist at the flanks of the Gibson product for insertion into pcDNA vector. This strategy was initially used successfully across three HLA alleles (A*01:01, A*02:01, A*03:01). Subsequently, the design of the second fragment (encoding HLA allele) was automated with a Python script, encompassing all aforementioned design criteria and accounting for all alleles from Class I HLA-A, B, C loci. The protein sequences of each HLA allele were obtained from an FTP server hosted by The Immuno Polymorphism Database (ftp.ebi.ac.uk/pub/databases/ipd/imgt/hla/fasta/). To date, all existing Class I HLA sequences from the IMGT database have been converted in this manner into ready-to-order DNA sequences. From these sequences, at least 40 unique plasmid templates have been constructed, encompassing 24 HLA-A, HLA-B, and HLA-C alleles.

SCT Peptide Library Production: A PCR-facilitated approach was implemented to enable high-throughput substitution of peptides into SCT-encoded plasmids. Extension PCR methods was chosen among other potential approaches after consideration of cost, ease-of-use, and flexibility for various L1 choices coupled next to the plasmid (FIG. 1C). Briefly, for any given peptide substitution, a peptide-encoded reverse primer (binding to the signal sequence upstream of peptide region) and a forward primer (binding to L1 downstream of peptide region) is required. The peptide-encoded primer varies for any given peptide, while the forward primer remains fixed across all peptide elements (unless one chooses to use a different L1/HLA template plasmid). In this manner, an SCT plasmid library, encompassing n peptides and m templates, requires the purchase of n+m total primers. Extension PCR was conducted with KOD Hot Start polymerase (MilliporeSigma). The product was phosphorylated and ligated with a mixture of T4 Polynucleotide Kinase and T4 DNA Ligase, and then template DNA was digested with DpnI (New England Biolabs). The peptide-substituted plasmids were then transformed into One Shot TOP10 Chemically Competent E. coli (Thermo Fisher Scientific). Plasmids were verified by Sanger sequencing using a Python script prior to use in transfection.

SCT Expression: Purified SCT plasmids were transfected into Expi293 cells (Thermo Fisher Scientific) within 24-well (2.5 ml capacity) plates. Briefly, 1.25 μg of plasmid was mixed with 75 μl Opti-MEM reduced serum media. 7.5 μl of ExpiFectamine Reagent was mixed with 70 μl Opti-MEM reduced serum media, incubated at room temperature for 5 minutes, and combined with the plasmid mixture. After a 15-minute room temperature incubation, the solution was added to 1.25 ml of Expi293 cells at 3 million cells/ml into a 24-well plate, which was then shaken at 225 RPM at 37° C. in 8% CO₂ overnight. Twenty hours later, a solution containing 7.5 μl of ExpiFectamine Transfection Enhancer 1 and 75 μl of ExpiFectamine Transfection Enhancer 2 was added to each well. The plate was kept on the shaker using aforementioned settings for a total of 4 days from start of transfection. The supernatant of the transfection solution was collected and filtered through 0.22 μm PVFD membrane syringe filters (MilliporeSigma) prior to yield analysis via SDS-PAGE. The supernatant solutions of SCTs which expressed at high yield were concentrated down to 200 μl PBS using 30 kDa centrifugal filter units (Amicon) and subsequently biotinylated with BirA enzyme kit (Avidity) overnight. The biotinylated SCTs were then purified with HisTag resin tips (Phynexus) and desalted back into PBS buffer with Zeba 7KMWCO spin desalting columns (Thermo Fisher Scientific). For long-term storage, the SCTs were re-suspended into 20% glycerol w/v prior to storage at −20° ° C.

SCT Yield Characterization: After 4 days of transfection, a 15 μl solution containing 3:1 mix of transfection supernatant and Laemmli buffer with 10% β-mercaptoethanol was denatured at 100° ° C. for 10 minutes, and subsequently loaded into Bio-Rad Stain-Free gels for SDSPAGE (200V, 30 minutes). A reduced, purified WT1 (RMFPNAPYL; SEQ ID NO: 1) A*02:01 SCT sample in 20% glycerol PBS solution (containing approximately 2 μg) was run in each gel to serve as a positive control and intensity reference for relative protein yield calculation. Images were obtained using a Bio-Rad ChemiDoc MPgel imaging system (manual settings: 45 seconds UV activation, 0.5 second exposure). To identify a consistent approach for analyzing SCT expression, a custom Python script was developed specifically for the analysis of SCT proteins run on Stain-Free gels (Bio-Rad). The script allows for user-defined selection of protein bands of interest, and provides background reduction and uniform normalization of SCT yield across all gels given the consistent use of a control protein lane. The accuracy of this approach was measured by SDS-PAGE of titrated, pre-quantified samples of purified SCTs to demonstrate a 99% correlation between true protein A280 concentration (as measured by NanoDrop 8000 Spectrophotometer) and quantified relative band intensity. SCTs which expressed above an established cutoff for yield were selected for subsequent biotinylation and purification steps.

Thermal Stability Characterization: SYPRO™ Orange Protein Gel Stain was purchased from ThermoFisher Scientific and diluted with water to give a 100× working solution. To each 19 μl aliquot of Class I SCT protein solution (diluted to 10 μM, if possible), 1 μl of the 100× dye solution was added. A Bio-Rad thermal cycler equipped with a CFX96 real-time PCR detection system was used in combination with Precision Melt Analysis software to obtain melting curves of each SCT sample. Thermal ramp settings were 25° C. to 95° C., 0.2° C. per 30 seconds.

Peptide Stimulation: The thawed PBMCs were incubated in complete R10 media (500 ml of RPMI 1640; 50 mL Heat-inactivated FBS; 5 ml of Pen/strep (100 U/mL penicillin and 100 μg/mL streptomycin); 1× GlutaMAX) by adding 1 μM of peptide and anti-CD40 antibody (1 μg/mL) for 16 hrs. On the next day, the PBMCs were washed and stained with Annexin V-BV421 (1 μg/mL), CD8-FITC antibody (1 μg/mL) and CD137-PE antibody (1 μg/mL) for 10 mins at 4° C. Activation-induced expression of CD137 by peptide stimulation permits the sorting of antigen specific T-cells into tubes using FACS sorter equipment.

SCT Multimer Formation: Biotinylated SCT monomers have been successfully used in at least three different formats. First, they have been tetramerized with Streptavidin-Phycoerythin (PE) (BioLegend) for use as conventional flow cytometry staining reagents. Second, they have been tetramerized with a custom-made streptavidin-DNA conjugate to allow for subsequent binding onto complementary ssDNA-biotin molecules affixed on streptavidin-coated magnetic Dynabeads (Thermo Fisher Scientific). These reagents can be utilized in a nanoparticle-nucleic acid cell sorting platform (NP-NACS) (Peng et al., Cell Reports 28:2728-2738, 2019), which allows for enhanced pMHC-TCR avidity and microfluidic-guided extraction and analysis of antigen-specific T cells. The SCT monomers have been conjugated onto 10×-compatible DNA barcoded dextramers (Immudex). These reagents enable coupling of the antigen-specific identity (DNA barcoded onto dextramers) of a captured CD8 T cell and its corresponding TCR α and β chain sequences (single-cell mRNA sequencing).

### Example 2

### Expression of SCT Library

The initial SCT library consisted of 18 HLA-A*02:01 antigens derived from various sources (Table 1). To identify candidate L1/HLA mutations to introduce into the SCT, a literature survey was carried out for engineered improvements made to SCT design. Three generations of L1-HLA combinations (closed groove (wild-type HLA Y84), open groove (HLA Y84A), and thiol linker (HLA Y84C)) have been previously explored and shown to demonstrate gradual improvements in pMHC stability. These three generations were implemented into five unique designs, abbreviated D1 (L1=(GGGGS)₃ (SEQ ID NO: 137); closed groove), D2 (L1=(GGGGS)₃ (SEQ ID NO: 137); open groove), D3 (L1=GCGGS(GGGGS)₂ (SEQ ID NO: 138) thiol linker), D4 (L1=GGCGS(GGGGS)₂ (SEQ ID NO: 138); thiol linker), and D5 (L1=GCGAS(GGGGS)₂ (SEQ ID NO: 139); thiol linker) (FIGS. 2A and 2B). Designs which contained a cysteine in the linker (D3-D5) also incorporated the Y84C mutation in the HLA subunit to enable dithiol linkage. Next, an orthogonal HLA mutation, H74L, was implemented into three of the templates (D6-D8). The H74L mutation forms a portion of the C pocket in the peptide binding groove of the HLA subunit and has been reported to facilitate peptide loading and pMHC immunogenicity, so its inclusion may improve overall pMHC stability and function. The final design (D9, termed DS-SCT) includes a paired Y84C-A139C mutation to the HLA binding pocket that could introduce further stabilization to refolded pMHC construct.

TABLE 1
Peptides for SCT library template optimization
studies

| No. | Peptide | Protein | SEQ ID NO: |
|---|---|---|---|
| 1 | YMLDLQPET | E7 (PPV-9) | 6 |
| 2 | YMLDLQPETTDL | E7 (PPV-9) | 7 |
| 3 | LLMGTLGIV | E7 (PPV-9) | 8 |
| 4 | TLGIVCPI | E7 (PPV-9) | 9 |
| 5 | SLLQHLIGL | MART | 10 |
| 6 | VLQELNVTV | Myeloblastin | 11 |
| 7 | SVAPALALFPA | LB-ADIR-1F | 12 |
| 8 | FLKANLPLL | MTG8b | 13 |
| 9 | KLSAMQAHL | Foxp3 | 14 |
| 10 | LQLPTLPLV | Foxp3 | 15 |
| 11 | VLHDDLLEA | HA-1/A2 | 16 |
| 12 | VFEEPEDFL | Foxp3 | 17 |
| 13 | AIQDLCLAV | nucleophosmin | 18 |
| 14 | AIQDLCVAV | nucleophosmin | 19 |
| 15 | ALYVDSLFFL | PRAME | 20 |
| 16 | RMFPNAPYL | WT1 | 1 |
| 17 | SLLMWITQV | NY-ESO-1 | 21 |
| 18 | ELAGIGILTV | MART-1 | 2 |

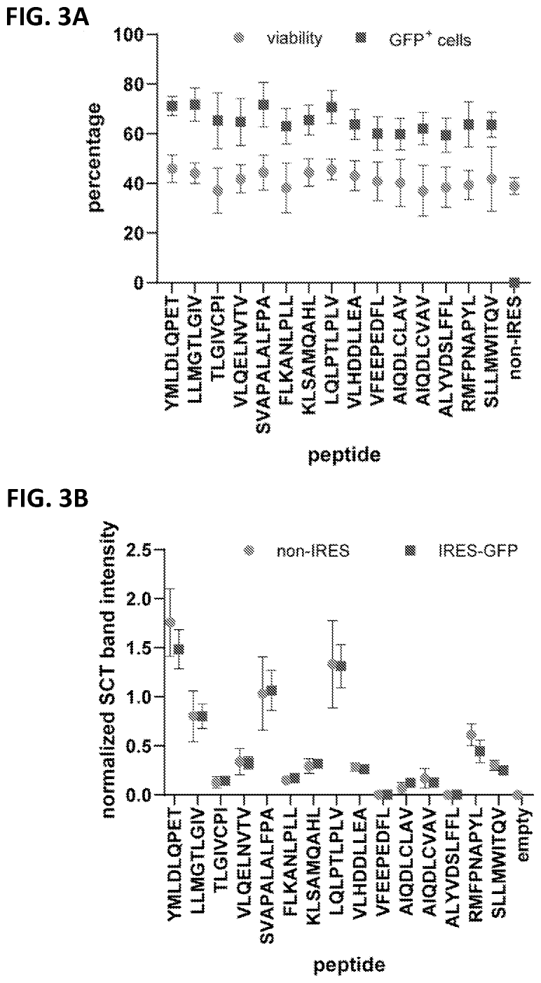

This 162-element plasmid library, encompassing nine HLA templates and 18 peptides, was transfected into Expi293 cells (FIG. 2B). Reduced SDS-PAGE analysis of the SCT protein bands revealed significant variations in protein yield that was dependent on peptide and template (FIG. 2B). To decouple the effect of transfection efficiency on SCT yield, a subset of the library under design D3 was further modified to incorporate an IRES-GFP sequence, such that regardless of peptide identity or degree of SCT expression, transfected cells would be induced to express intracellular GFP. Flow cytometry-based detection of GFP-positive cells indicated that the degree of transfection efficiency was approximately uniform (70%) across all tested SCT constructs (FIG. 3A). A biological triplicate of this subset, with and without the IRES-GFP insert, was conducted to demonstrate that the peptide-dependent SCT yield variations are consistent (FIG. 3B). The three H74L mutation templates among the library generally demonstrated improved protein expression relative to their wild-type counterparts, and the templates making use of thiol linkers produced the highest overall yields of SCTs (FIG. 2B). In some cases, such as the peptide AIQDLCLAV (SEQ ID NO: 18), SCT expression could only be obtained with the D8 template, which incorporates both H74L and thiol linker features, or with the D9 template, possibly due to stability at the F pocket conferred by the dithiol mutation. There was a slight upward shift of the SCT band for VLQELNVTV (SEQ ID NO: 11), indicating increased mass due to the NXT glycosylation consensus sequence in the peptide region (FIG. 2B). This phenomenon is absent in assembly methods which require exogenous introduction of peptide and shows that SCTs undergo biological protein processing pathways prior to secretion. Thus, SDS-PAGE analysis of this library revealed that SCT expression is dependent on the choice of peptide and backbone template, and produces protein containing post-translational modifications.

SCTs which expressed above a yield threshold were subsequently HisTag-purified into PBS buffer at pH 7.4 for thermal shift assays. The measured Tₘ values were within expected values of reported SCTs compared to native pMHC counterparts, providing a trend of increased stability for the same peptide from wild-type groove (D1 & D6) to open groove (D2 & D7) to thiolated linker/groove (D3, D4, D5, D8, D9) (FIG. 2C). SCT thermal stability for each peptide was also higher for H74L variants than wild-type counterparts. For some peptides (such as AIQDLCLAV (SEQ ID NO: 18) or FLKANLPLL (SEQ ID NO: 11)) in which SCTs expressed only for some templates, two distinct Tₘ values were detected, the lower of which may indicate an improperly folded SCT species.

### Example 3

### SCT Functional Assay Against Tumor-Associated Antigen

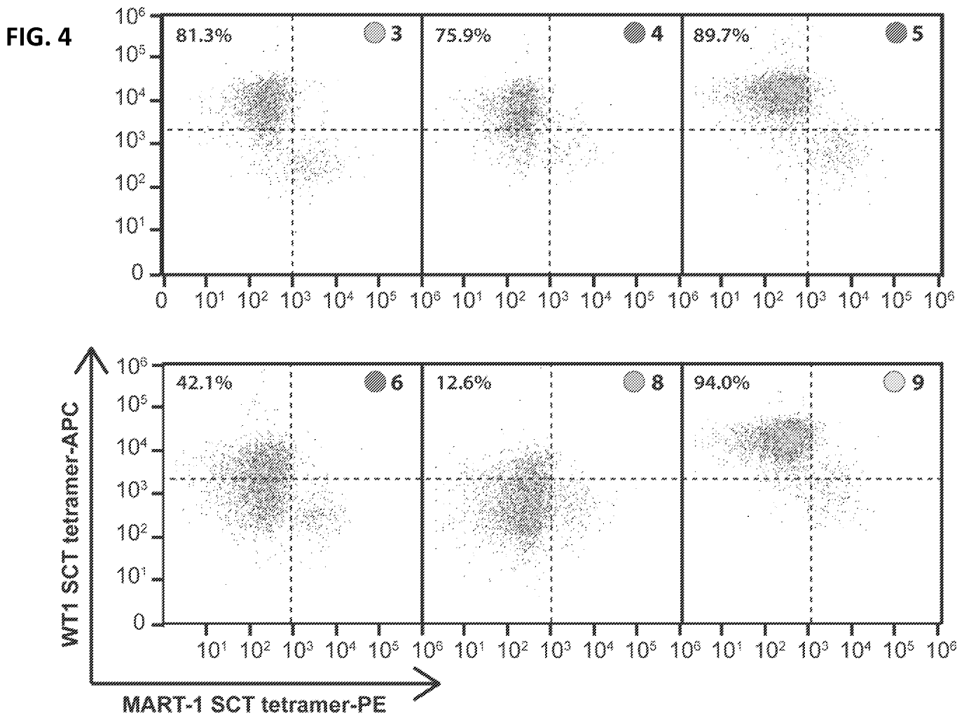

To validate the functionality of the SCT constructs, SCT binding efficiencies were assessed across various designs against known TCRs. For the Wilms Tumor 1 (WT1) peptide (RMFNAPYL; SEQ ID NO: 22), the binding of this series of six SCTs (D1, D2, and D7 yields were too low for use) were assessed against the WT1-specific C4 TCR, which has been characterized by others for reactivity to the peptide in vivo (FIG. 4). Expressed WT1 SCTs were purified and used in binding assays against a 95/5 mixed population of C4 TCR-transduced and MART-1-specific F5 TCR-transduced Jurkat cells. Significant differences in the degree of binding by WT1 SCTs to WT1-specific Jurkat cells was observed. The H74L SCT variants (D6 and D8) displayed the poorest performance, capturing approximately two-fold fewer cells within the gates compared to the wild-type H74 counterparts. The DS-SCT variant for WT1 demonstrated the best binding efficiency in the same assay against C4 TCR-transduced Jurkat cells, capturing 97.3% of the WT1-specific cell population. A similar assay was performed for the MART-1 epitope against a pure population of F5 TCR-transduced TCR Jurkat cells to produce similar results. Consequently, the DS-SCT template was used for peptide libraries in future experiments.

### Example 4

### SCT Functional Assay Against Viral Antigens

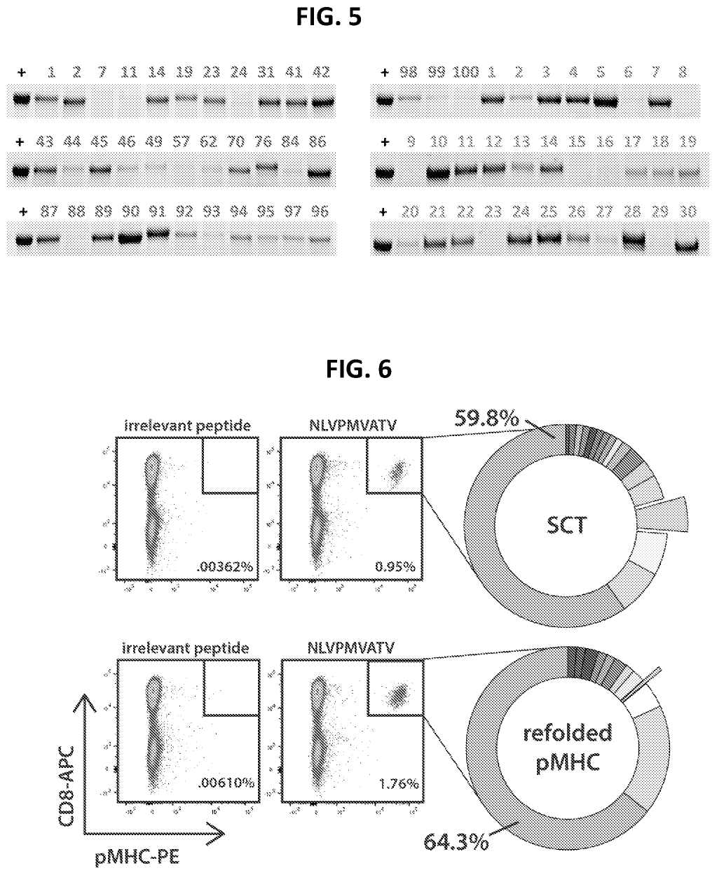

To extend the platform toward use cases in infectious disease, a small SCT library targeting common viral epitopes was expressed. Plasmid templates against 66 total A*02:01 or A*24:02 viral epitopes commonly reported in the literature were constructed (Tables 2 and 3). Similar to the previous library, all plasmids displayed peptide-dependent SCT expression (FIG. 5). The SCTs were ranked by protein expression, and ten epitopes derived from common viral strains (CMV, EBV, influenza, and rotavirus) from each of two HLA types and resulting in the highest SCT expression were selected for further use in identification of antigen-specific specific T cells. PBMCs obtained from HLA-matched healthy donors were stimulated with corresponding peptide pools containing these epitopes over approximately one month with weekly re-stimulation to induce expansion of peptide-specific clonotypes. For each donor, ten lines of cells from the same PBMCs were stimulated under these conditions. Peptide-stimulated and expanded T cell lines were sorted with SCT tetramers and displayed significantly higher quantities of tetramer-bound populations compared to their unstimulated counterparts for most peptides. This demonstrates that SCTs can capture cognate TCRs which recognize the same epitope bound onto native, surface-bound MHC complexes.

TABLE 2
A*02:01 viral antigens

| | | | SEQ |
|---|---|---|---|
| ID | Peptide | Antigen source | ID NO: |
| 1 | LLFGYPVYV | HTLV-1 Tax | 23 |
| 2 | KLVALGINAV | HCV | 24 |
| 7 | GLCTLVAML | EBV-BLMF1 | 25 |
| 11 | WLSLLVPFV | HBV-SAg | 26 |
| 14 | YVLDHLIVV | EBV-BRLF1 | 27 |
| 19 | SITEVECFL | Human polyomavirus 2 | 28 |
| 23 | FLLSLGIHL | HBV | 29 |
| 24 | GILGFVFTL | Flu-M1 | 30 |
| 31 | SLFNTVATL | HIV gag | 31 |
| 41 | YLLFEVFDV | AdV11 Hexon | 32 |
| 42 | LLFEVFDVV | AdV11 Hexon | 33 |
| 43 | YVLFEVFDV | AdV11 Hexon | 34 |
| 44 | FLDKGTYTL | EBV BALF4 | 35 |
| 45 | YLQQNWWTL | EBV-LMP1-2 | 36 |
| 46 | YLLEMLWRL | EBV-LMP1-1 | 37 |
| 49 | FLYALALLL | EBV-LMP1-2 | 38 |
| 57 | VLEETSVML | CMV-IE1 | 39 |
| 62 | TLNAWVKVV | HIV gag | 40 |
| 70 | AIMDKNIIL | Influenza NS1 | 41 |
| 76 | KLIANNTRV | M. tuberculosis Ag85A | 42 |
| 84 | ALWALPHAA | Varicella-zoster | 43 |
| | | IE62 593-601 | |
| 86 | NLVPMVATV | CMV-pp65 | 44 |
| 87 | FMYSDFHFI | Influenza A | 45 |
| 88 | YLLPGWKL | Rota-VP3 | 46 |
| 89 | NMLSTVLGV | Flu-PB1 | 47 |
| 90 | SLMDPAILTSL | Rota-VP1 | 48 |
| 91 | TLLANVTAV | Rota-VP6 | 49 |
| 92 | FMDILTTCVET | CMV-IE1-2 | 50 |
| 93 | QMWQARLTV | CMV-pp65-2 | 51 |
| 94 | SLISGMWLL | Rota-VP2-1 | 52 |
| 95 | LLNYILKSV | Rota-VP7-1 | 53 |
| 96 | LMNGQQIFL | CMV-pp65-3 | 54 |
| 97 | FLDSEPHLL | Rota-NSP1 | 55 |
| 98 | ALWGPDPAAA | Proinsulin precursor | 56 |
| | | 15-24 | |
| 99 | TLDYKPLSV | EBV BMRF1 | 57 |
| 100 | CLGGLLTMV | EBV-LMP2A | 58 |

TABLE 3
A*24:02 viral antigens

| | | | SEQ |
|---|---|---|---|
| ID | Peptide | Antigen source | ID NO: |
| 1 | TYFNLGNKF | AdV 11 Hexon (37-45) | 59 |
| 2 | VYSGSIPYL | AdV 11 Hexon (696-704) | 60 |
| 3 | TYFSLNNKF | AdV 5 Hexon (37-45) | 61 |
| 4 | DYNFVKQLF | EBV BMLF1 (320-328) | 62 |
| 5 | TYPVLEEMF | EBV BRLF1 (198-206) | 63 |
| 6 | RYSIFFDYM | EBV EBNA3A (246-254) | 64 |
| 7 | TYSAGIVQI | EBV EBMA3B (217-225) | 65 |
| 8 | IYVLVMLVL | EBV LMP2 (222-230) | 66 |
| 9 | PYLFWLAAI | EBV LMP2 (131-139) | 67 |
| 10 | TYGPVFMSL | EBV LMP2 (419-427) | 68 |
| 11 | TYGPVFMCL | EBV LMP2 (419-427) | 69 |
| 12 | EYLVSFGVW | HBV core (117-125) | 70 |
| 13 | KYTSFPWLL | HBV pol (756-764) | 71 |
| 14 | QYDPVAALF | HCMV pp65 (341-349) | 72 |
| 15 | EYVLLLFLL | HCV E2 (717-725) | 73 |
| 16 | PFHCSFHTI | HHV-6B U54 (267-275) | 74 |
| 17 | RYLRDQQLL | HIV env gp160 (584-592) | 75 |
| 18 | RYLKDQQLL | HIV env (67-75) | 76 |
| 19 | RYPLTFGW | HIV nef (134-141) | 77 |
| 20 | VYDFAFRDL | HPV16 E6 (49-57) | 78 |
| 21 | FFQFCPLIF | HTLV-1 Env (43788) | 79 |
| 22 | LFGYPVYVF | HTLV-1 Tax (43819) | 80 |
| 23 | PYKRIEELL | HTLV-1 Tax (187-195) | 81 |
| 24 | SFHSLHLLF | HTLV-1 Tax (301-309) | 82 |
| 25 | YYLEKANKI | Influenza PA (130-138) | 83 |
| 26 | SYLIRALTL | Influenza PB1 (216-224) | 84 |
| 27 | RYTKTTYWW | Influenza PB1 (430-438) | 85 |
| 28 | SYINRTGTF | Influenza PB1 (482-490) | 86 |
| 29 | RYGFVANF | Influenza PB1 (498-505) | 87 |
| 30 | TYQWIIRNW | Influenza PB2 (549-557) | 88 |

To further assess functional capacity of the SCTs, the sequences of the CDR3 regions from TCR α and β chains captured by SCT dextramers were queried. A healthy A*02:01 donor was identified to have positive reactivity against the peptide NLVPMVATV (SEQ ID NO: 44), which is derived from human cytomegalovirus (CMV) pp65 protein. This SCT element and its folded pMHC counterpart were used to sort for CMV-specific T cells from the donor PBMCs (FIG. 6). 10× single-cell sequencing of the sorted population revealed a similar distribution of antigen-specific clones captured by the two reagents. As seen in Table 4, Levenshtein distances (LD) of the CDR3α and CDR3β chains against a public database (VDJdb) were low, indicating high similarity between the detected CMV-specific TCR chains and those previously reported. Two paired clones (red and light orange wedges in FIG. 6) contained CDR3α chains exactly matching literature results (LD=0). An additional clone (light green wedge in FIG. 6) contained an α/β pair for which both chains have been reported as CMV-specific, and was captured by the SCT at a ten-fold higher frequency. These results indicate that SCT tetramers have at least similar flow cytometry performance to the gold standard of folded pMHCs.

TABLE 4
TCR CDR3α and CDR3ß sequences of the twelve most frequently
captured clonotypes from SCT tetramer

| CDR3α | LD | CDR3β | LD |
|---|---|---|---|
| CATVGTASKLTF (SEQ ID | 5 | CASSLWLNEQFF (SEQ ID | 2 |
| NO: 89) | | NO: 101) | |
| CARNTGNQFYF (SEQ ID | 0 | CASSPKTGASYGYTF (SEQ | 2 |
| NO: 90) | | ID NO: 102) | |
| CVVGYGQFYF (SEQ ID NO: | 4 | CASSFVSFDEQFF (SEQ ID | 4 |
| 91) | | NO: 103) | |
| CAGPMKTSYDKVIF (SEQ | 0 | CASSSAYYGYTF (SEQ ID | 0 |
| ID NO: 92) | | NO: 104) | |
| CAASRKGSNYKLTF (SEQ | 5 | CASSADSYGANVLTF (SEQ | 4 |
| ID NO: 93) | | ID NO: 105) | |
| CAVRWGGKLSF (SEQ ID | 5 | CSVDPGHTGEKLFF (SEQ ID | 6 |
| NO: 94) | | NO: 106) | |
| CAEIPNYGGSQGNLIF (SEQ | 0 | CASSLVGGRHGYTF (SEQ | 2 |
| ID NO: 95) | | ID NO: 107) | |
| CAESSASKIIF (SEQ ID NO: | 5 | CASSHDPTWGPGNTIYF | 6 |
| 96) | | (SEQ ID NO: 108) | |
| CAVRDRWSGGYQKVTF | 8 | CASSFGQGSSPLHF (SEQ ID | 4 |
| (SEQ ID NO: 97) | | NO: 109) | |
| CAVRVSGGYNKLIF (SEQ | 5 | CASSLETVNTEAFF (SEQ ID | 3 |
| ID NO: 98) | | NO: 110) | |
| CAVTLNNNAGNMLTF | 6 | CASSSFYDSNEKLFF (SEQ | 4 |
| (SEQ ID NO: 99) | | ID NO: 111) | |
| CALSPRTQGGSEKLVF | 4 | CASSLASPGHFTGELFF | 4 |
| (SEQ ID NO: 100) | | (SEQ ID NO: 112) | |

LD = Levenshtein distance to publicly reported CMV-specific clonotypes from VDJdb

### Example 5

### Enumeration of Antigen-Specific T Cells Against SARS-COV-2

This example was previously published (in a modified format) as Chour et al., medRxiv, doi.org/10.1101/2020.05.04.20085779, on May 8, 2020, incorporated herein by reference in its entirety.

### Methods

pMHCs were designed in the form of a plasmid-encoded single-chain trimer comprising a candidate SARS-COV-2-derived spike protein or Nsp3 epitope, β-2 microglobulin subunit of the MHC, and the human leukocyte antigen (HLA) subunit of the MHC. The optimized platform was utilized to express approximately 118 viable SCT constructs against the spike protein, and 75 against Nsp3. 88 of the spike SCTs and 75 of the Nsp3 SCTs were incorporated as tetramers into a nanoparticle nucleic acid cell sorting (NP-NACS) system to generate high-avidity TCR capture agents. Last, NP-NACS was applied toward the identification and analysis of antigen-specific T cells derived from blood draws of eight COVID-19 participants covering three HLA alleles of interest, as well as from four HLA-matched healthy donor PBMC samples.

Sample collection: All human samples (blood) were obtained after institutional approval and participant-written informed consent, as part of the Swedish Institute's INCOV trial to study COVID-19 participants. Peripheral blood mononuclear cells (PBMCs) were isolated and cryopreserved. They were collected from participants at up to three timepoints: T1 (diagnosis), T2 (4-5 days after diagnosis), and T3 (convalescence). 186 unique participant samples were submitted for HLA haplotyping (Cisco Genetics). Among all samples, we identified A*02:01, A*24:02, and B*07:02 alleles as the most prevalent, and therefore filtered for participants with these alleles for further analysis using SCT constructs.

SCT plasmid construction & protein expression: In order to build SARS-COV-2 SCT libraries, identified peptides were encoded into primers for insertion into template SCT plasmids (as discussed in Example 1). The peptide-substituted SCT plasmid libraries were subsequently transfected into Expi293 cells for approximately four days. Secreted SCT proteins were collected from the supernatant, biotinylated, and purified by HisTag column.

SCT multimer assays: SCT monomer libraries can be biotinylated and incorporated into standard tetramer scaffolds for various downstream assays. The SCT tetramers can then be assembled onto the surface of magnetic nanoparticles to form pMHC-nanoparticle (pNP) libraries for hemocytometry fluorescence microscopy assays. Furthermore, these SCTs can be used with Immudex Klickmer reagents to form dextramers for use in 10× single-cell sequencing experiments. pNP libraries are advantageous in that all analysis is done in solution, thus avoiding risks from aerosolized COVID-19 patient biospecimens. Prior work using the NP-NACS system highlights the enhanced sensitivity of this platform, which allows for its use with non-expanded CD8+ T cells directly extracted from PBMCs. However, enumeration of TCR sequences from captured cells is difficult, and requires further microfluidic adaptations to enable single-cell sequencing. Compared to NP-NACS, flow cytometry assays making use of SCT tetramers are higher throughput and can be combined with bulk sequencing assays to identify antigen-specific TCR sequences, but the degree of specific binding by tetramers is more difficult to resolve as one cannot visualize tetramer staining at the microscopic level. Dextramer/10× assays are utilized in a similar manner to tetramers for flow cytometry and allow for antigen-pairing of TCR sequences, but compared to the other approaches is relatively more expensive and lower throughput, enabling analysis of only up to 10,000 cells per run. In order to maximize confidence that sequenced TCRs are derived from antigen-specific T cells, the latter two assays worked with CD8+ T cells which had been expanded after either SCT capture or peptide stimulation.

Production of cysteine-modified streptavidin-DNA (SAC-DNA) conjugates: The SAC-DNA conjugate was produced as follows. Briefly, SAC was first expressed from the pTSA-C plasmid containing the SAC gene (Addgene). Before conjugation to DNA, SAC (1 mg/ml) was buffer exchanged to PBS containing Tris(2-Carboxyethyl) phosphine hydrochloride (TCEP, 5 mM) using Zeba desalting columns (Pierce). Then 3-N-Maleimido-6-hydraziniumpyridine hydrochloride (MHPH, 100 mM, Solulink) in DMF was added to SAC at a molar excess of 300:1. In the meantime, succinimidyl 4-formylbenzoate (SFB, 100 mM, Solulink) in DMF was added to 5′-amine modified ssDNA (500 μM) in a 40:1 molar ratio. After reacting at room temperature (RT) for 4 hours, MHPH-labeled SAC and SFB-labeled DNA were buffer exchanged to citrate buffer (50 mM sodium citrate, 150 mM NaCl, pH 6.0), and then mixed at a 20:1 ratio of DNA to SAC to react at RT overnight. SAC-DNA conjugate was purified using the Superdex 200 gel filtration column (GE health) and concentrated with 10K MWCO ultra-centrifuge filters (Millipore).

COVID SCT pNP library construction: Streptavidin-coated NPs (500 nm radius, Invitrogen Dynabeads MyOne T1) were prepared according to the manufacturer's recommended protocol for biotinylated nucleic acid attachment. These NPs were mixed with barcoded biotin-ssDNA (100 μM) at 1:20 volume ratio to obtain NP-DNA. Excess DNA was removed by washing the NPs three times. In parallel, the SCT monomer library was added to SACDNA at a 4:1 ratio to form the SCT tetramer-DNA. To generate fluorescent pNPs, equimolar amounts (in terms of DNA ratio) of NP-DNA and pMHC tetramer-DNA were hybridized at 37° C. for 20 min, along with 0.25 μl of 100 μM ssDNA bound to AlexaFluor 750, AlexaFluor 488, or Cy5 (IDT-DNA), and washed once with buffer (0.1% BSA, 2 mM MgCl2 PBS). The use of three dyes allows for multiplexing of up to three unique antigen pNPs per analysis. Typically, each NP-barcoded NACS analysis of <100,000 cells uses 2.5 μL of stock NPs (28.2 million particles total) per library element.

Preparation and isolation of CD8⁺ T cells from PBMC suspensions: PBMCs were thawed and incubated in RPMI 1640 media supplemented with 10% FBS and IL2 (100 U/mL) for overnight recovery at 37° ° C., 5% CO₂. Recovered cell viability was measured at >95% for all samples. CD8+ T cell population was negatively selected using the CD8+ T Cell Isolation Kit (Miltenyi Biotec, Bergisch Gladbach, Germany). Briefly, recovered cells were incubated with a biotinylated antibody cocktail that captures CD8-cells in PBMCs followed by streptavidin-coated microbeads. The untouched CD8⁺ T cells were separated in a 15 mL Falcon tube using an LS column. The tube containing CD8⁺ T cells was then centrifuged at 500 g for 5 minutes and the pellet was re-suspended in PBS buffer. For the multiplex cell labeling, CD8⁺ T cells were individually stained with Calcein Blue, AM (Thermo Fisher Scientific) or CellTracker™ Orange CMRA Dye (Thermo Fisher Scientific) at the concentration of 4 μM and 400 nM, respectively. After incubation for 10 minutes at 37° C. under 5% CO₂, cells were washed twice with PBS and re-suspended in a cell suspension buffer (0.1% BSA, 2 mM MgCl₂ in PBS).

Identification of antigen-specific CD8+ T cells by NP-NACS: The pNP library was combined into groups of three pNPs, with each pNP element in the group stained with one of three barcode dyes. From each pNP group, 7.5 μl was incubated with each aliquot of stained CD8⁺ T cells at RT for 30 minutes. Antigen-specific cells were enriched by magnetic pulldown and re-suspended into 6 μl of 0.1% BSA 2 mM MgCl₂ PBS buffer. Captured cells were then loaded into a 4-chip disposable hemocytometer (Bulldog-Bio). The entire area in the hemocytometer chip was imaged to obtain the total pulldown cell number. Identification of antigen-specific T cells, including the detection and exclusion of non-specific binding events, was conducted with cellSens Olympus software and R programming language.

Tetramer binding flow assay: For use of SCTs in tetramer format for flow assays, see Example 1. Use of SCTs in dextramer format for 10× also followed similar protocols, where streptavidin was replaced with Immudex dextramer/Klickmer reagents, and downstream protocols for staining and washing were identical. For 10× single-cell sample submission, manufacturer's recommendations and protocols were utilized.

### Results

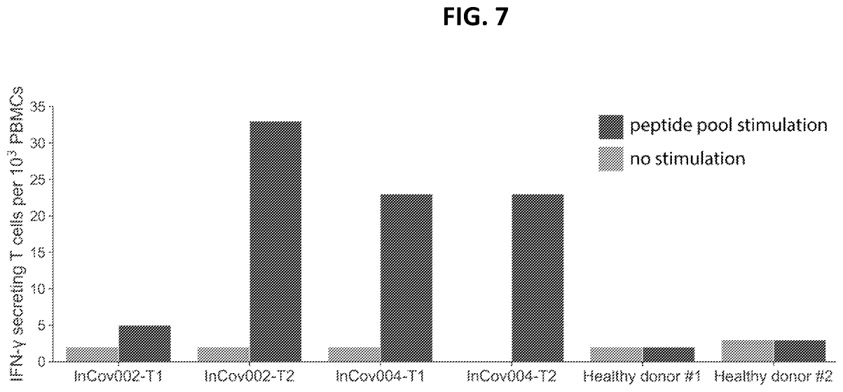

To broadly survey antigen-specific CD8+ T cell response against SARS-COV-2, PBMC samples of hospitalized COVID participants were collected from blood draws across three timepoints, starting from diagnosis (T1) to 4-5 days post-diagnosis (T2) to convalescence (T3). ELISpot assays based on stimulation with peptide pools of SARS-COV-2 structural proteins showed significantly increased IFN-γ production from two COVID participant PBMC samples versus health donor controls (FIG. 7). Among the INCOV participants, the increased IFN-signature primarily was detected at T2, indicating that an epitope-specific response against SARS-CoV-2 developed over time after infection.

Recent reports have indicated that the SARS-COV-2-specific T cell repertoire of hospitalized COVID patients consists of a large proportion of the exhausted phenotype and overall low CD8⁺ T cell counts correlated with disease severity. To enumerate the epitope landscape of SARS-COV-2-specific CD8⁺ T cells, including those which are potentially rare or exhausted, the PBMCs were probed directly instead of relying on a stimulation/expansion-based method. This approach prevents any potential bias against antigen-specific T cells with non-expandable phenotypes, which could skew the distribution of detected epitopes. To account for the absence of an expansion step, capture sensitivity was maximized using the NP-NACS platform, which affixes thousands of tetramers onto magnetic particles, enabling highly sensitive magnetic isolation and detection of clonal CD8⁺ T cells at frequencies as low as 0.001%. To capture as many antigen specificities as possible among unexpanded CD8⁺ T cells, capture breadth was broadened using the SCT platform to generate hundreds of pMHCs. 9- to 11-mer peptide sequences from a protein of interest were entered into the NetMHC4.0 binding prediction algorithm. For the spike protein, 96, 33, and 51 peptides were identified for HLA-A*02:01, B*07:02, and A*24:02 alleles, respectively, with 500 nM or stronger binding affinity (not shown).

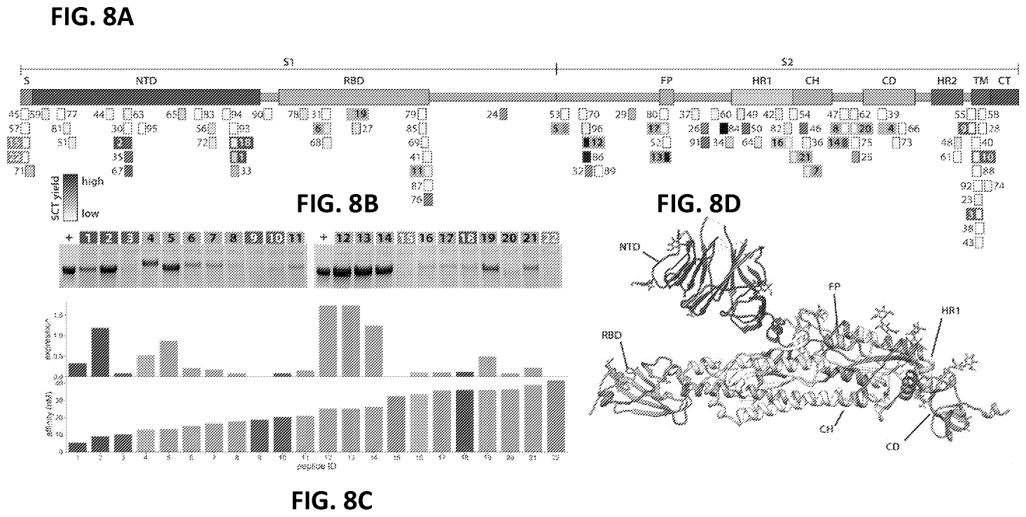

This filtered peptide list was used to develop pMHC-encoded plasmids using the SCT platform. The distribution of SCT protein expression for epitopes along the spike protein domain map were unique for each haplotype. A*02:01 SCTs showed relatively heterogeneous levels of expression for epitopes throughout all domains except TM (weak expression) (FIGS. 8A-8D). B*07:02 SCT expression showed preference for NTD, S1/S2 cleavage site, and parts of the S2 subunit, while highly expressed A*24:02 SCTs appeared to be concentrated around NTD, RBD, and TM regions. These distributions are partially skewed by artificial selection bias due to use of NetMHC4.0 as a filtering step prior to SCT production. Therefore, the expression of these SCTs to some degree are a reflection of the prediction strength of the algorithm. Additionally, the results may be seen as an interpretation of the biological differences that exist across the HLA alleles. Differences in hydrophilic/hydrophobic preference within each HLA's binding will bias the stability of each pMHC construct for certain peptide motifs found in the spike domains.

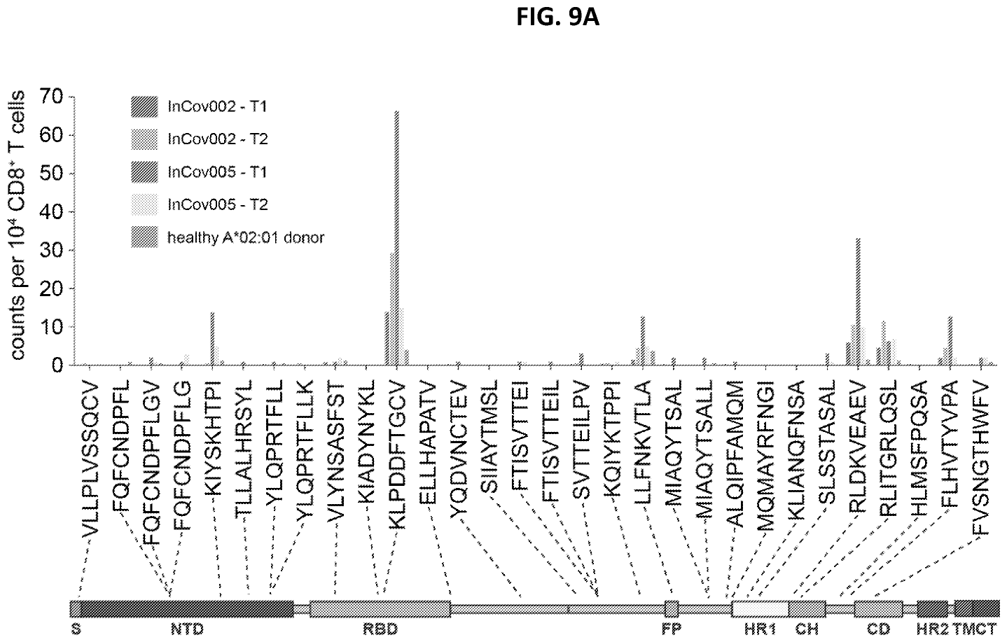

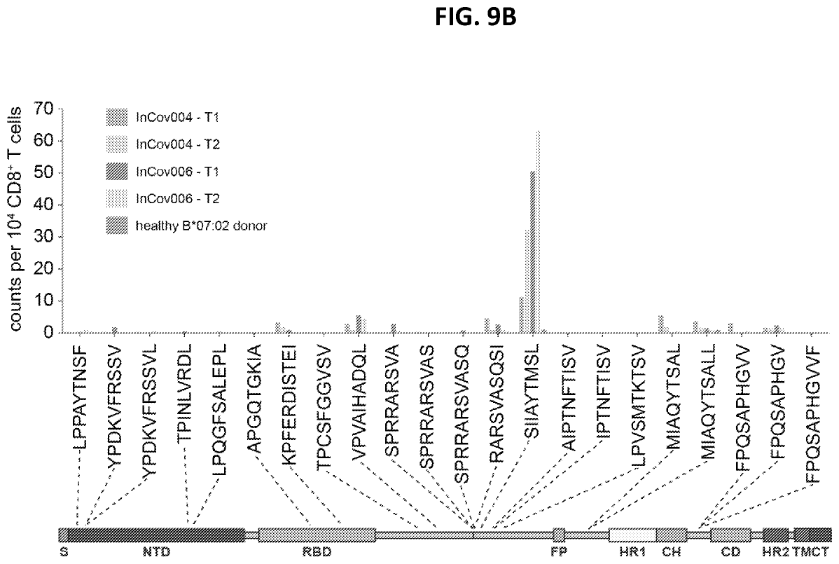

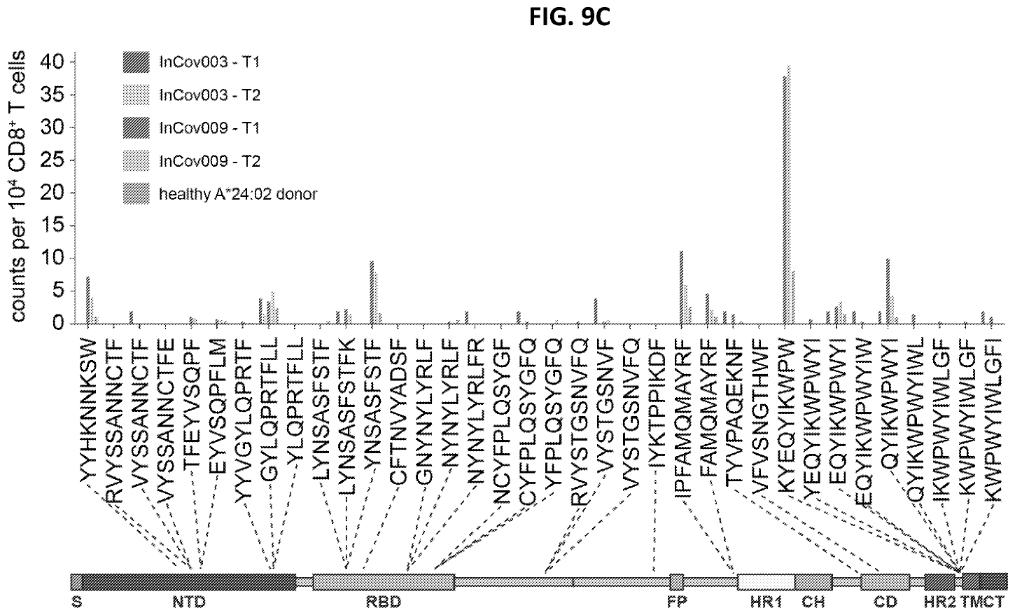

SCT multimers can identify antigen-specific T cells from healthy and COVID-19 donors: The highest expressing SCTs from each of the three libraries were utilized as NP-NACS reagents to identify antigen-specific T cells among COVID PBMCs from two participants and at least one healthy control per haplotype (FIG. 9). For each HLA haplotype, the NP NACS assay was able to identify antigen-specific T cells against a shared subset of epitopes per library, regardless of disease state of the samples. However, COVID participants contained significantly higher frequencies of antigen-specific T cells against each of the top epitopes relative to their healthy controls. These shared immunodominant epitopes were detected at both time points of sample collection for the COVID participants with variations in relative frequency for each, indicating perhaps fluctuations in clonotype expansion against each epitope throughout the immune response. These findings suggest that immunodominant epitopes are present among individuals of the same HLA haplotype, even among healthy controls, and that the degree of detection evolves throughout the course of disease state.

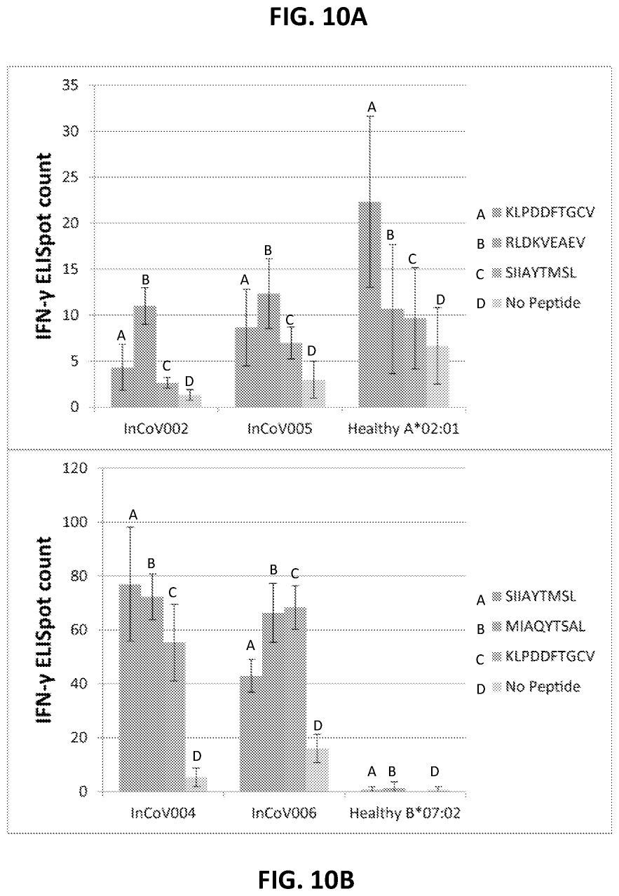

Although antigen-specific T cells were detected by NP-NACS, the degree to which these cells can be induced by those epitopes to produce an actual immune response remained in question. Five peptides comprising epitopes detected from either the A*02:01 or B*07:02 assay were synthesized and used in an ELISpot assay to stimulate HLA-matched PBMCs. In the A*02:01 assay (FIG. 10A), IFN-γ secretion was upregulated upon exposure to the peptides in both disease and healthy PBMCs. However, there was variation in the degree of IFN-γ upregulation per peptide. RLDKVEAEV (SEQ ID NO: 113) induced the strongest response in the INCOV PBMCs, whereas for healthy PBMCs, KLPDDFTGCV (SEQ ID NO: 114) elicited the strongest response, and to a greater extent when compared to other peptide responses seen in the INCOV samples. The fact that the KLPDDFTGCV (SEQ ID NO: 114) SCT captured the highest frequency of cells in NP-NACS but gave a significantly reduced IFN-g response in INCOV samples, while the healthy donor produced opposite results, indicates that this peptide perhaps is immunogenic but might cause T cell exhaustion in a disease state. A similar assay was performed for B*07:02 PBMCs using another set of peptides (FIG. 10B). Here, the healthy B*07:02 donor PBMCs had no response to stimulation by any peptide, while the INCOV PBMCs secreted IFN-γ only with peptide stimulation. However, it was not expected that KLPDDFTGCV (SEQ ID NO: 114) would induce IFN-γ secretion for these PBMCs, as it was a predicted binder only to A*02:01 HLA alleles. A deeper HLA analysis of the INCOV-004 sample revealed that this participant also was positive for A*02:01, so activation by this peptide was expected. INCOV-006, however, did not possess the A*02:01 haplotype. It may be that the KLPDDFTGCV (SEQ ID NO: 114) peptide can be presented by this participant's other HLA alleles.

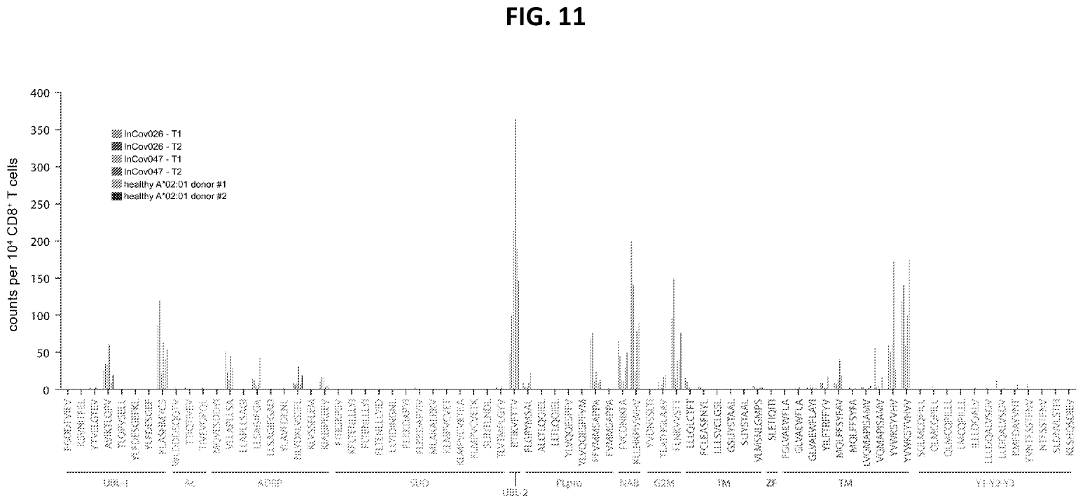

As reported in other virus studies, non-structural proteins tend to be preferential for CD8+ T cell activation. This finding, if applicable to the context of SARS-COV-2, would be highly informative towards targeted vaccine developments. One such domain of interest, Nsp3, encodes a papain-like protease (PLpro), which has been identified in other coronavirus strains to play a significant role in the early stages of the infection cycle, processing other non-structural elements that are responsible for infection and assembly of structural virus elements. As such, Nsp3 is expressed much earlier than structural elements such as the spike protein. Therefore, Nsp3 epitopes might be also be surveyed by the immune system earlier than epitopes derived from structural proteins. 191 Nsp3 peptide-encoded HLA-A*02:01 SCT plasmids were produced, approximately 100 of them expressed to a sufficient degree for biotinylation and tetramerization, and the top 75 expressed SCTs were utilized in NP-NACS to identify antigen-specific T cells in two COVID participants and two healthy controls (FIG. 11). Again, both healthy and COVID PBMCs showed reactivity to the same epitopes. However, the relative counts for PLpro epitopes were much higher than for spike epitopes. Surprisingly, for some epitopes, healthy PBMCs gave just as high of a response. This finding may imply prior exposure to coronavirus strains harboring similar epitopes.

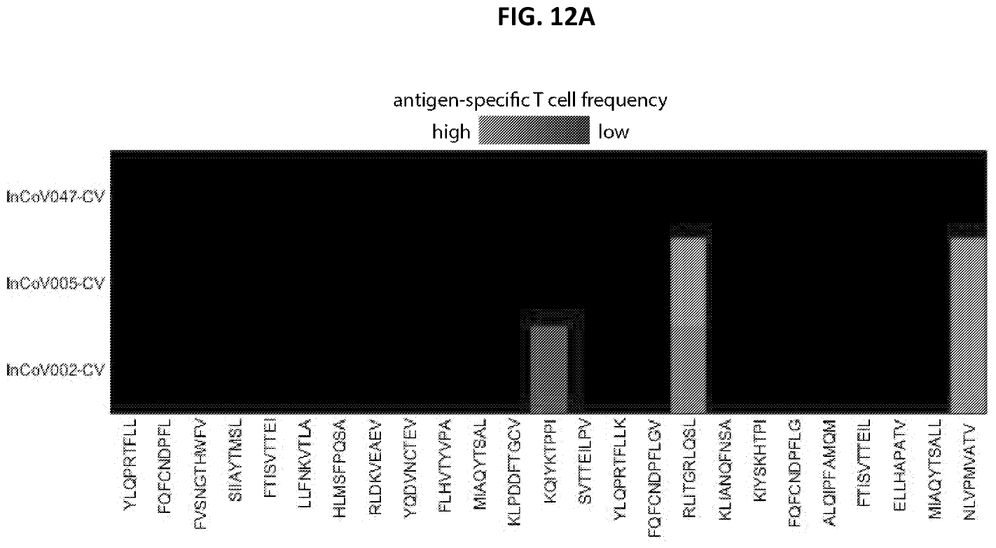

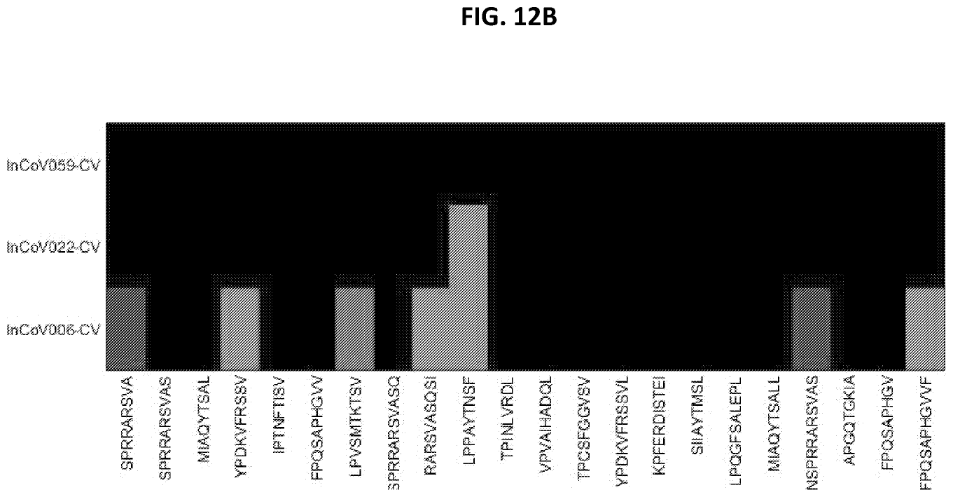

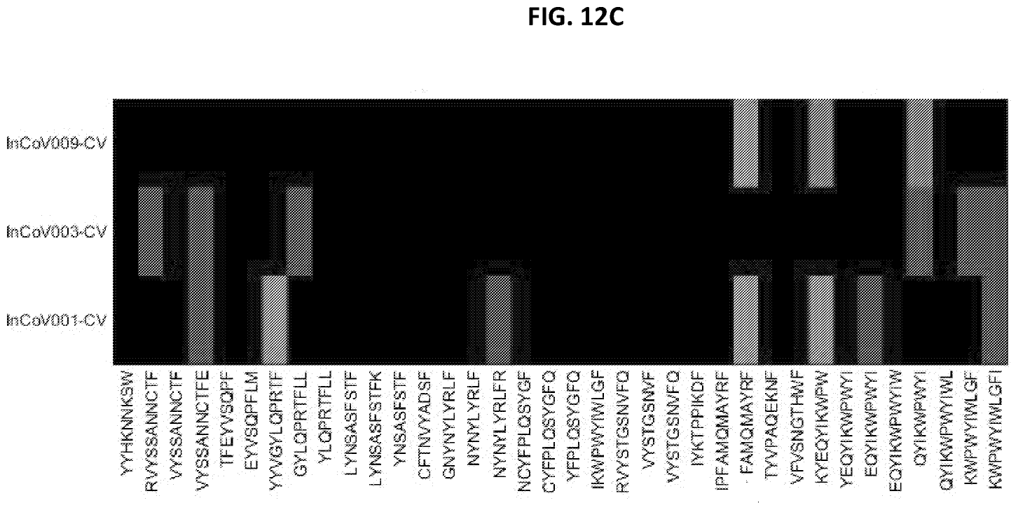

SCTs enable high-throughput discovery of SARS-COV-2-specific TCR sequences: While the NP-NACS platform allowed rapid identification of immunogenic antigens from primary CD8⁺ T cells, TCR sequences were needed for additional functional validation. Without the additional avidity conferred by the NP-NACS nanoparticle scaffold, tetramer/dextramer binding assays are expected to have some inherent degree of non-specific binding. This would render identification of antigen specificity difficult when working with primary CD8⁺ T cells due to their low frequency and generally lower cell quality. Therefore, cells were first sorted for primary CD8⁺ T cells using SCT tetramer pools for each patient (tetramer pools consisted of all SARS-COV-2 SCTs synthesized matching the participant's HLA haplotype). Each of the sorted populations were then expanded for approximately two weeks to improve quantity and viability. The cells were subsequently sorted by individual SCT tetramers within their respective libraries, such that associate each sorted population of TCR clonotypes could be associated with targeted antigen. NGS bulk sequencing of the samples revealed antigen-specific populations against a subset of spike and PLpro antigens across most patients (FIG. 12). Of the 21 unique peptides which had detectable T cell populations, eight of them were found across multiple patients. Two of the patient samples had no cells captured by bulk sequencing after the expansion process, perhaps due to poor viability or biased expansion of non-specific T cells after low counts of tetramer-binding cells were collected during the pooled tetramer sorting step.

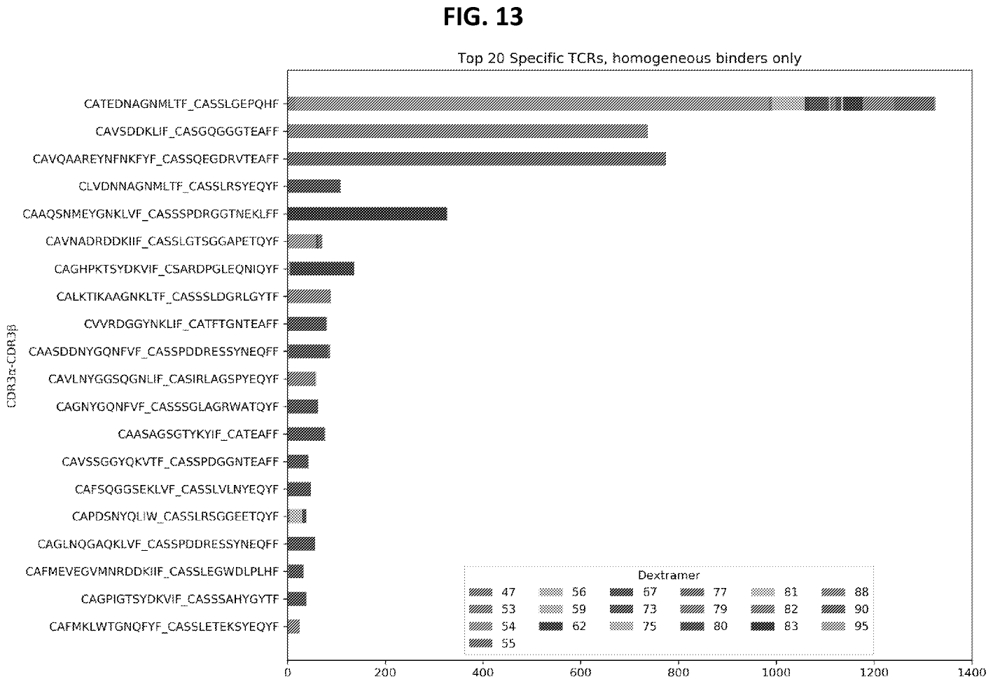

To complement the bulk sequencing approach, single-cell sequencing on the expanded T cell populations was carried out to identify any prevalent clonotypes that may have been missed. The expanded cells were stained with a DNA hashtag to encode patient identity. Then, they were stained with a designated set of SCT dextramers, each containing an antigen-encoded DNA barcode. After excess dextramers were washed, the stained T cells from multiple patients were combined together and submitted for 10× single-cell sequencing. To assess the quality of SCT capture, dextramer binding frequency and heterogeneity was quantified. The 10× data was first sorted to identify the top 20 clonotypes which had the highest frequency of homogeneous dextramer binding (signal only from one unique dextramer barcode per cell), encompassing a frequency range of 24 to 959 antigen-specific cells detected against the dominant dextramer per clonotype (FIG. 13 and Table 5). The dextramer IDs of these 20 clonotypes were traced back to their associated SCT identities to reveal specificity to six unique epitopes across A*02:01 and B*07:02. Five of the six epitopes were derived from spike protein, and one from PLpro. For each clonotype, cells with heterogeneously bound dextramers (non-specific) displayed a dominant dextramer signal derived from the same SCT as that of the homogeneously bound cells (not shown), but this signal comprised a significantly smaller fraction of the total dextramer signal. This indicates that the expansion step, followed by dextramer signal filtering, allows for successful reduction of background noise caused by non-specific dextramer binding. A comparison of the captured TCR data from single-cell against bulk sequencing revealed an overlap of six TCR clonotypes. The identified SCT specificity from five of these six clonotypes, whether in tetramer format (bulk sequencing) or dextramer format (10× single cell), were in agreement.

TABLE 5
Dextramers used per patient sample.

| Patient | Status | HLA | Antigen | Dextramer |
|---|---|---|---|---|
| InCoV003-CV | T3 | A*24:02 | KWPWYIWLGF (SEQ | 47 |
| | | | ID NO: 115) | |
| InCoV047-CV | T3 | A*02:01 | FCLEASFNYL (SEQ | 53 |
| | | | ID NO: 116) | |
| InCoV047-CV | T3 | A*02:01 | MLAKALRKV (SEQ | 54 |
| | | | ID NO: 117) | |
| InCoV047-CV | T3 | A*02:01 | YLQPRTFLLK (SEQ | 55 |
| | | | ID NO: 118) | |
| InCoV047-CV | T3 | A*02:01 | KQIYKTPPI (SEQ ID | 56 |
| | | | NO: 119) | |
| InCoV005-CV | T3 | A*02:01 | MLAKALRKV (SEQ | 59 |
| | | | ID NO: 120) | |
| InCoV005-CV | T3 | A*02:01 | RLITGRLQSL (SEQ | 62 |
| | | | ID NO: 121) | |
| InCoV002-CV | T3 | A*02:01 | MLAKALRKV (SEQ | 67 |
| | | | ID NO: 122) | |
| InCoV002-CV | T3 | A*02:01 | RLITGRLQSL (SEQ | 73 |
| | | | ID NO: 123) | |
| InCoV002-CV | T3 | A*02:01 | KQIYKTPPI (SEQ ID | 75 |
| | | | NO: 124) | |
| InCoV006-CV | T3 | B*07:02 | FPQSAPHGVVF (SEQ | 77 |
| | | | ID NO: 125) | |
| InCoV006-CV | T3 | B*07:02 | LPPAYTNSF (SEQ ID | 79 |
| | | | NO: 126) | |
| InCoV006-CV | T3 | B*07:02 | RARSVASQSI (SEQ | 80 |
| | | | ID NO: 127) | |
| InCoV006-CV | T3 | B*07:02 | YPDKVFRSSV (SEQ | 81 |
| | | | ID NO: 128) | |
| InCoV006-CV | T3 | B*07:02 | SPRRARSVA (SEQ ID | 82 |
| | | | NO: 129) | |
| GB17457 | Healthy | A*02:01 | RLITGRLQSL (SEQ | 83 |
| | | | ID NO: 130) | |
| GB17457 | Healthy | A*02:01 | LLFNKVTLA (SEQ ID | 88 |
| | | | NO: 131) | |
| GB18622 | Healthy | A*02:01 | RLITGRLQSL (SEQ | 90 |
| | | | ID NO: 132) | |
| GB18622 | Healthy | A*02:01 | LLENKVTLA (SEQ ID | 95 |
| | | | NO: 133) | |

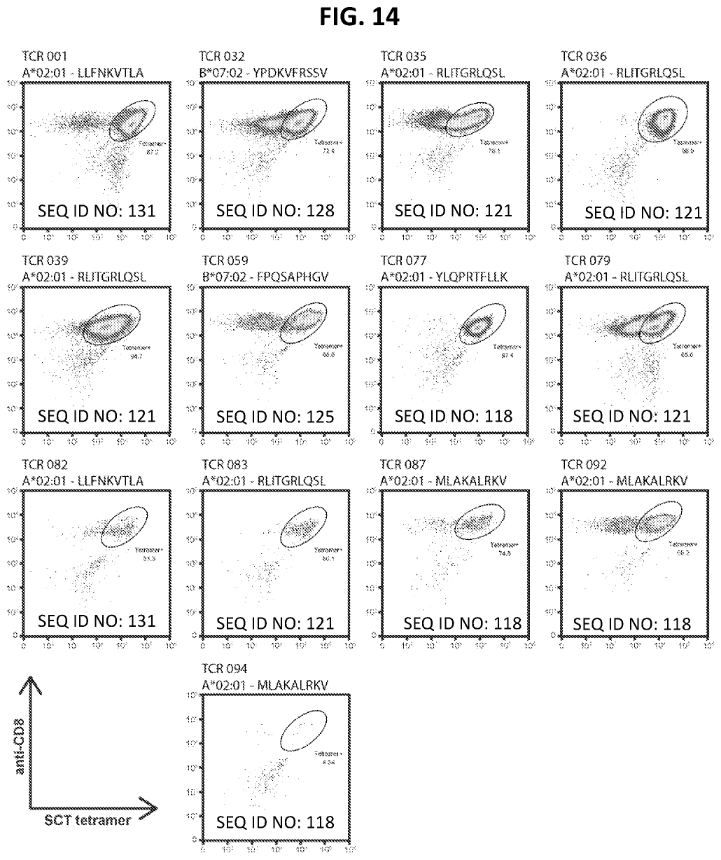

Identified TCRs are functionally responsive against SARS-COV-2 peptides: In order to functionally validate the TCRs, sequencing results from bulk and 10× single-cell methods were sorted by prevalence, and 86 unique SARS-COV-2-specific TCRs were selected for cloning into primary CD8⁺ T cells by CRISPR/Cas9 transduction. In order to thoroughly scan the most prevalent clonotypes for peptide specificity, several of the selected TCR clonotypes consist of different combinations of a/b pairs for cells in which dual TCR receptors were detected (e.g., TCR 087 & 092 share the same TCR β chain). The transduced T cells were sorted with SCT tetramers of corresponding antigen-specificity, and expanded for at least two weeks to generate cell lines. Of the 86 TCR sequences, at least 13 could specifically bind to SCT tetramers after expansion (FIG. 14). The lack of strong tetramer binding by the other T cell lines could be explained by the following causes: 1) non-productive TCR pairs derived from cells with dual TCRs; 2) collection of background cells from initial sorting of T cells from PBMCs via 10× or bulk method; 3) biased expansion of non-productive T cells. A larger proportion of 10×-derived TCR sequences were productive versus bulk-derived TCR sequences, due to enhanced precision of the single-cell sequencing approach.

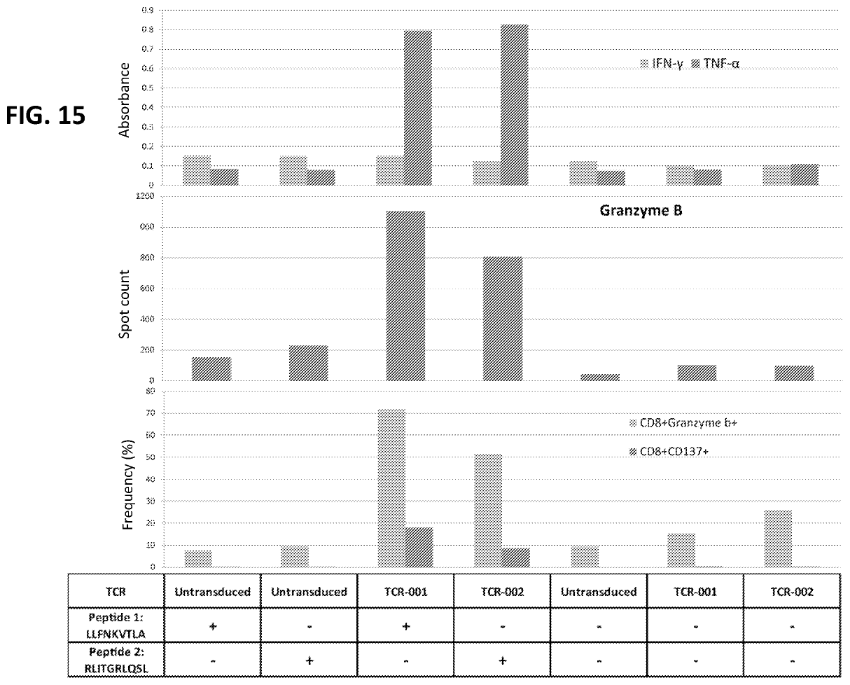

Initial functional validation of TCRs 001 and 002, which were obtained from healthy donors, demonstrated that peptide stimulation could induce CD137 expression (FIG. 15). This indicates that the TCRs identified are indeed capable of binding to biological pMHCs and inducing downstream activation signals. Furthermore, ELISA, ELISpot, and flow cytometry assays demonstrated that peptide-stimulated T cells could be induced to release cytokines (specifically, TNF-α was observed but not IFN-γ) and proteases (granzyme B), characteristic of a cytotoxic response from CD8⁺ T cells upon activation (FIG. 15).

### Example 6

### Impact of CD8-Inhibiting Mutations on SCT Function

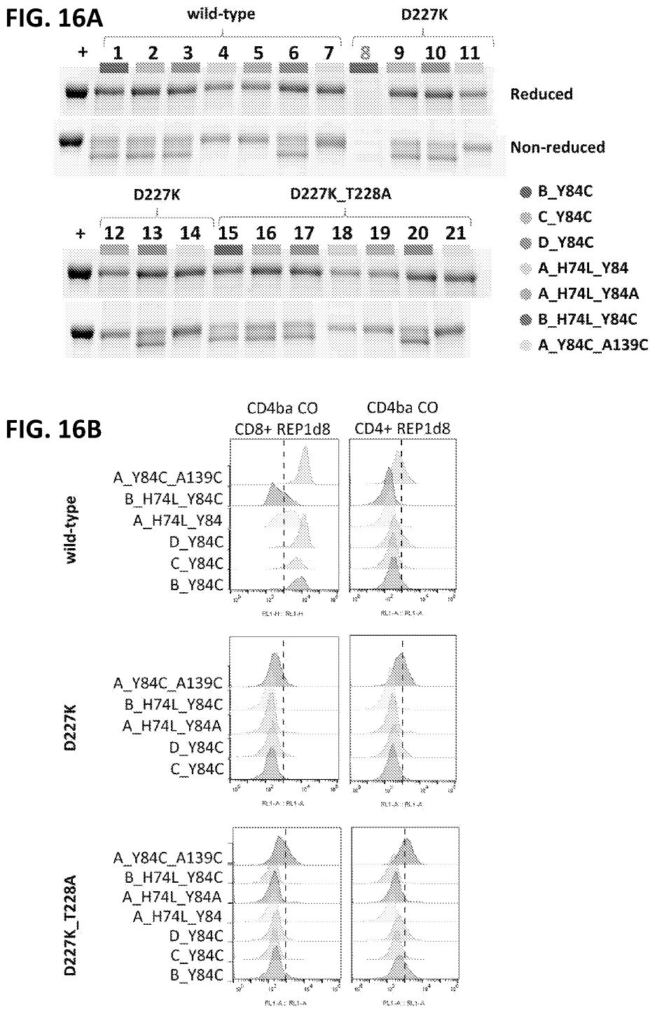

Two mutations that have previously been reported to block CD8 interaction with pMHCs (D227K and D227K+T2238A) were implemented into the SCT platform to generate a small library of A*02:01 SCT variants loaded with the WT1 peptide (RMFPNAPYL; SEQ ID NO: 1). The WT1 SCTs were capable of expression only for certain template variations (FIG. 2B and FIG. 16A). The plasmid templates which successfully led to expression were subsequently mutated to introduce either D227K or D227K+T228A together across all templates. Standard transfection of this library, encompassing seven core templates across three CD8-interaction variants (wild-type (no HLA mutation), D227K, or D227K+T228A), was performed over four days, and SDS-PAGE was conducted to characterize the yield (FIG. 16A). A WT1-specific A*02:01-restricted TCR (CD4ba) was transduced either into a CD8⁺ or a CD4⁺ cell line, to assess the impact of various HLA mutations on their capacity to interact with the CD8 co-receptor.

Transfection of these SCTs led to template-dependent yields across each HLA mutation type. Templates D6 and D7, which do not contain a cysteine-modified L1 linker and which also do not have the pocket-stabilizing dithiol mutation seen in D9, gave consistently lower yields relative to other templates. A double-banding pattern of SCTs was observed in a non-reduced SDS-PAGE environment. Because this pattern was only observed in templates which implemented a cysteine linker, this is believed to be the cause of double-banding, but it is not expected to have any impact on function (see top left plot of FIG. 16B).

The tetramer binding assays against TCR-transduced cell lines showed distinctive binding patterns for each HLA variant. The wild-type SCTs, when used to stain TCR-transduced CD8+ T cells, displayed variable degrees of successful binding to the cognate TCR (FIG. 16B and FIG. 4). Among this wild-type subset, D3 and D9 templates showed remarkably high binding efficiency, capturing at least 90% of all cells. When either D227K or D227K_T228A mutations were introduced, essentially complete abolishment of TCR binding across all SCT variants occurred (FIG. 16B, middle-left and bottom-left plots), except for D227K_T228A D9 variant, which still showed some degree of binding capability.

TCR-transduced CD4+ T cells were also tested to see if the absence of CD8 on these T cells might still result in binding by any SCT variant. As seen in the top-row plots of FIG. 16B, the wild-type SCTs showed a drastic reduction in binding against CD4+ T cells compared to binding against CD8+ T cells, indicating that most of these SCT variants relied on the CD8 co-receptor to facilitate pMHC-TCR affinity. For all SCT variants which contained a CD8-inhibiting mutation, binding efficiencies against CD8+ or CD4+ T cells were virtually unchanged.

Across all cell lines and HLA mutations, the D9 SCT template appeared to be the best binder in terms of signal retention beyond the 10³ MFI threshold. Indeed, as seen across all cases where CD8 interaction is removed (either with introduction of CD8-inhibitory mutation or substitution of CD8 with CD4), the D9 tetramer was capable of still generating some signal beyond noise. D9 SCTs for other peptides and other HLAs do not non-specifically bind (not shown). Thus, these results are interpreted to indicate that the D9 template may be improved compared to other designs in terms of epitope presentation for enhanced affinity against TCR.

Another HLA mutation, A245V, has been previously demonstrated to reduce CD8 interaction with pMHCs during TCR activation. This mutation was implemented for a private neoantigen-encoded library of SCTs, showing its capacity to significantly reduce background noise from binding of non-specific T cells. A*03:01 SCTs (D3 template) with the A245V mutation was generated for A*03:01-restricted peptides of a melanoma patient. The expression results of this library (data not shown) matched in terms of expressed protein band intensity per peptide-encoded SCT against its wild-type (no A245V mutation) variant, indicating that the mutation had no significant impact on protein expression capabilities of transfected cells. Subsequently, the biotinylated, purified SCTs were tetramerized for use against PBMC samples from the melanoma patient to detect antigen-specific T cells. The tetramers were utilized in groups of three to assess for three antigen specificities per flow experiment, where one antigen specificity was tetramerized with streptavidin-PE while the other two specificities were tetramerized with streptavidin-APC. In this manner, detection of double-positive fluorescence signal would indicate non-specific cross-binding of SCT tetramers. Cells which exhibit significant PE signal but not APC would be truly specific T cells.

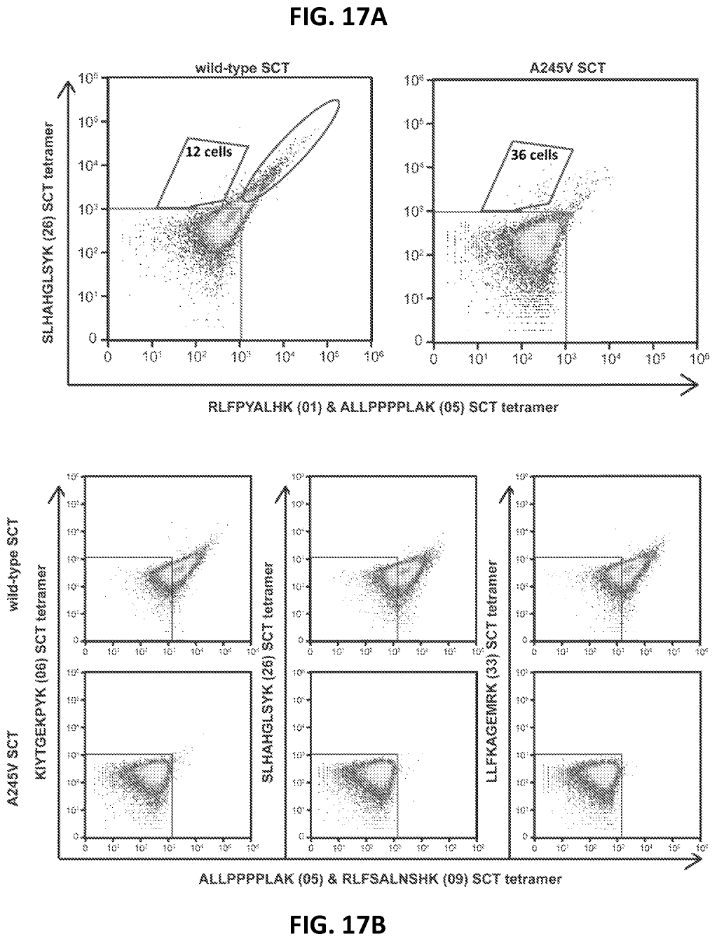

When this experiment was performed for a set of three SCTs without using the A245V mutation (FIG. 17A), significant cross-binding was observed, strongly skewing the tetramer-bound populations into a diagonal on the flow plot. However, when the A245V mutation was implemented for SCTs of the same antigen specificities, this cross-bound population was essentially removed. Furthermore, the counts of SLHAHGLSYK (SEQ ID NO: 134)-specific T cells based on PE-specific signal (polygonally bound region) increased. This suggests that without the A245V mutation, non-specifically bound cells overwhelm the tetramer-positive population, in essence masking the true positive reads from being properly detected. Once the A245V mutation was inserted to inhibit CD8 interaction, some of the truly PE-specific population (found in the oval-bound region of left in FIG. 17A) will only bind to the peptide-associated PE tetramer, thus increasing PE-specific binding counts.

This experimental setup was repeated three additional times (FIG. 17B), with each case having a unique arrangement of one SCT tetramerized with streptavidin-PE and two SCTs tetramerized with streptavidin-APC. In all cases, when comparing binding results of wild-type SCTs versus A245V SCTs, there were two major observations. First, the overall signal intensity decreased such that most cells gave below 10³ MFI (the cutoff threshold for establishing specific binding). Second, for A245V SCT tetramer staining, cells which generated a signal beyond 10³ MFI tend to only do so in one axis, indicating that may only have specificity to one of the three SCT tetramers assessed. This is in strong contrast to what is observed with the wild-type SCTs, where again, similar to FIG. 17A, there was clearly a strong inclination for non-specific binding events to occur to generate a skewed diagonal.

Similar to the SCT library production design for the A*03:01 neoantigens, an A*02:01 SCT library (D8 design) containing the A245V mutation was generated to encode various A*02:01 viral and bacterial peptides. Four of these elements were selected to be utilized in tetramer binding assays against PBMCs obtained from a healthy A*02:01 donor sample, where for each assay, one of three viral peptide SCT elements (tetramerized with streptavidin-PE) was mixed with the bacterial SCT element (tetramerized with streptavidin-APC) prior to staining. The viral SCTs encode peptides derived from EBV, CMV, and influenza viruses which have been reported in the literature to have cognate TCRs in virtually all A*02:01 individuals, whereas the bacterial SCT encodes a peptide from M. tuberculosis, for which not much reactivity was expected, given the low prevalence of this disease. Therefore, the former elements serve essentially as positive controls in the staining assay, while the latter element serves as a negative control.

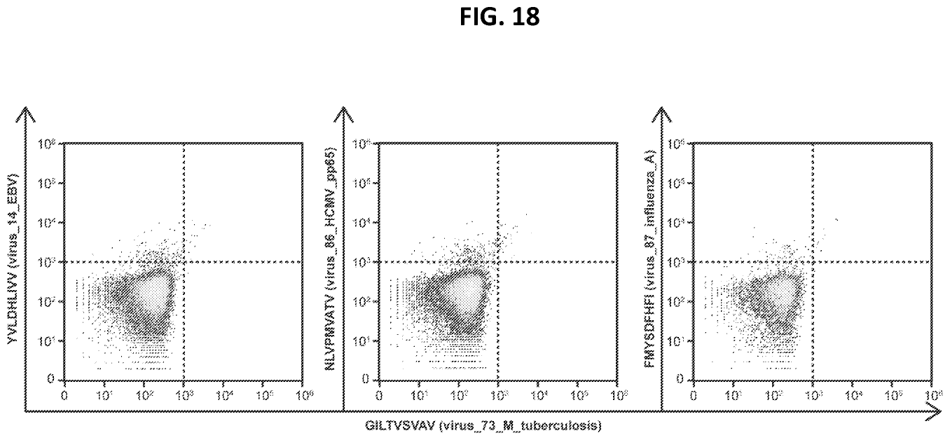

As seen in FIG. 18, the flow cytometry results for these A245V SCTs displayed a remarkably similar profile to that of the A*03:01 A245V SCTs (FIG. 17B), where most of the staining signal was contained within the lower left quadrant and no diagonal skew was present. This is highly suggestive of a strong reduction in non-specific binding compared to wild-type SCTs. Furthermore, for cells which do generate a positive signal in this experiment, in all three assays, this was only observed for tetramer-PE, indicating specific binding only by tetramers designed to present a common viral epitope. The lack of any binding by the M. tuberculosis antigen SCT tetramers is in alignment with the expectation for negative control results.

### Example 7

### Impact of Peptide Length on SCT Expression

During the initial analysis of SCT expression across various templates (FIG. 2), the 12mer YML peptide was surprisingly found to be capable of expression. The peptide sequence of the HPV E7 protein (YMLDLQPETTDLYC; SEQ ID NO: 5) was adapted into lengths of 8 to 14 amino acids. Primers encoding these peptides were utilized in inverse PCR reactions to insert these codons into the peptide region of A*02:01 SCT templates (eight designs total). The plasmids were transfected into Expi293 cells, incubated for four days, and the SCT expression was measured by SDS-PAGE analysis. The SCTs were further assessed for thermal stability by performing thermal shift assays.

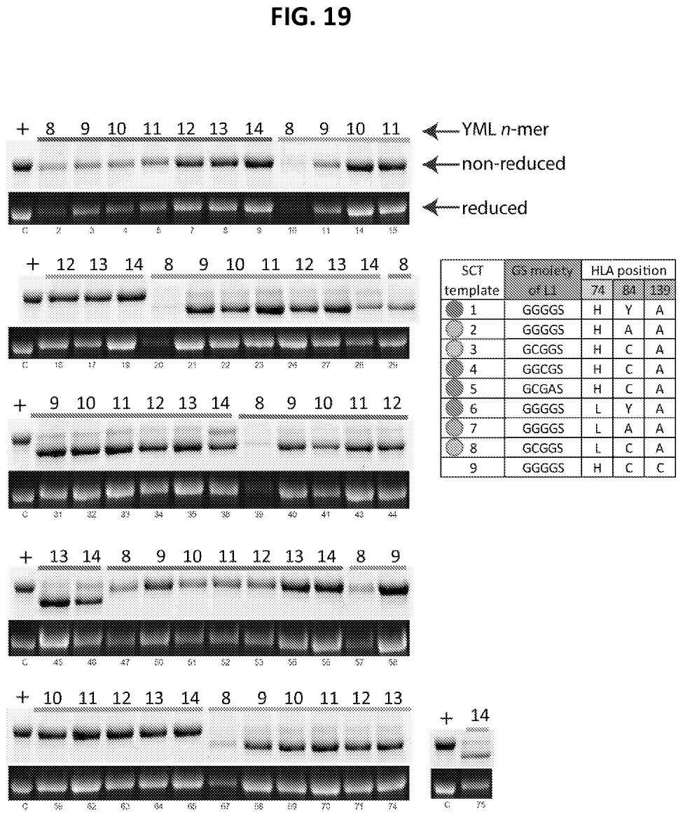

All SCTs containing the YML 8mer produced the weakest expression in general (FIG. 19). The highest expression yields across all design templates for the 8mer peptide were for those which used template designs without a cysteine linker (D1, D2, D6, D7). One hypothesis is that the cysteine linkers force the 8mer into a configuration within the HLA's binding pocket that is not amenable to stabilization and expression. Amongst the 8mer SCTs which had high yield, the D1 variant produced higher expression than D2, indicating that the Y84A mutation might be slightly worse at stabilization. The expression difference between these two templates is moderately reduced when the H74L mutation is added (D6 vs D7), where the yields appear comparable.

SCTs with 9mer to 13mer peptides showed consistent expression levels across all templates. 9mer, 10mer, 11mer SCTs had relatively worse expression than other peptides for D1, while the 9mer was relatively worse for D2. Similar to the 8mer, the 14mer seemed to experience significantly reduced expression when using a cysteine linker template. For D3, D4, D5, D8, the 14mer showed significantly lower expression compared to 9-13mers of the same templates. However, for non-cysteine linker templates (D1, D2, D6, D7), the 14mer ranked among the high-expressing SCTs compared to peptides of the same template. The 14mer may be constrained by the presence of a cysteine linker. By forcing all amino acids upstream of the cysteine link to fit in the binding groove ahead of the C-terminal pocket enclosure, there is a high likelihood that the steric hindrance introduced will not enable the epitope to remain as stably bound to the groove. This issue becomes more apparent as the peptide length increases, explaining the expression differences of the 14mer versus other lengths.

All templates with cysteine linker displayed a double-banding pattern in non-reduced SDS-PAGE. This is a template-dependent phenomenon, similar to what was previously observed when expressing WT1 SCTs.

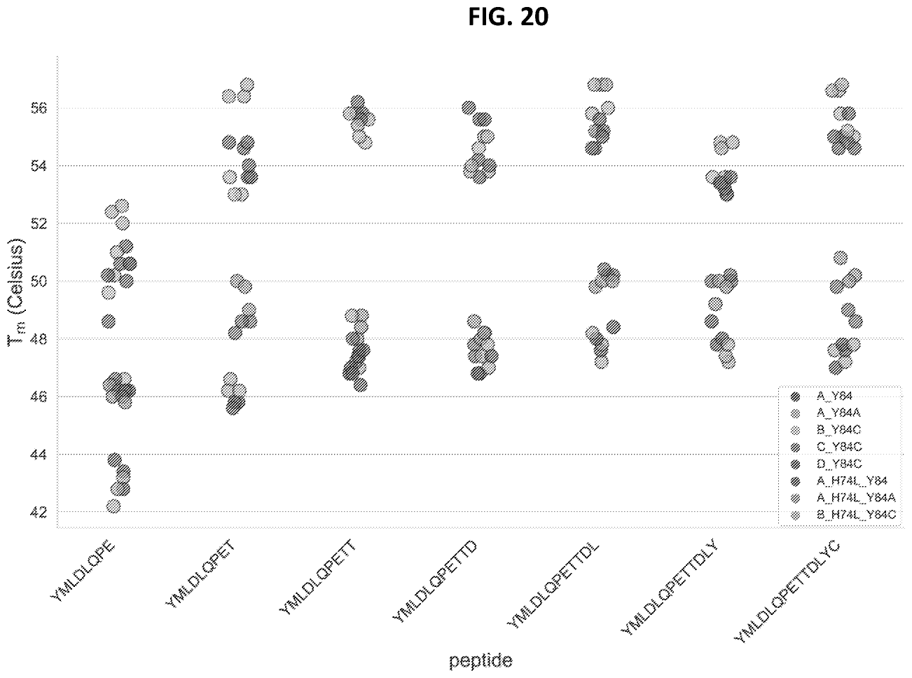

To further assess the stability of these SCTs, melting temperatures of the proteins were performed. As seen in FIG. 20, Tₘ values across the peptide series showed dependencies on SCT template and peptide length. Across all the peptides, the most stable constructs consisted of templates which made use of a cysteine linker template. Templates without a cysteine mutation experienced a drastic reduction in melting temperature, dropping by approximately 6° C.

The H74L mutation was also another significant factor which increased protein stability. When comparing templates which are identical except for the presence of this mutation (D1 vs. D6, D2 vs. D7, D3 vs. D8), the template with the H74L mutation was typically more stable. When Tₘ values were examined on the basis of peptide length, there was a clear drop in stability for 8-mer SCTs. Beyond this length, all SCTs experienced substantial improvement in stability, but there was no clear Tₘ difference per template for 9mer to 14mer, with the exception of the 9mers, for which D1 and D2 appear to afford slightly less stability than what would be expected of their counterparts for 10mers or longer. The most stable template across all templates was consistently the D8 template. The H74L mutation most likely explains the improved stabilization, given that D3 SCTs (which do not contain the H74L mutation) were always less stable than D8.

### Example 8

### Adoptive Transfer Cell Therapy

This example describes methods that can be used to produce a population of T cells expressing an antigen-specific T cell receptor and administering the cells to a subject. While particular methods are provided, one of skill in the art will recognize that methods that deviate from these specific methods can also be used, including addition or omission of one or more steps.

An exemplary method for identifying antigen-specific T cell receptors from a subject, such as a subject with a tumor and administering a population of T cells expressing the TCRs to the subject is schematically illustrated in FIG. 21. Healthy (non-tumor) tissue and tumor tissue is extracted and analyzed by sequencing of the transcriptome to identify neoantigens and also the HLA haplotype of the subject. Peptide-MHC binding affinity predictions are performed to identity the best peptide candidates of the neoantigen for pMHC generation. Stable pMHCs are then produced and tetramerized as described herein. These are used to capture antigen-specific T cells. TCRs from the captured T cells are sequenced and synthesized in plasmid expression constructs. These are transformed into healthy T cells and administered to the subject by adoptive cell therapy protocols. In some examples, the antigen-specific T cells, the transformed T cells, or both are from the subject being treated, but in other examples, one or both could be from another subject.

### EMBODIMENTS OF THE DISCLOSURE

Embodiment 1 includes a nucleic acid fragment pair comprising a first nucleic acid fragment and second nucleic acid fragment that, when assembled, encode a major histocompatibility complex (MHC) Class I single chain trimer (SCT) protein, the SCT comprising as operably linked subunits a peptide, a β2 microglobulin (β2m) protein, and a human leukocyte antigen (HLA) protein, and wherein the first nucleic acid fragment and the second nucleic acid fragment each comprise a portion of an assembly site in the β2 microglobulin protein.

Embodiment 2 includes the nucleic acid fragment pair of embodiment 1, wherein the assembly site is a Gibson assembly site.

Embodiment 3 includes the nucleic acid fragment pair of embodiment 1 or 2, wherein the MHC Class I SCT protein encoded by the assembled nucleic acid fragment pair comprises protein subunits encoded in the following order: secretion signal, peptide, peptide-β2m linker (L1), β2m, β2m-HLA linker (L2), HLA, and optionally, one or more purification tags, and wherein the assembly site is positioned within an invariant region of β2m.

Embodiment 4 includes the nucleic acid fragment pair of embodiment 3, wherein the secretion signal is selected from an HLA secretion signal, an interferon-α2 secretion signal, and an interferon-γ secretion signal.

Embodiment 5 includes the nucleic acid fragment pair of embodiment 3 or 4, wherein the MHC Class I SCT protein comprises one or more purification tags and the one or more purification tags are selected from a peptide that can be biotinylated and a polyhistidine peptide.

Embodiment 6 includes the nucleic acid fragment pair of any one of embodiments 1 to 5, wherein the second nucleic acid fragment encodes a HLA protein comprising one or more amino acid substitutions selected from the group consisting of H74L, D74L, Y84C, Y84A, A139C, D227K, T228A, and A245V, wherein the amino acid position corresponds to SEQ ID NO: 3.

Embodiment 7 includes the nucleic acid fragment pair of any one of embodiments 1 to 6, wherein the peptide is an antigen peptide, a self peptide, or a placeholder peptide.

Embodiment 8 includes the nucleic acid fragment pair of embodiment 7, wherein the antigen peptide is selected from a tumor-associated peptide, a neoantigen peptide, an autoimmune peptide, a fungal peptide, a bacterial peptide, and a viral peptide.

Embodiment 9 includes the nucleic acid fragment pair of any one of embodiments 1 to 8, wherein the nucleic acid fragment pair is codon-optimized for mammalian expression.

Embodiment 10 includes a nucleic acid molecule comprising the assembled nucleic acid fragment pair of any one of embodiments 1 to 9, wherein the assembled nucleic acid fragment pair comprises the first nucleic acid fragment operably linked to the second nucleic acid fragment.

Embodiment 11 includes a vector comprising the nucleic acid molecule of embodiment 10.

Embodiment 12 includes the vector of embodiment 11, wherein the vector is a mammalian expression vector.

Embodiment 11 includes the vector of embodiment 12, wherein the mammalian expression vector is plasmid pcDNA3.1.

Embodiment 14 includes a human cell line transformed with the vector of any one of embodiments 11 to 13.

Embodiment 15 includes the human cell line of embodiment 14, wherein the cell line is an HEK293 cell line.

Embodiment 16 includes the human cell line of embodiment 15, wherein the cell line is Expi293F™ cell line.

Embodiment 17 includes a library comprising a plurality of the nucleic acid fragment pairs of any one of embodiments 1 to 9.

Embodiment 18 includes a library comprising a plurality of the assembled nucleic acid fragment pairs of embodiment 17.

Embodiment 19 includes a human-glycosylated MHC Class I single chain trimer (SCT) protein.

Embodiment 20 includes the human-glycosylated MHC Class I SCT protein of embodiment 19, wherein the SCT protein is soluble.

Embodiment 21 includes the soluble human-glycosylated MHC Class I SCT protein of embodiment 20, comprising an antigen peptide, a self peptide, or a placeholder peptide.

Embodiment 22 includes the soluble human-glycosylated MHC Class I SCT protein of embodiment 21, wherein the antigen peptide is selected from a tumor-associated peptide, a neoantigen peptide, an autoimmune peptide, a fungal peptide, a bacterial peptide, and a viral peptide.

Embodiment 23 includes the soluble human-glycosylated MHC Class I SCT protein of any one of embodiments 20 to 22, comprising a peptide, a peptide-β2 microglobulin (β2m) protein linker (L1), a β2m protein, a β2m-HLA linker (L2), and an HLA protein, in N-terminal to C-terminal order.

Embodiment 24 includes the human-glycosylated MHC Class I SCT protein of embodiment 23, wherein the HLA protein comprises one or more amino acid substitutions selected from the group consisting of H74L, D74L, Y84C, Y84A, A139C, D227K, T228A, and A245V, wherein the amino acid position corresponds to SEQ ID NO: 3.

Embodiment 25 includes the soluble human-glycosylated MHC Class I SCT protein of embodiment 23 or 24, further comprising one or more purification tags.

Embodiment 26 includes the soluble human-glycosylated MHC Class I SCT protein of embodiment 25, wherein the one or more purification tags are selected from a peptide that can be biotinylated and a polyhistidine peptide.

Embodiment 27 includes the soluble human-glycosylated MHC Class I SCT protein of any one of embodiments 20 to 26, wherein the SCT protein is assembled as a stable multimer.

Embodiment 28 includes the soluble human-glycosylated MHC Class I SCT protein of embodiment 27, wherein the stable multimer is a tetramer.

Embodiment 29 includes the soluble human-glycosylated MHC Class I SCT protein of embodiment 27 or 28, wherein the stable multimer is attached to a polymer or a nanoparticle scaffold.

Embodiment 30 includes a library comprising a plurality of soluble human-glycosylated MHC Class I SCT proteins of any one of embodiments 20 to 26.

Embodiment 31 includes a library comprising a plurality of stable multimers of any one of embodiments 27 to 29.

Embodiment 32 includes a method of identifying an antigen-specific CD8⁺ T cell, comprising:
contacting a T cell population with one or more of the stable multimers of a soluble human glycosylated MHC Class I SCT protein of embodiments 27 to 29; and
identifying a CD8⁺ T cell reactive thereto.

Embodiment 33 includes the method of embodiment 32, further comprising:
sequencing the T cell receptor (TCR) of the identified antigen-specific CD8⁺ T cell; and
producing a population of T cells expressing the antigen-specific TCR.

Embodiment 34 includes the method of embodiment 33, further comprising administering the population of T cells expressing the antigen-specific TCR to a subject in need thereof.

Embodiment 35 includes the method of embodiment 34, wherein the subject has cancer and the antigen-specific TCR is reactive to an antigen from a tumor sample obtained from the subject.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims

1. A nucleic acid fragment pair comprising a first nucleic acid fragment and second nucleic acid fragment that, when assembled, encode a major histocompatibility complex (MHC) Class I single chain trimer (SCT) protein, the SCT comprising as operably linked subunits a peptide, a β2 microglobulin (β2m) protein, and a human leukocyte antigen (HLA) protein, and wherein the first nucleic acid fragment and the second nucleic acid fragment each comprise a portion of an assembly site in the β2 microglobulin protein.

2. The nucleic acid fragment pair of claim 1, wherein the assembly site is a Gibson assembly site.

3. The nucleic acid fragment pair of claim 1, wherein the MHC Class I SCT protein encoded by the assembled nucleic acid fragment pair comprises protein subunits encoded in the following order: secretion signal, peptide, peptide-β2m linker (L1), β2m, β2m-HLA linker (L2), HLA, and optionally, one or more purification tags, and wherein the assembly site is positioned within an invariant region of β2m.

4. The nucleic acid fragment pair of claim 3, wherein the secretion signal is selected from an HLA secretion signal, an interferon-α2 secretion signal, and an interferon-γ secretion signal.

5. The nucleic acid fragment pair of claim 3, wherein the MHC Class I SCT protein comprises one or more purification tags and the one or more purification tags are selected from a peptide that can be biotinylated and a polyhistidine peptide.

6. The nucleic acid fragment pair of claim 1, wherein the second nucleic acid fragment encodes a HLA protein comprising one or more amino acid substitutions selected from the group consisting of H74L, D74L, Y84C, Y84A, A139C, D227K, T228A, and A245V, wherein the amino acid position corresponds to SEQ ID NO: 3.

7. The nucleic acid fragment pair of claim 1, wherein the peptide is an antigen peptide, a self peptide, or a placeholder peptide.

8. (canceled)

9. The nucleic acid fragment pair of claim 1, wherein the nucleic acid fragment pair is codon-optimized for mammalian expression.

10. A nucleic acid molecule comprising the assembled nucleic acid fragment pair of claim 1, wherein the assembled nucleic acid fragment pair comprises the first nucleic acid fragment operably linked to the second nucleic acid fragment.

11. A vector comprising the nucleic acid molecule of claim 10.

12-13. (canceled)

14. A human cell line transformed with the vector of claim 11.

15-16. (canceled)

17. A library comprising a plurality of the nucleic acid fragment pairs of claim 1.

18. A library comprising a plurality of the assembled nucleic acid fragment pairs of claim 17.

19. A human-glycosylated MHC Class I single chain trimer (SCT) protein.

20. The human-glycosylated MHC Class I SCT protein of claim 19, wherein the SCT protein is soluble.

21. The soluble human-glycosylated MHC Class I SCT protein of claim 20, comprising an antigen peptide, a self peptide, or a placeholder peptide.

22. (canceled)

23. The soluble human-glycosylated MHC Class I SCT protein of claim 20, comprising a peptide, a peptide-β2 microglobulin (β2m) protein linker (L1), a β2m protein, a β2m-HLA linker (L2), and an HLA protein, in N-terminal to C-terminal order.

24. The human-glycosylated MHC Class I SCT protein of claim 23, wherein the HLA protein comprises one or more amino acid substitutions selected from the group consisting of H74L, D74L, Y84C, Y84A, A139C, D227K, T228A, and A245V, wherein the amino acid position corresponds to SEQ ID NO: 3.

25. The soluble human-glycosylated MHC Class I SCT protein of claim 23, further comprising one or more purification tags.

26. (canceled)

27. The soluble human-glycosylated MHC Class I SCT protein of claim 20, wherein the SCT protein is assembled as a stable multimer.

28. (canceled)

29. The soluble human-glycosylated MHC Class I SCT protein of claim 27, wherein the stable multimer is attached to a polymer or a nanoparticle scaffold.

30. A library comprising a plurality of soluble human-glycosylated MHC Class I SCT proteins of claim 20.

31. A library comprising a plurality of stable multimers of claim 27.

32. A method of identifying an antigen-specific CD8⁺ T cell, comprising:
contacting a T cell population with one or more of the stable multimers of a soluble human glycosylated MHC Class I SCT protein of claim 27; and
identifying a CD8⁺ T cell reactive thereto.

33. The method of claim 32, further comprising:
sequencing the T cell receptor (TCR) of the identified antigen-specific CD8⁺ T cell; and
producing a population of T cells expressing the antigen-specific TCR.

34. The method of claim 33, further comprising administering the population of T cells expressing the antigen-specific TCR to a subject in need thereof.

35. (canceled)
